(19) United States
(12) Patent Application Publication
(10) Pub. No.: US 2025/0248970 A1
(43) Pub. Date: Aug. 7, 2025

(54) NOVEL SUBSTITUTED FLUORINATED N-PROPYL-PYRROLIDINE COMPOUNDS, PROCESSES FOR THEIR PREPARATION AND THERAPEUTIC USES THEREOF

(71) Applicant: SANOFI, Paris (FR)

(72) Inventors: Patrick BERNARDELLI, Paris (FR); Béatrice DE BRUIN, Paris (FR); Youssef EL-AHMAD, Paris (FR); Frédéric PETIT, Paris (FR); Franck SLOWINSKI, Paris (FR)

(21) Appl. No.: 18/856,297

(22) PCT Filed: Apr. 14, 2023
(86) PCT No.: PCT/EP2023/059772

§ 371 Date: Oct. 11, 2024

(30) Foreign Application Priority Data
Apr. 15, 2022 (EP) 22305562.5

(51) Int. Cl. A61K 31/4025; A61K 31/40; C07D 207/08; C07D 409/10

(52) U.S. Cl. CPC A61K 31/4025; A61K 31/40; C07D 207/08; C07D 409/10

(57) ABSTRACT

Disclosed herein are compounds of the formula (I), or pharmaceutically acceptable salts thereof, wherein R1 and R2 represent a hydrogen atom or a deuterium atom; R3 represents a hydrogen atom, a —COOH group or a —OH group; R3′ and R3″ represent a hydrogen atom, a methyl group, a methoxy group, a chlorine atom, a fluorine atom, or a cyano group; R4 and R5 represent a hydrogen atom, a halogen atom, a —NH2 group, a (C₁-C₃)alkyl group, a (C₁-C₃)alkoxy group, or a —OH group; or R4 and R5 together form an oxo group or R4 and R5 together form a ═NOCH3 group or a (C₃-C₅)cycloalkyl group; R7 represents a hydrogen atom, a methyl group, a —OH group or a fluorine atom; R6 represents a phenyl group, a fused phenyl group, a bicyclic group comprising 5 to 12 carbon atoms, a heteroaryl group comprising 2 to 9 carbon atoms and comprising from 1 to 3 heteroatoms, a cycloalkyl group comprising 3 to 7 carbon atoms, a (C₃-C₆)cycloalkyl(C1-C3)alkyl group, a 4 to 7 membered-heterocycloalkyl group, a (C₁-C₆)alkyl group or a phenyl(C₁-C₂)alkyl group; X represents —CH2-, —O— or —S—; Y represents —CH═, —N═ or —CR″═; R8 represents a (C₁-C₃)alkyl group, a halogen atom, a cyano group, or a (C₁-C₃)fluoroalkyl group; R9 represents a hydrogen atom or a fluorine atom; R10 and R10′ represent a hydrogen atom or a fluorine atom; R11 represents a hydrogen atom a (C₁-C₃)alkyl group or a cyclopropyl group; n is 0, 1 or 2, and m is 0 or 1. Further disclosed are process for preparing the same, pharmaceutical compositions comprising them as well as said compounds of formula (I) for use as an inhibitor and degrader of estrogen receptors, in particular in the treatment of ovulatory dysfunction, cancer, endometriosis, osteoporosis, benign prostatic hypertrophy or inflammation.

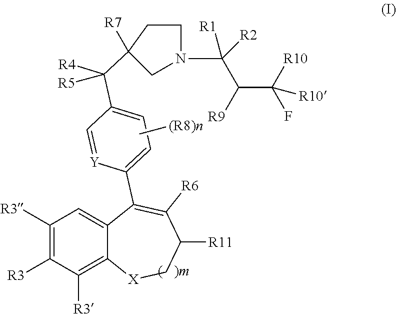

Disclosed herein are novel fluorinated N-propyl-pyrrolidine derivatives, the processes for their preparation, as well as the therapeutic uses thereof, in particular as anticancer agents via selective antagonism and degradation of estrogen receptors.

The Estrogen Receptors (ER) belong to the steroid/nuclear receptor superfamily involved in the regulation of eukaryotic gene expression, cellular proliferation and in target tissues. ERs are in two forms: the estrogen receptor alpha (ERα) and the estrogen receptor beta (ERβ) respectively encoded by the ESR1 and the ESR2 genes. ERα and ERβ are ligand-activated transcription factors which are activated by the hormone estrogen (the most potent estrogen produced in the body is 17β-estradiol). In the absence of hormone, ERs are largely located in the cytosol of the cell. When the hormone estrogen binds to ERs, ERs migrate from the cytosol to the nucleus of the cell, form dimers and then bind to specific genomic sequences called Estrogen Response Elements (ERE). The DNA/ER complex interacts with co-regulators to modulate the transcription of target genes.

ERα is mainly expressed in reproductive tissues such as uterus, ovary, breast, bone and white adipose tissue. Abnormal ERα signaling leads to development of a variety of diseases, such as cancers, metabolic and cardiovascular diseases, neurodegenerative diseases, inflammation diseases and osteoporosis.

ERα is expressed in not more than 10% of normal breast epithelium but approximately 50-80% of breast tumors. Such breast tumors with high level of ERα are classified as ERα-positive breast tumors. The etiological role of estrogen in breast cancer is well established and modulation of ERα signaling remains the mainstay of breast cancer treatment for the majority ERα-positive breast tumors. Currently, several strategies for inhibiting the estrogen axis in breast cancer exist, including: 1—blocking estrogen synthesis by aromatase inhibitors that are used to treat early and advanced ERα-positive breast cancer patients; 2—antagonizing estrogen ligand binding to ERα by tamoxifen which is used to treat ERα-positive breast cancer patients in both pre- and post-menopausal setting; 3-antagonizing and downregulating ERα levels by fulvestrant, which is used to treat breast cancer in patients that have progressed despite endocrine therapies such as tamoxifen or aromatase inhibitors.

Although these endocrine therapies have contributed enormously to reduction in breast cancer development, about more than one-third of ERα-positive patients display de novo resistance or develop resistance over time to such existing therapies. Several mechanisms have been described to explain resistance to such hormone therapies. For example, hypersensitivity of ERα to low estrogen level in treatment with aromatase inhibitors, the switch of tamoxifen effects from antagonist to agonist effects in tamoxifen treatments or multiple growth factor receptor signaling pathways. Acquired mutations in ERα occurring after initiation of hormone therapies may also play a role in treatment failure and cancer progression. Certain mutations in ERα, particularly those identified in the Ligand Binding Domain (LBD), result in the ability to bind to DNA in the absence of ligand and confer hormone independence in cells harboring such mutant receptors.

Most of the endocrine therapy resistance mechanisms identified rely on ERα-dependent activity. One of the new strategies to counterforce such resistance is to shut down the ERα signaling by removing ERα from the tumor cells using Selective Estrogen Receptors Degraders (SERDs). Clinical and preclinical data showed that a significant number of the resistance pathways can be circumvented by the use of SERDs.

There is still a need to provide SERDs with good degradation efficacy.

Documents WO2017/140669 and WO2018/091153 disclose some substituted 6,7-dihydro-5H-benzo[7]annulene compounds and substituted N-(3-fluoropropyl)-pyrrolidine derivatives useful as SERDs. However, as detailed hereafter, such compounds do not display an optimized pharmacological profile, especially regarding the limitation of CYP3A4 induction.

The inventors have now found novel compounds able to selectively antagonize and degrade the estrogen receptors (SERDs compounds), for use in cancer treatment.

Disclosed herein are compounds of the formula (I), or pharmaceutically acceptable salts thereof:

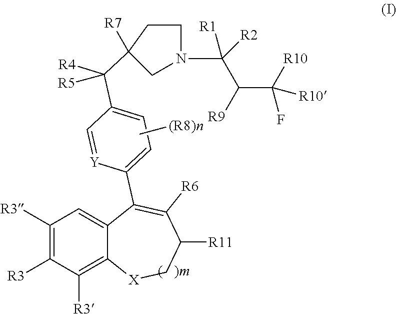

wherein
R1 and R2 independently represent a hydrogen atom or a deuterium atom;
R3 represents a hydrogen atom, a —COOH group or a —OH group;
R3′ and R3″ independently represent a hydrogen atom, a methyl group, a methoxy group, a chlorine atom, a fluorine atom, or a cyano group;
R4 and R5 independently represent a hydrogen atom, a fluorine atom, a —NH₂ group, a (C₁-C₃)alkyl group such as a methyl group, a (C₁-C₃)alkoxy group such as a methoxy group or an ethoxy group, or a —OH group; or R4 and R5 together form an oxo group or R4 and R5 together form a ═NOCH₃ group or a (C₃-C₅)cycloalkyl group with the carbon atom to which they are attached;
R7 represents a hydrogen atom, a methyl group, a —OH group or a fluorine atom; or alternatively R4 and R7 together form a cyclopropyl group together with the bond to which they are attached, that gives with the adjacent pyrrolidine group an azaspiro[2.4]heptane;
R6 represents a group selected from:
   a phenyl group, said phenyl group being optionally substituted by 1 to 3 substituents independently selected from a halogen atom; a (C₁-C₆)alkyl group optionally substituted with a cyano group or a —OH group; a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group;
   a fused phenyl group, selected from phenyl groups fused with a (C₃-C₆)cycloalkyl, which (C₃-C₆)cycloalkyl ring optionally comprises an unsaturation and wherein the fused phenyl is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
   a bicyclic group comprising 5 to 12 carbon atoms, optionally comprising 1 to 2 unsaturations; optionally substituted with 1 to 4 substituents independently selected from: a fluorine atom, a —OH group, a (C₁-C₃)alkyl group, a (C₁-C₃)fluoroalkyl group, a (C₁-C₃)alkoxy group, a (C₁-C₃)fluoroalkoxy group and an oxo group;
   a heteroaryl group comprising 2 to 9 carbon atoms and comprising from 1 to 3 heteroatoms independently selected from oxygen, nitrogen and sulfur, and at least 5 atoms including carbon atoms and heteroatoms, such as a pyridyl group, a pyridone group or a pyrrolyl group, said heteroaryl group being optionally substituted with 1 to 3 substituents independently selected from a halogen atom, a (C₁-C₆)alkyl group, a (C₁-C₆)fluoroalkyl group, a (C₁-C₆)alkoxy group, a (C₁-C₆)fluoroalkoxy group, a cyano group, a carbamoyl group and a —OH group;
   a cycloalkyl group comprising 3 to 7 carbon atoms, said cycloalkyl group being saturated or partially saturated and being optionally substituted with 1 to 4 substituents independently selected from:
      a fluorine atom, a —OH group, a (C₁-C₃)alkyl group, a (C₁-C₃)fluoroalkyl group, a (C₁-C₃)alkoxy group, a (C₁-C₃)fluoroalkoxy group, an oxo group,
      a (C₃-C₆)cycloalkyl group and a phenyl group, said (C₃-C₆)cycloalkyl or phenyl groups being optionally substituted with one or two halogen atom(s) or (C₁-C₃)alkyl group(s);
   a (C₃-C₆)cycloalkyl(C₁-C₃)alkyl group, optionally substituted on the cycloalkyl with 1 to 4 substituents independently selected from: a fluorine atom, a —OH group, a (C₁-C₄)alkyl group, a (C₁-C₃)fluoroalkyl group, a (C₁-C₃)fluoroalkoxy group and an oxo group;
   a 4 to 7 membered-heterocycloalkyl group comprising 1 or 2 heteroatoms independently selected from oxygen, nitrogen and sulfur, such as a tetrahydropyranyl group or a tetrahydrofuranyl, said heterocycloalkyl group being saturated or partially saturated and being optionally substituted with 1 to 3 substituents independently selected from: a fluorine atom, a (C₁-C₃)alkyl group, a (C₁-C₃)fluoroalkyl group, a (C₁-C₃)fluoroalkoxy group, an oxo group, a (C₁-C₃)alkoxy group and a —OH group;
   a (C₁-C₆)alkyl group, such as an isobutyl group or an ethylbutyl group, said alkyl group being optionally substituted with 1 to 4 substituents independently selected from: a fluorine atom, a (C₁-C₃)alkoxy group, a (C₁-C₃)fluoroalkoxy group and a —OH group; and
   a phenyl(C₁-C₂)alkyl group, said phenyl group being optionally substituted with 1 to 3 substituents independently selected from a halogen atom; a (C₁-C₃)alkyl group; a (C₁-C₃)fluoroalkyl group; a (C₁-C₃)alkoxy group; a (C₁-C₃)fluoroalkoxy group; a cyano group; and a —OH group;
X represents —CH₂—, —O—, or —S—;
Y represents —CH═, —N═ or —CR″═, wherein R″ represents a (C₁-C₃)alkyl group or a halogen atom, such as a fluorine or a chlorine atom, a cyano group, or a (C₁-C₃)fluoroalkyl group, such as a trifluoromethyl;
R8 independently represents a (C₁-C₃)alkyl group, such as a methyl group, a halogen atom, such as a fluorine atom, a cyano group, or a (C₁-C₃)fluoroalkyl group, such as a trifluoromethyl;
R9 represents a hydrogen atom or a fluorine atom;
R10 and R10′ independently represent a hydrogen atom or a fluorine atom;
R11 represents a hydrogen atom, or a (C₁-C₃)alkyl group or a cyclopropyl;
n is 0, 1 or 2, and
m is 0 or 1.

The compounds of formula (I) can contain one or more asymmetric carbon atoms. They may therefore exist in the form of enantiomers.

The compounds of formula (I) may be present as well under tautomer forms.

The compounds of formula (I) may exist in the form of bases, acids, zwitterion or of addition salts with acids or bases. Hence, herein are provided compounds of formula (I) or pharmaceutically acceptable salts thereof.

These salts may be prepared with pharmaceutically acceptable acids or bases, although the salts of other acids or bases useful, for example, for purifying or isolating the compounds of formula (I) are also provided.

Among suitable salts of the compounds of formula (I), hydrochloride may be cited.

As used herein, the terms below have the following definitions unless otherwise mentioned throughout the instant specification:
a halogen atom: a fluorine, a chlorine, a bromine or an iodine atom, and in particular a fluorine and a chlorine atom;
an oxo: a “═O” group;
an alkyl group: a linear or branched saturated hydrocarbon-based aliphatic group comprising, unless otherwise mentioned, from 1 to 6 carbon atoms (noted “(C₁-C₆)alkyl”). By way of examples, mention may be made of, but not limited to: methyl, ethyl, propyl, isopropyl, butyl, isobutyl, sec-butyl, tert-butyl, pentyl, isopentyl, hexyl and isohexyl groups, and the like;
a cycloalkyl group: a monocyclic alkyl group comprising, unless otherwise mentioned, from 3 to 7 carbon atoms, saturated or partially unsaturated and unsubstituted or substituted. By way of examples, mention may be made of, but not limited to: cyclopropyl, cyclobutyl, cyclopentyl, cyclobutenyl, cyclopentenyl, cyclohexyl, cyclohexenyl, cycloheptyl, cycloheptenyl, groups and the like, in particular a cyclopentyl, a cyclohexyl, a cycloheptyl, a cycloheptenyl, or a cyclohexenyl;
a cycloalkylalkyl group: an alkyl group substituted with a cyclic alkyl group as defined above. Mention may be made of, but not limited to: cyclobutylmethyl;
a heterocycloalkyl group: a 4 to 7-membered cycloalkyl group, saturated or partially unsaturated, comprising 1 to 2 heteroatoms independently selected from oxygen, nitrogen and sulfur, in particular being oxygen or nitrogen. By way of examples, mention may be made of, but not limited to: morpholinyl, piperazinyl, piperidinyl, pyrrolidinyl, aziridinyl, oxanyl, oxetanyl, tetrahydropyranyl, morpholinyl, tetrahydrofuranyl, oxepanyl, diazepanyl, dioxanyl, tetrahydropyranyl, and tetrahydrothiopyranyl. The heterocycloalkyl is advantageously tetrahydrofuranyl or tetrahydropyranyl.
a fluoroalkyl group: an alkyl group as previously defined where the alkyl group is substituted with at least one fluorine atom. In other terms, at least one hydrogen atom of the alkyl group is replaced by a fluorine atom. By way of example, mention may be made of —CH₂F, —CHF₂, —CH₂CHF₂, —CH₂CH₂F and the like. When all the hydrogen atoms belonging to the alkyl group are replaced by fluorine atoms, the fluoroalkyl group can be named perfluoroalkyl group. By way of example, mention may be made of trifluoromethyl group or trifluoroethyl group and the like;
an alkoxy group: an —O-alkyl group where the alkyl group is as previously defined. By way of examples, mention may be made of, but not limited to: methoxy, ethoxy, propoxy, isopropoxy, linear, secondary or tertiary butoxy, isobutoxy, pentoxy or hexoxy groups, and the like;
a fluoroalkoxy group: an —O-alkyl group where the alkyl group is as previously defined and where the alkyl group is substituted with at least one fluorine atom. In other terms, at least one hydrogen atom of the alkyl group is replaced by a fluorine atom. By way of example, mention may be made of —OCH₂F, —OCHF₂, —OCH₂CH₂F and the like. When all the hydrogen atoms belonging to the alkyl group are replaced by fluorine atoms, the fluoroalkoxy group can be named perfluoroalkoxy group. By way of example, mention may be made of trifluoromethoxy group and the like;
a (C₁-C₄)alkylthio group also named a (C₁-C₄)alkylsulfanyl group: a —S-alkyl group where the alkyl group is as previously defined. By way of examples, mention may be made of, but not limited to: methylthio, ethylthio, propylthio, isopropylthio, linear, secondary or tertiary butylthio, isobutylthio, and the like;
a (C₁-C₄)alkylsulfonyl group: a —SO₂-alkyl group where the alkyl group is as previously defined. By way of examples, mention may be made of, but not limited to —SO₂CH₃, —SO₂CH₂CH₃ and the like;
a (C₁-C₄)fluoroalkylthio group also named a (C₁-C₄)fluoroalkylsulfanyl group: a —S-fluoroalkyl group where the fluoroalkyl group is as previously defined. By way of examples, mention may be made of, but not limited to: fluoromethylthio, difluoromethylthio, trifluoromethylthio and the like;
a fused phenyl: a bicyclic radical comprising from 7 to 10 carbon atoms and that contains a phenyl moiety. Said phenyl moiety may be fused to a (C₃-C₆)cycloalkyl group, i.e. the phenyl moiety may share a bond with said (C₃-C₆)cycloalkyl group. The fused phenyl group may be bound to the rest of the molecule by its phenyl moiety. It may be substituted. Examples are, but are not limited to indanyl, bicyclo[4.2.0]octa-1(6),2,4-trienyl, tetrahydronaphthalenyl and the like;
a heteroaryl group: a cyclic 5 to 10-membered aromatic group containing between 2 and 9 carbon atoms and containing between 1 and 3 heteroatoms, such as nitrogen, oxygen or sulfur. Such nitrogen atom may be substituted with an oxygen atom in order to form a —N—O bond. Such —N—O bond can be in a form of a N-oxide (—N⁺—O⁻). Said heteroaryl group may be monocyclic or bicyclic. By way of examples of heteroaryl groups, mention may be made of, but not limited to: thiophene, furan, thiadiazole, thiazole, imidazole, pyridazine, triazine, pyrazine, oxadiazole, pyrazole, isothiazole, oxazole, isoxazole, pyridine, pyrimidine, benzotriazole, benzoxazole, pyrrolo[2,3-b]pyridine, benzimidazole, benzoxadiazole, benzothiazole, benzothiadiazole, benzofuran, indole, isoquinoline, indazole, benzisoxazole, benzisothiazole, pyridone groups and the like. The heteroaryl group is advantageously pyridine, pyrrole, imidazole, pyrazine, furane, thiazole, pyrazole, thiadiazole, pyridazine, pyridone and pyrimidine, and more particularly pyridine, pyridone and pyrrole;
a bicyclic group, generally comprising 5 to 12 carbon atoms, is a hydrocarbon group selected from groups comprising two rings connected through:
a single common atom: a “spirobicyclic ring”. Such spiro bicyclic alkyl generally comprises 5 to 11 carbon atoms referring to a “spiro(C₅-C₁₁)bicyclic ring”. The rings may be saturated or partially unsaturated. Such spirobicyclic ring may be unsubstituted or substituted, in particular by at least one (C₁-C₃)alkyl group such as methyl or a fluorine. By way of examples of spiro(C₅-C₁₁)bicyclic ring as for the definition of R6, mention may be made of, but not limited to: spiro[2.3]hexane, spiro[3.3]heptane, spiro[3.3]heptene, spiro[2.5]octane and 7-azaspiro[3.5]nonane. The spiro(C₅-C₁₁)bicyclic ring is advantageously spiro[3.3]heptane or spiro[3.3]heptene still for the R6 group.
two common atoms: In that case the bicyclic group comprises 7 to 12 carbon atoms and optionally comprises 1 to 2 unsaturations. By way of examples of such bicyclic groups, mention may be made of, but not limited to: cis-1,3a,4,5,6,6a-hexahydropentalenyl group, bicyclo[3.1.0]hexan-1-yl and bicyclo[4.1.0]heptanyl.
three or more common atoms: In that case the bicyclic group comprises 6 to 10 carbon atoms, such bicyclic group may be referred to as a “bridged (C₆-C₁₀)cycloalkyl” group, the rings share three or more atoms and the bridge contains at least one atom, for example 1, 2 or 3 atoms and preferentially 1 atom. By way of examples of such bridged cycloalkyl groups, mention may be made of, but not limited to bicyclo[3.2.1]octan-3-yl and bicyclo[2.2.1]heptan-2-yl.
a zwitterion means: a globally neutral molecule with a positive and a negative electrical charge and having an acidic group and a basic group.

In another embodiment, in the compounds of formula (I) as defined above, R1 and R2 are a hydrogen atom.

In another embodiment, in the compounds of formula (I) as defined above, R3 is a —COOH group or a —OH group, more particularly R3 is a —COOH group.

In another embodiment, in the compounds of formula (I) as defined above, when m is 1 then X represents —CH₂— or —O—.

In another embodiment, in the compounds of formula (I) as defined above, X represents —CH₂— or —O—, in particular X represents —CH₂—.

In another embodiment, in the compounds of formula (I) as defined above, R4 and R5 independently represent a hydrogen atom or a fluorine atom, more particularly R4 and R5 both represent a hydrogen atom or R4 and R5 both represent a fluorine atom.

In another embodiment, in the compounds of formula (I) as defined above, R4 and R5 both represent a hydrogen atom.

In another embodiment, in the compounds of formula (I) as defined above, R7 represents a hydrogen atom or a fluorine atom, more particularly a hydrogen atom.

In another embodiment, in the compounds of formula (I) as defined above:
R4 and R5 independently represent a hydrogen atom, a —NH₂ group, a (C₁-C₃)alkyl group such as a methyl group, a (C₁-C₃)alkoxy group such as a methoxy group or an ethoxy group, or a —OH group; or R4 and R5 together form an oxo group or R4 and R5 together form a =NOCH₃ group or a (C₃-C₅)cycloalkyl group with the carbon atom to which they are attached, and
R7 represents a hydrogen atom, a methyl group or a —OH group;
   or alternatively R4 and R7 together form a cyclopropyl group together with the bond to which they are attached, that gives with the adjacent pyrrolidine group an azaspiro[2.4]heptane.

In another embodiment, in the compounds of formula (I) as defined above, R6 represents a group selected from:
a phenyl group, said phenyl group being optionally substituted by 1 to 3 substituents independently selected from a halogen atom; a (C₁-C₆)alkyl group optionally substituted with a cyano group or a —OH group; a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group;
a heteroaryl group comprising 2 to 9 carbon atoms and comprising from 1 to 3 heteroatoms independently selected from oxygen, nitrogen and sulfur, and at least 5 atoms including carbon atoms and heteroatoms, such as a pyridyl group, a pyridone group or a pyrrolyl group, said heteroaryl group being optionally substituted with 1 to 3 substituents independently selected from a halogen atom, a (C₁-C₆)alkyl group, a (C₁-C₆)fluoroalkyl group, a (C₁-C₆)alkoxy group, a (C₁-C₆)fluoroalkoxy group, a cyano group, a carbamoyl group and a —OH group; and
a cycloalkyl group comprising 3 to 7 carbon atoms, said cycloalkyl group being saturated or partially saturated and being optionally substituted with 1 to 4 substituents independently selected from: a fluorine atom, a —OH group, a (C₁-C₃)alkyl group, a (C₁-C₃)fluoroalkyl group, a (C₃-C₆)cycloalkyl group, a (C₁-C₃)alkoxy group, a (C₁-C₃)fluoroalkoxy group, an oxo group and a phenyl group optionally substituted with a halogen atom or a (C₁-C₃)alkyl group.

In another embodiment, in the compounds of formula (I) as defined above, R6 represents a phenyl group, said phenyl group being optionally substituted with 1 to 3 substituents independently selected from a halogen atom, a (C₁-C₆)alkyl group and a (C₁-C₆)fluoroalkyl group; more particularly selected from: a fluorine atom, a chlorine atom, a methyl group and a trifluoromethyl group. In such embodiment, in particular, at least one of the substituents of said phenyl group comprises a halogen atom.

In another embodiment, in the compounds of formula (I) as defined above, R6 represents a pyridyl group, said pyridyl group being optionally substituted with 1 to 3 substituents independently selected from a (C₁-C₆)alkoxy group, more particularly a methoxy group.

In another embodiment, in the compounds of formula (I) as defined above, R6 represents a saturated or partially saturated cyclohexyl group, more particularly a cyclohexyl group or a cyclohexenyl group, said saturated or partially saturated cyclohexyl group being optionally substituted with 1 to 4 substituents independently selected from a (C₁-C₃)alkyl group, more particularly with 1 to 4 methyl group(s).

In another embodiment, in the compounds of formula (I) as defined above, R3′ and R3″ both represent a hydrogen atom.

In another embodiment, in the compounds of formula (I) as defined above, n is 0.

In another embodiment, in the compounds of formula (I) as defined above, Y represents —CH═.

In another embodiment, in the compounds of formula (I) as defined above, R9 represents a hydrogen atom.

In another embodiment, in the compounds of formula (I) as defined above, R10 and R10′ both represent a hydrogen atom; or one of R10 and R10′ represents a hydrogen atom and the other a fluorine atom.

In another embodiment, in the compounds of formula (I) as defined above, R10 and R10′ both represent a hydrogen atom.

In another embodiment, in the compounds of formula (I) as defined above, R11 represents a hydrogen atom or a methyl group, more particularly a hydrogen atom.

In another embodiment, in the compounds of formula (I) as defined above, m is 1.

In another embodiment, in the compounds of formula (I) as defined above, R3 is a —COOH group and R6 is a phenyl group comprising one, two or three substitutions independently selected from a chlorine atom, a fluorine atom, a chlorine atom, a methyl group and a trifluoromethyl group, at least one of the substitutions comprising a halogen atom. In such embodiment, R3′ and R3″ are in particular hydrogen atoms. Still in such embodiment, R1, R2, R4, R5, R7, R9 and R11 are hydrogen atoms. In such embodiment, both R10 and R10′ are hydrogen atoms or one of R10 and R10′ is a hydrogen atom and the other is a fluorine atom. Still in such embodiment, Y is —CH═, m is equal to 1 and n is equal to 0. Still in such embodiment, X is a —CH₂— group.

Among the compounds of formula (I) described herein, mention may be made in particular of the following compounds or a pharmaceutically acceptable salt thereof, in particular hydrochloride salt thereof:
8-(2,4-dichlorophenyl)-9-(4-((1-(3-fluoropropyl)pyrrolidin-3-yl)methyl)phenyl)-6,7-dihydro-5H-benzo[7]annulene-3-carboxylic acid, (S) isomer (1),
8-(2-chloro-4-fluorophenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (2),
8-(4,4-dimethylcyclohexyl)-9-(4-((1-(3-fluoropropyl)pyrrolidin-3-yl)methyl)phenyl)-6,7-dihydro-5H-benzo[7]annulene-3-carboxylic acid, Isomer 1 (3),
9-(4-((1-(3,3-difluoropropyl)pyrrolidin-3-yl)methyl)phenyl)-8-(4-fluoro-2-methylphenyl)-6,7-dihydro-5H-benzo[7]annulene-3-carboxylic acid hydrochloride, Isomer 1 (4),
9-(4-((1-(3,3-difluoropropyl)pyrrolidin-3-yl)methyl)phenyl)-8-(4-fluoro-2-(trifluoromethyl)phenyl)-6,7-dihydro-5H-benzo[7]annulene-3-carboxylic acid hydrochloride, Isomer 1 (5),
8-(3-chloro-2-methylphenyl)-9-(4-((1-(3,3-difluoropropyl)pyrrolidin-3-yl)methyl)phenyl)-6,7-dihydro-5H-benzo[7]annulene-3-carboxylic acid hydrochloride, Isomer 1 (6),
4-(2,4-dichlorophenyl)-5-(4-((1-(3-fluoropropyl)pyrrolidin-3-yl)methyl)phenyl)-2,3-dihydrobenzo[b]oxepine-8-carboxylic acid hydrochloride, Isomer 1 (7),
4-(2,4-dichlorophenyl)-5-(4-((1-(3-fluoropropyl)pyrrolidin-3-yl)methyl)phenyl)-2,3-dihydrobenzo[b]oxepine-8-carboxylic acid hydrochloride, Isomer 2 (8),
8-(2,4-dichlorophenyl)-9-(4-((1-(3,3-difluoropropyl)pyrrolidin-3-yl)methyl)phenyl)-6,7-dihydro-5H-benzo[7]annulene-3-carboxylic acid, Isomer 1 (9),
8-(2,4-difluorophenyl)-9-(4-((1-(3,3-difluoropropyl)pyrrolidin-3-yl)methyl)phenyl)-6,7-dihydro-5H-benzo[7]annulene-3-carboxylic acid, Isomer 1 (10),
8-(4,4-dimethylcyclohex-1-en-1-yl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (11),
9-(4-((1-(3-fluoropropyl)pyrrolidin-3-yl)methyl)phenyl)-8-(2-methyl-3-(trifluoromethyl)phenyl)-6,7-dihydro-5H-benzo[7]annulene-3-carboxylic acid hydrochloride, Isomer 1 (12),
8-(3-chloro-2-(trifluoromethyl)phenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (13),
9-(4-((1-(3-fluoropropyl)pyrrolidin-3-yl)methyl)phenyl)-8-(6-methoxypyridin-3-yl)-6,7-dihydro-5H-benzo[7]annulene-3-carboxylic acid, Isomer 1 (14),
8-(3-chloro-2-methylphenyl)-9-(4-((1-(3-fluoropropyl)pyrrolidin-3-yl)methyl)phenyl)-6,7-dihydro-5H-benzo[7]annulene-3-carboxylic acid, Isomer 1 (15),
8-(3-chloro-2-fluorophenyl)-9-(4-((1-(3-fluoropropyl)pyrrolidin-3-yl)methyl)phenyl)-6,7-dihydro-5H-benzo[7]annulene-3-carboxylic acid, Isomer 1 (16),
8-(4-fluoro-2-methylphenyl)-9-(4-((1-(3-fluoropropyl)pyrrolidin-3-yl)methyl)phenyl)-6,7-dihydro-5H-benzo[7]annulene-3-carboxylic acid, Isomer 1 (17),
8-(4-fluoro-2-(trifluoromethyl)phenyl)-9-(4-((1-(3-fluoropropyl)pyrrolidin-3-yl)methyl)phenyl)-6,7-dihydro-5H-benzo[7]annulene-3-carboxylic acid, Isomer 1 (18),
8-(2,4-difluorophenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (19),
8-(3-fluoro-2-methylphenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (20),
8-(2-chloro-3-fluorophenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (21),
8-(2,4-dichlorophenyl)-9-(4-((3-fluoro-1-(3-fluoropropyl)pyrrolidin-3-yl)methyl)phenyl)-6,7-dihydro-5H-benzo[7]annulene-3-carboxylic acid, 2,2,2-trifluoroacetic acid, Isomer 2 (22),
8-(2,4-dichlorophenyl)-9-(4-((3-fluoro-1-(3-fluoropropyl)pyrrolidin-3-yl)methyl)phenyl)-6,7-dihydro-5H-benzo[7]annulene-3-carboxylic acid, 2,2,2-trifluoroacetic acid, Isomer 1 (23),
8-(2,4-dichlorophenyl)-9-(4-((1-(3-fluoropropyl)pyrrolidin-3-yl)methyl)phenyl)-6,7-dihydro-5H-benzo[7]annulene-3-carboxylic acid, (R) Isomer (24),
3-(2,4-dichlorophenyl)-4-(4-((1-(3-fluoropropyl)pyrrolidin-3-yl)methyl)phenyl)-2H-thiochromene-7-carboxylic acid hydrochloride, Isomer 1 (25),
8-(2,4-dichlorophenyl)-9-(4-(difluoro(1-(3-fluoropropyl)pyrrolidin-3-yl)methyl)phenyl)-6,7-dihydro-5H-benzo[7]annulene-3-carboxylic acid, Isomer 1 (26),
8-(2-chloro-4-fluorophenyl)-9-(4-((1-(3,3-difluoropropyl)pyrrolidin-3-yl)methyl)phenyl)-6,7-dihydro-5H-benzo[7]annulene-3-carboxylic acid hydrochloride, Isomer 1 (27),
9-(4-((1-(3,3-difluoropropyl)pyrrolidin-3-yl)methyl)phenyl)-8-(2-methyl-3-(trifluoromethyl)phenyl)-6,7-dihydro-5H-benzo[7]annulene-3-carboxylic acid hydrochloride, Isomer 1 (28),
8-(2,4-dichlorophenyl)-9-(4-((1-(3-fluoropropyl)pyrrolidin-3-yl)methyl)phenyl)-6,7-dihydro-5H-benzo[7]annulene-3-ol hydrochloride, Isomer 1 (29),
9-(4-((1-(3-fluoropropyl)pyrrolidin-3-yl)methyl)phenyl)-8-(3-methyl-2-(trifluoromethyl)phenyl)-6,7-dihydro-5H-benzo[7]annulene-3-carboxylic acid, Isomer 1 (30),
8-(2,4-dichlorophenyl)-9-(4-((1-(3,3-difluoropropyl)pyrrolidin-3-yl)methyl)phenyl)-6,7-dihydro-5H-benzo[7]annulen-3-ol hydrochloride, Isomer 1 (31),
9-(4-((1-(3,3-difluoropropyl)pyrrolidin-3-yl)methyl)phenyl)-8-(4-fluoro-2,6-dimethylphenyl)-6,7-dihydro-5H-benzo[7]annulene-3-carboxylic acid hydrochloride, Isomer 1 (32),
9-(4-((1-(3,3-difluoropropyl)pyrrolidin-3-yl)methyl)phenyl)-8-(2-(trifluoromethyl)phenyl)-6,7-dihydro-5H-benzo[7]annulene-3-carboxylic acid (33),
9-(4-((1-(3,3-difluoropropyl)pyrrolidin-3-yl)methyl)phenyl)-8-(3-methyl-2-(trifluoromethyl)phenyl)-6,7-dihydro-5H-benzo[7]annulene-3-carboxylic acid hydrochloride, Isomer 1 (34),
8-(4-fluoro-2,6-dimethylphenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (35),
6-(2,4-dichlorophenyl)-5-(4-((1-(3-fluoropropyl)pyrrolidin-3-yl)methyl)phenyl)-7,8-dihydronaphthalene-2-carboxylic acid hydrochloride, Isomer 1 (36),
8-(4-chlorophenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (37),
9-(4-((1-(3-fluoropropyl)pyrrolidin-3-yl)methyl)phenyl)-8-(4-(trifluoromethyl)phenyl)-6,7-dihydro-5H-benzo[7]annulene-3-carboxylic acid hydrochloride, Isomer 1 (38),
9-(4-((1-(3-fluoropropyl)pyrrolidin-3-yl)methyl)phenyl)-8-(3-(trifluoromethyl)phenyl)-6,7-dihydro-5H-benzo[7]annulene-3-carboxylic acid hydrochloride, Isomer 1 (39),
8-(3-chlorophenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (40),
9-(4-((1-(3-fluoropropyl)pyrrolidin-3-yl)methyl)phenyl)-7-methyl-8-(4-(trifluoromethyl)phenyl)-6,7-dihydro-5H-benzo[7]annulene-3-carboxylic acid, Isomer 1 (41),
9-(4-((1-(3-fluoropropyl)pyrrolidin-3-yl)methyl)phenyl)-7-methyl-8-(4-(trifluoromethyl)phenyl)-6,7-dihydro-5H-benzo[7]annulene-3-carboxylic acid, Isomer 2 (42),
4-(2,4-dichlorophenyl)-5-(4-((1-(3-fluoropropyl)pyrrolidin-3-yl)methyl)phenyl)-2,3-dihydrobenzo[b]thiepin-8-ol, Isomer 1 (43),
4-(2-chlorophenyl)-5-(4-((1-(3-fluoropropyl)pyrrolidin-3-yl)methyl)phenyl)-2,3-dihydrobenzo[b]thiepin-8-ol, Isomer 1 (44).

Another embodiment is a compound selected from the above list, or a pharmaceutically acceptable salt thereof, for use in therapy, especially as an inhibitor and degrader of estrogen receptors.

Another embodiment is a compound selected from the above list, or a pharmaceutically acceptable salt thereof, for use in the treatment of cancer, especially breast cancer.

Another embodiment is a method of inhibiting and degrading estrogen receptors, comprising administering to a subject in need thereof, in particular a human, a therapeutically effective amount of a compound selected from the above list, or a pharmaceutically acceptable salt thereof.

Another embodiment is a method of treating ovulatory dysfunction, cancer, endometriosis, osteoporosis, benign prostatic hypertrophy or inflammation, comprising administering to a subject in need thereof, in particular a human, a therapeutically effective amount of a compound selected from the above list, or a pharmaceutically acceptable salt thereof.

Another embodiment is a method of treating cancer, comprising administering to a subject in need thereof, in particular a human, a therapeutically effective amount of a compound selected from the above list, or a pharmaceutically acceptable salt thereof.

Another embodiment is a pharmaceutical composition comprising as active principle an effective dose of a compound selected from the above list, or a pharmaceutically acceptable salt thereof, and also at least one pharmaceutically acceptable excipient.

The inventors surprisingly highlighted that compounds of formula (I) display an improved pharmacological profile, especially as CYP3A4 non-inductors in other terms by minimizing CYP3A4 induction, which is not predictable from prior art, in particular in view of WO2017/140669 and WO2018/091153. The following illustrative part highlights this unexpected property in comparison to chemical compounds representative of the prior art as well as in connection to a large number of compounds of formula (I) of the present invention.

Cytochrome P450 isoform 3A4 (CYP3A4) is involved in the metabolism of about 30% of clinically used drugs from almost all therapeutic categories. The evaluation of CYP3A4 induction potential is thus important for the prediction of drug-drug interactions due to CYP3A4 overexpression. More precisely, low CYP3A4 induction indicates limited undesired drug-drug interactions. In particular, the measurement of the CYP3A4 induction potential of a compound is part of its overall ADME (Absorption, Distribution, Metabolism, Excretion) evaluation.

Another embodiment is thus a compound selected from the above list, or a pharmaceutically acceptable salt thereof, for use in therapy, especially as an inhibitor and degrader of estrogen receptors, which simultaneously displays an improved pharmacological profile, especially demonstrating no CYP3A4 induction, or a weak or intermediary CYP3A4 induction.

As used herein, the term “no CYP3A4 induction, or weak or intermediary CYP3A4 induction” with respect to a compound relates respectively to no extent of CYP3A4 induction, or a weak or moderate extent of CYP3A4 induction which is typically calculated from the results of the test “CYP3A4 induction potential for ADME profile evaluation” in comparison to the reference compound rifampicin and named “% Eₘₐₓ”. Such test is based, for example, on the quantification of CYP3A4 mRNA induction performed by RT-PCR analysis. Said % Eₘₐₓ may be calculated as detailed in the illustrative part under the following formula:

$$\% E max = \frac{{E}_{compound} - {E}_{vehicle control}}{{E}_{positive control} - {E}_{vehicle control}} \times 100 \%$$

E_{Compound}: relative fold mRNA expression following treatment with test compound,
E_{Positive control}: relative fold mRNA expression following reference inducer i.e., 10 μM rifampicin,
E_{Vehicle control}: relative fold mRNA expression following vehicle treatment i.e., 0.1% DMSO, and
E is the efficiency of the quantitative PCR.

Typically, compounds for which Eₘₐₓ is equal or less than 20% may be considered as compounds showing a weak CYP3A4 induction or no CYP3A4 induction (low risk of drug-drug interactions) and compounds for which Eₘₐₓ is comprised between 20% and 45% may be considered as compounds showing intermediary CYP3A4 induction (limited risk of drug-drug interactions).

Another embodiment is a compound selected from the above list, or a pharmaceutically acceptable salt thereof, for use in the treatment of cancer, especially breast cancer, which simultaneously displays an improved pharmacological profile, typically a low or limited risk of drug-drug interactions, especially demonstrating no CYP3A4 induction, or a weak or intermediary CYP3A4 induction.

Another embodiment is a method of inhibiting and degrading estrogen receptors comprising administering to a subject in need thereof, in particular a human, a therapeutically effective amount of a compound selected from the above list, or a pharmaceutically acceptable salt thereof, which displays an improved pharmacological profile, typically a low or limited risk of drug-drug interactions, especially demonstrating no CYP3A4 induction, or a weak or intermediary CYP3A4 induction.

Another embodiment is a method of treating ovulatory dysfunction, cancer, endometriosis, osteoporosis, benign prostatic hypertrophy or inflammation, comprising administering to a subject in need thereof, in particular a human, a therapeutically effective amount of a compound selected from the above list, or a pharmaceutically acceptable salt thereof, which displays an improved pharmacological profile, typically a low or limited risk of drug-drug interactions, especially demonstrating no CYP3A4 induction, or a weak or intermediary CYP3A4 induction.

Another embodiment is a method of treating cancer comprising administering to a subject in need thereof, in particular a human, a therapeutically effective amount of a compound selected from the above list, or a pharmaceutically acceptable salt thereof, which displays an improved pharmacological profile, typically a low or limited risk of drug-drug interactions, especially demonstrating no CYP3A4 induction, or a weak or intermediary CYP3A4 induction.

The compounds of the formula (I) can be prepared by the following processes.

The compounds of the formula (I) and other related compounds having different substituents are synthesized using techniques and materials described below or otherwise known by the skilled person in the art. In addition, solvents, temperatures and other reaction conditions presented below may vary as deemed appropriate to the skilled person in the art.

General below methods for the preparation of compounds of formula (I) optionally modified by the use of appropriate reagents and conditions for the introduction of the various moieties found in the formula (I) are described below.

The following abbreviations and empirical formulae are used:

| | |
|---|---|
| ADME | Absorption, Distribution, Metabolism and Excretion |
| AIBN | Azobisisobutyronitrile |
| MeCN | Acetonitrile |
| NH₄Cl | Ammonium chloride |
| CO | Carbon monoxide |
| DDI | Drug-Drug Interaction |
| BAST | Deoxo-Fluor |
| Cs₂CO₃ | Cesium carbonate |
| CYP3A4 | Cytochrome P450 (CYP) 3A4 |
| DBU | 1,8-Diazabicyclo[5.4.0]undec-7-ene |
| DCM | Dichloromethane |
| Et₂O | Diethyl ether |
| DIEA | Diisopropylethylamine |
| DMF | N,N-dimethylformamide |
| DMAP | Dimethylaminopyridine |
| DMSO | Dimethyl sulfoxide |
| Boc₂O | di-ter-butyldicarbonate |
| EtOH | Ethanol |
| EtOAc | Ethyl acetate |
| H₂ | Hydrogen |
| HCl | Hydrochloric acid |
| IsoPrOH | Isopropanol |
| LIHMDS | Lithium bis(trimethylsilyl)amide |
| LiOH | Lithium hydroxide |
| MeOH | Methanol |
| MgSO₄ | Magnesium sulfate |
| MeTHF | 2-Methyltetrahydrofuran |
| Pd/C | Palladium on carbon |
| KOAc | Potassium acetate |
| KHMDS | Potassium bis(trimethylsilyl)amide |
| K₂CO₃ | Potassium carbonate |
| NaBH₄ | Sodium borohydride |
| NaHCO₃ | Sodium bicarbonate |
| NaH | Sodium hydride |
| NaOH | Sodium hydroxide |
| Na₂SO₄ | Sodium sulfate |
| NaHSO₃ | Sodium bisulfate |
| SCX | Strong cation exchange |
| PCR | Polymerase Chain Reaction |
| Pd(dppf)Cl₂ | [1,1′-Bis(diphenylphosphino)ferrocene]dichloropalladium(II) |
| Pd(PPh₃)₂Cl₂ | bis (triphenylphosphine) palladium(II) dichloride |
| Pd₂(dba)₃ | tris(dibenzylideneacetone)dipalladium(0) |
| PhOK | Potassium phenolate |
| SFC | Supercritical Fluid Chromatography |
| TEA | Triethylamine |
| TFA | Trifluoroacetic acid |
| THF | Tetrahydrofuran |
| PPh₃ | Triphenylphosphine |
| RT | Room temperature |
| RT-PCR | Reverse Transcriptase PCR |

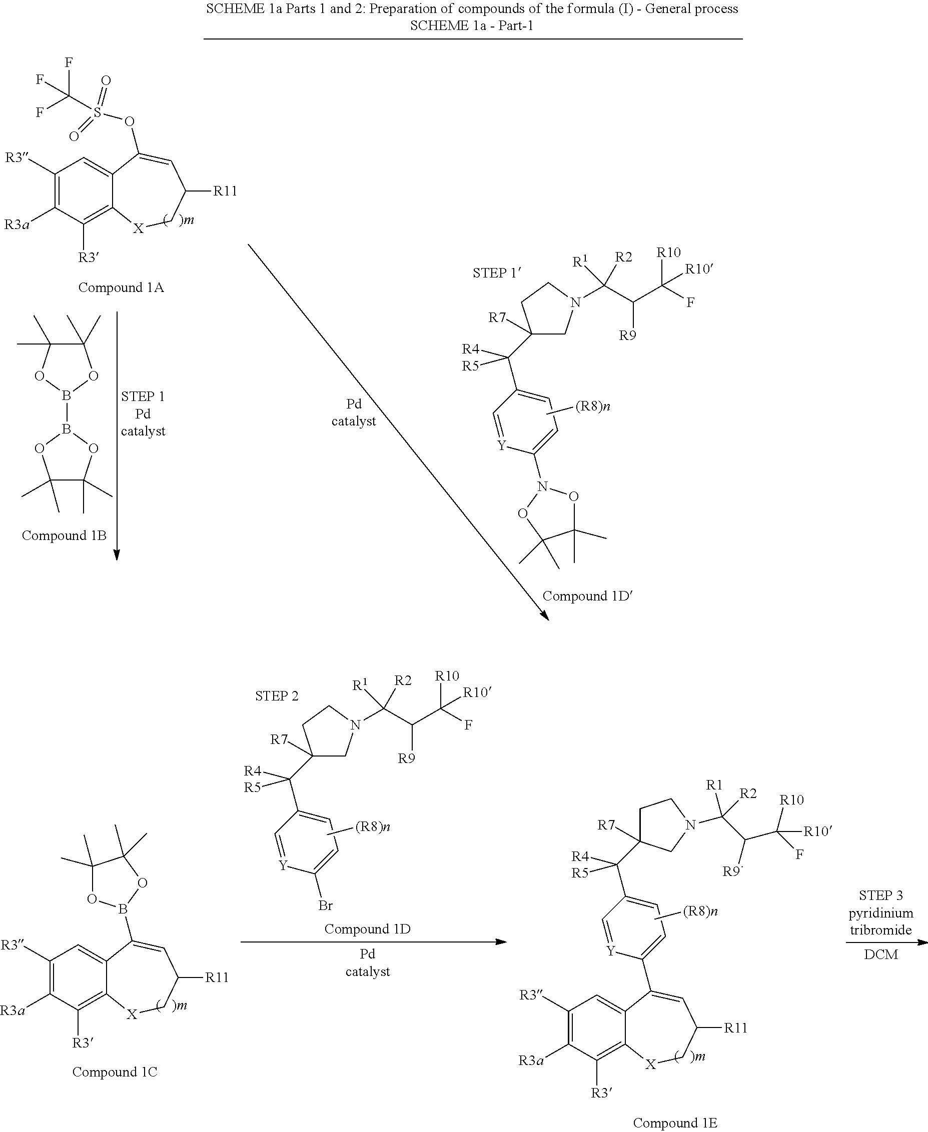

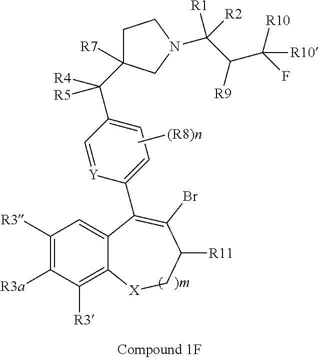

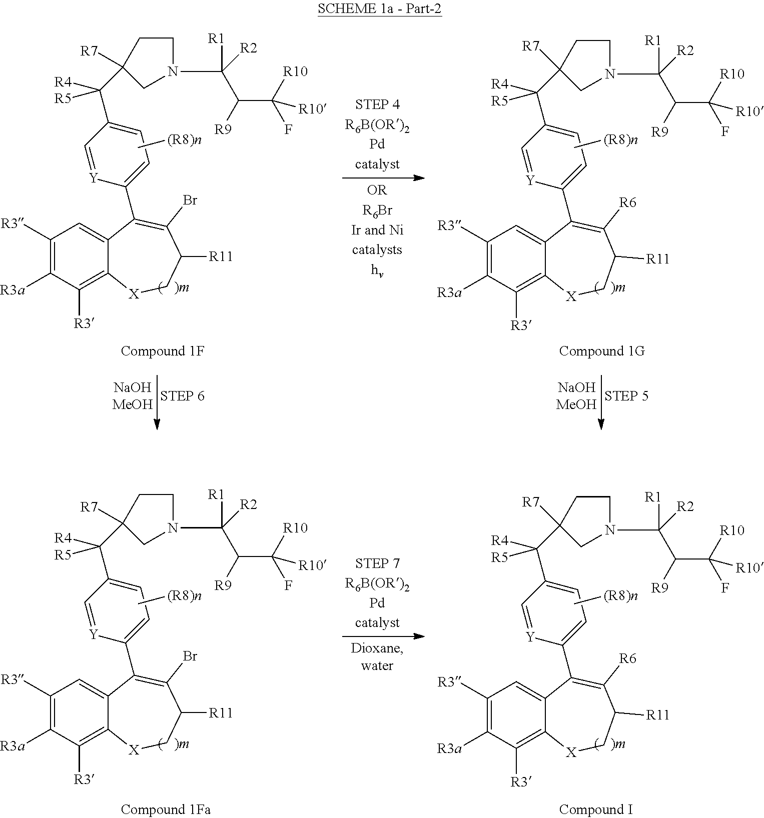

According to SCHEME 1a—Part-1 and Part-2, in which R3a is a hydrogen atom, a carboxylic ester such as COOMe, COOEt, or protected OH such as 0-pivaloyl, R1, R2, R3, R3′, R3″, R4, R5, R6, R7, R8, R9, R10, R10′, R11, n, m, X and Y are as defined above, compound 1A (prepared according to WO2017140669 when X═C), can be converted in STEP 1 to compound 1C by treatment with compound 1B in the presence of a palladium catalyst, for example bis(triphenylphosphine) palladium(II) dichloride Pd(PPh₃)₂Cl₂, and a phosphine such as triphenylphosphine in solution in toluene by heating up to reflux of solvent in presence of a base such as KOPh.

Compound 1C can be converted in STEP 2 to compound 1E by treatment with compound 1D in a Suzuki coupling reaction using for example [1,1′-bis(diphenylphosphino) ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

Alternatively, compound 1E can be obtained in STEP 1′ by Suzuki coupling between compound 1A and compound 1D′ using for example [1,1′-bis(diphenylphosphino) ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

Compound 1E, can be converted in STEP 3 to compound 1F by treatment for example with pyridinium tribromide in DCM or THF at room temperature.

This bromo derivative intermediate 1F can then be subjected in STEP 4 to a second Suzuki coupling with a suitable boronic reagent R6B(OR′)₂, wherein —B(OR′)₂ is a boronic acid or a pinacolate ester and R6 is defined as above, using for example Pd(dppf)Cl₂, complex with DCM, as catalyst, in a mixture of dioxane and water as solvent and in the presence of a base, for example Cs₂CO₃, at room temperature or by heating up to reflux to give compound 1G. When R6 is a substituted cycloalkene, heterocycloalkene or aliphatic ethylene, it may be reduced by hydrogenation with a catalyst such as Pd/C under hydrogen pressure (H₂) around 5 bars for example at temperature up to 70° C. to give the corresponding saturated compound 1G.

Alternatively, compound 1F can be subjected to a photocatalyzed coupling reaction with R6Br, where R6 is an alkyl group, a cycloalkyl or a spiro bicyclic alkyl as defined above, using catalysts such as (Ir[dF(CF₃)ppy]₂(dtbpy))PF₆ and nickel(II) chloride ethylene glycol dimethyl ether complex in presence of tris(trimethylsilyl)silane and bases such as 4,4′-di-tert-butyl-2,2′-bipyridine and sodium carbonate to give the corresponding compound 1G.

Compound 1G can be converted in STEP 5 to compound of formula (I) in presence of a source of hydroxide ions such as NaOH in solution in methanol (MeOH).

Compound 1F can be converted in STEP 6 to compound 1Fa in the presence of a source of hydroxide ions such as NaOH in solution in methanol (MeOH).

This compound 1Fa can be converted in STEP 7 to compound I through Suzuki conditions using a suitable boronic reagent R6B(OR′)₂, wherein —B(OR′)₂ is a boronic acid or a pinacolate ester and R6 is as above defined, using for example Pd(dppf)Cl₂, complex with DCM, as catalyst, in a mixture of dioxane and water as solvent and in the presence of a base, for example Cs₂CO₃, at room temperature or by heating up to reflux of solvents. When R6 is a substituted cycloalkene, heterocycloalkene or aliphatic ethylene, it may be reduced by hydrogenation with a catalyst, such as Pd/C under hydrogen (H₂) pressure around 5 bars, for example at temperature up to 70° C., to give the corresponding saturated compound I.

When R3a is COOMe, COOEt, or a protected OH such as O-pivaloyl, deprotection can be performed in STEP 5 by treatment with an aqueous solution of sodium hydroxide (NaOH) 2N or lithium hydroxide (LiOH) in MeOH. When R3 is COOH, extraction of the product can give the sodium salt of compound I. The acidification with an aqueous solution of HCl 2N to pH 6-7 can give the neutral form of compound I. The acidification with an aqueous solution of HCl 2N to pH 1-2 can give the hydrochloride salt of compound I. The purification using HPLC in presence of formic acid or trifluoroacetic acid in the eluent can give the formate or trifluoroacetate salt of compound I.

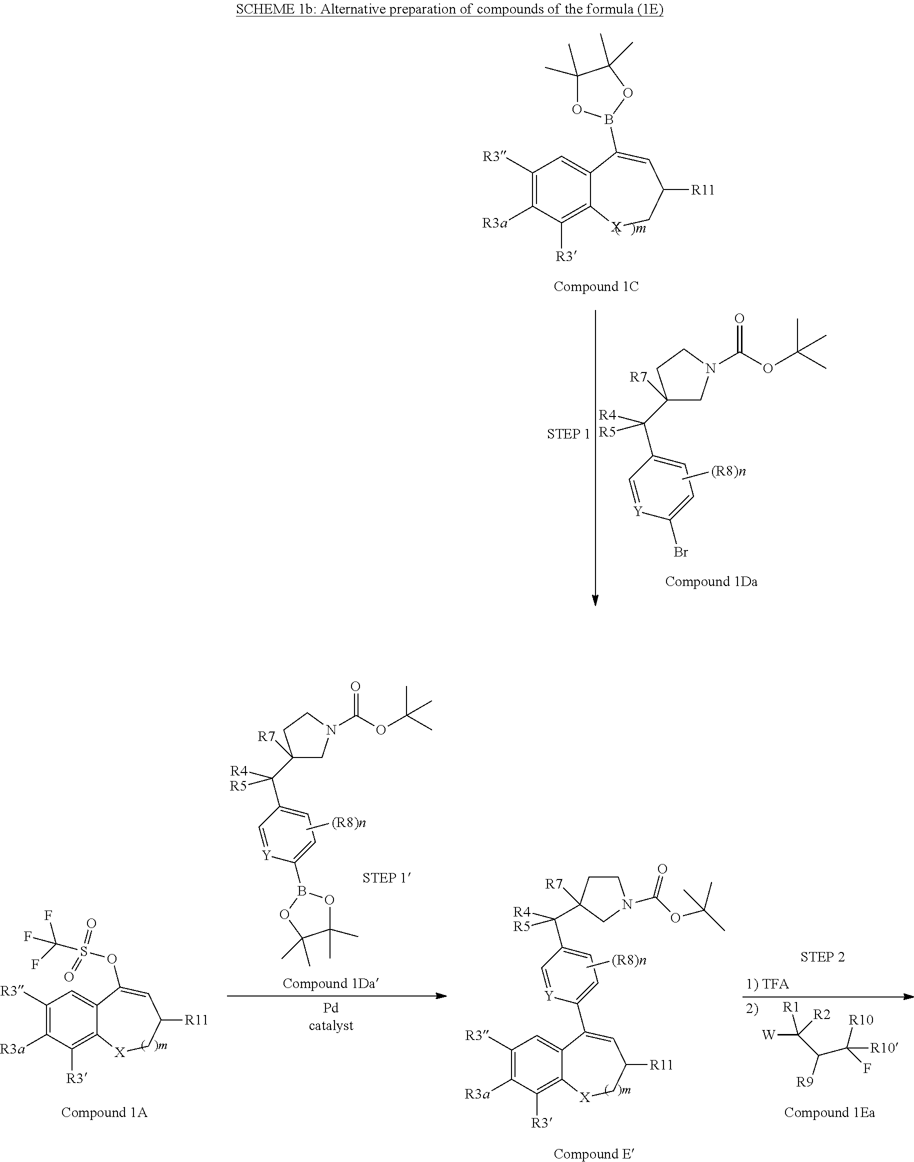

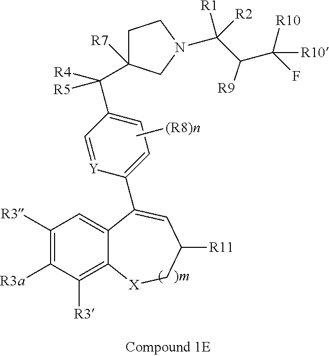

According to SCHEME 1b, in which R3a is a hydrogen atom, a carboxylic ester such as COOMe, COOEt or protected OH with O-pivaloyl for example, and R1, R2, R3, R3′, R3″, R4, R5, R7, R8, R9, R10, R10′, R11, n, m, X and Y are as defined above, compound 1E′ can be prepared in a Suzuki coupling reaction either between compounds 1C and 1Da in STEP 1 or between compounds 1A and 1Da′ in STEP 1′ in the presence of a palladium catalyst, for example [1,1′-bis(diphenylphosphino)ferrocene]dichloropalladium(II) (Pd(dppf)Cl₂), complex with DCM, in a mixture of dioxane and water and in the presence of a base, for example cesium carbonate (Cs₂CO₃), by heating up to reflux of solvent.

Compound 1E′ can be converted in STEP 2 to compound 1E in presence of TFA followed by the treatment with compound 1Ea, wherein W is Cl, Br or I or OS₂R with R=CH₃, PhMe, CF₃ or CF₂CF₂CF₂CF₃ in presence of a base such as potassium carbonate in DMF as a solvent.

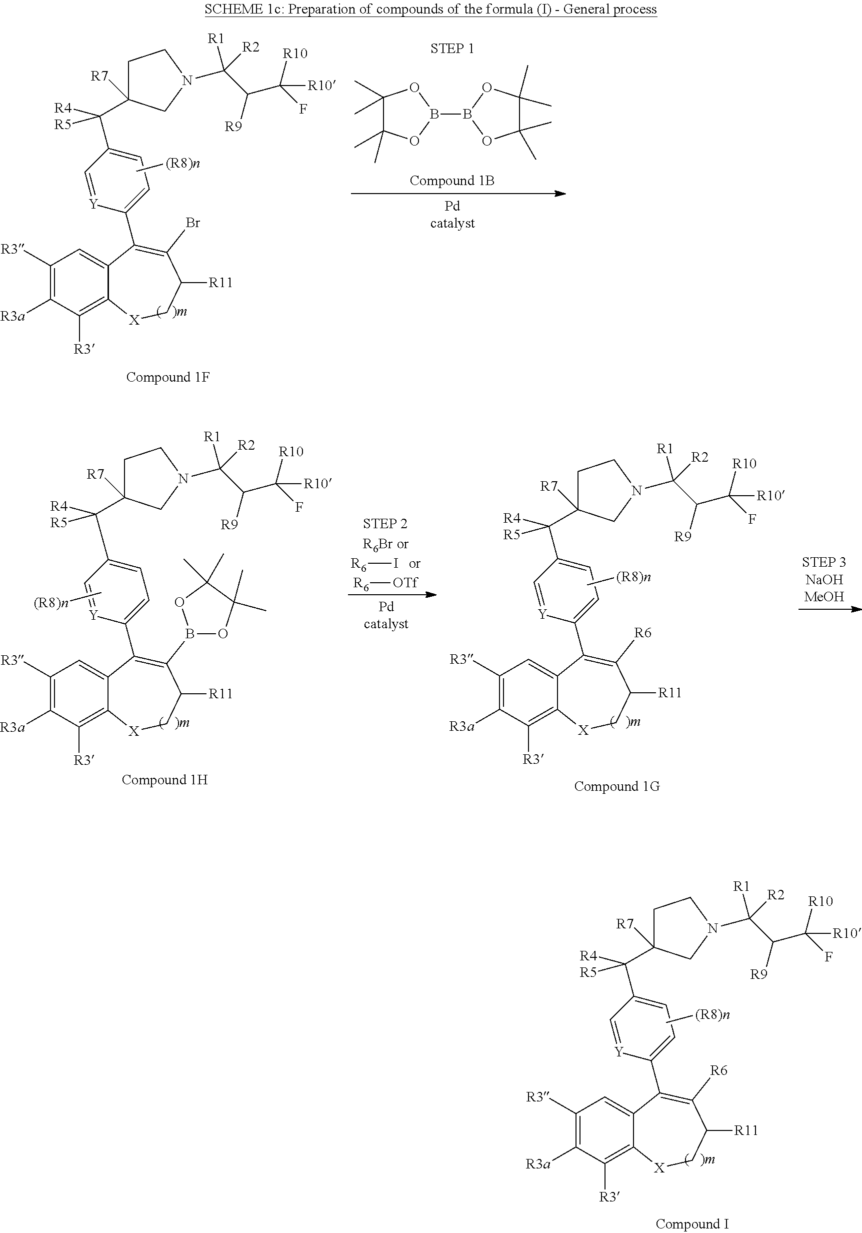

According to SCHEME 1c, in which R3a is a hydrogen atom, a carboxylic ester such as COOMe, COOEt, or protected OH such as 0-pivaloyl; R11 is a hydrogen atom and R1, R2, R3, R3′, R3″, R4, R5, R6, R7, R8, R9, R10, R10′, n, m, X and Y are as defined above, compound 1F can be converted in STEP 1 to compound 1H by treatment for example with compound 1B and with a palladium catalyst, for example bis(triphenylphosphine)palladium(II) dichloride Pd(PPh₃)₂Cl₂, and a phosphine such as triphenylphosphine in toluene by heating up to reflux of solvent in presence of a base such as KOPh.

Compound 1G wherein R6 is phenyl or heteroaryl can be prepared in a Suzuki coupling reaction between compounds 1H and either R6Br or R6I or R60Tf in STEP 2 using for example [1,1′-bis(diphenylphosphino) ferrocene]dichloropalladium(II) (Pd(dppf)Cl₂), complex with DCM as catalyst, in a mixture of dioxane and water and in the presence of a base, for example cesium carbonate (Cs₂CO₃), by heating up to reflux of solvent.

When R3a is COOMe, COOEt, or a protected OH such as O-pivaloyl, compound 1G can be converted in STEP 3 to compound of formula (I) in presence of a source of hydroxide ions such as NaOH in solution in methanol (MeOH). When R3 represents a —COOH group, extraction of the product can give the sodium salt of compound I. The acidification with an aqueous solution of HCl 2N to pH 6-7 can give the neutral form of compound I. The acidification with an aqueous solution of HCl 2N to pH 1-2 can give the hydrochloride salt of compound I. The purification using HPLC in presence of formic acid or trifluoroacetic acid in the eluent can give the formate or trifluoroacetate salt of compound I.

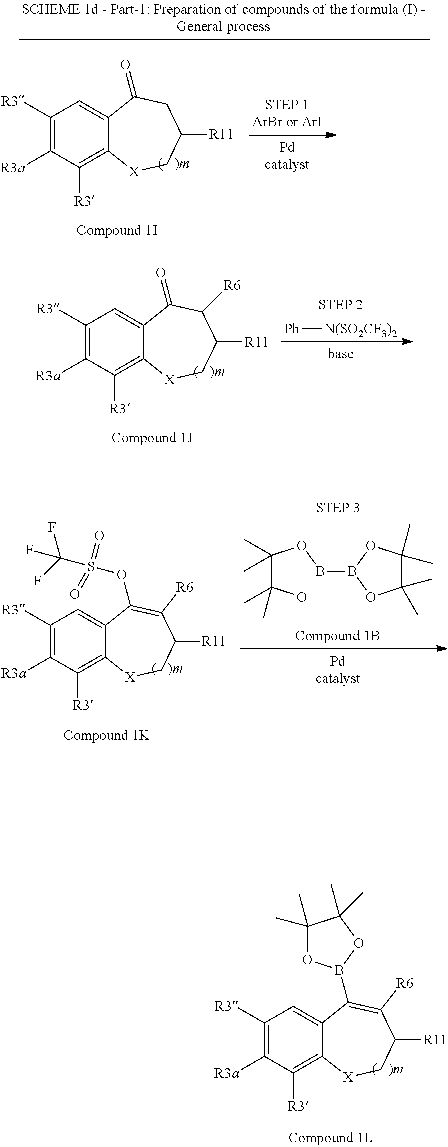

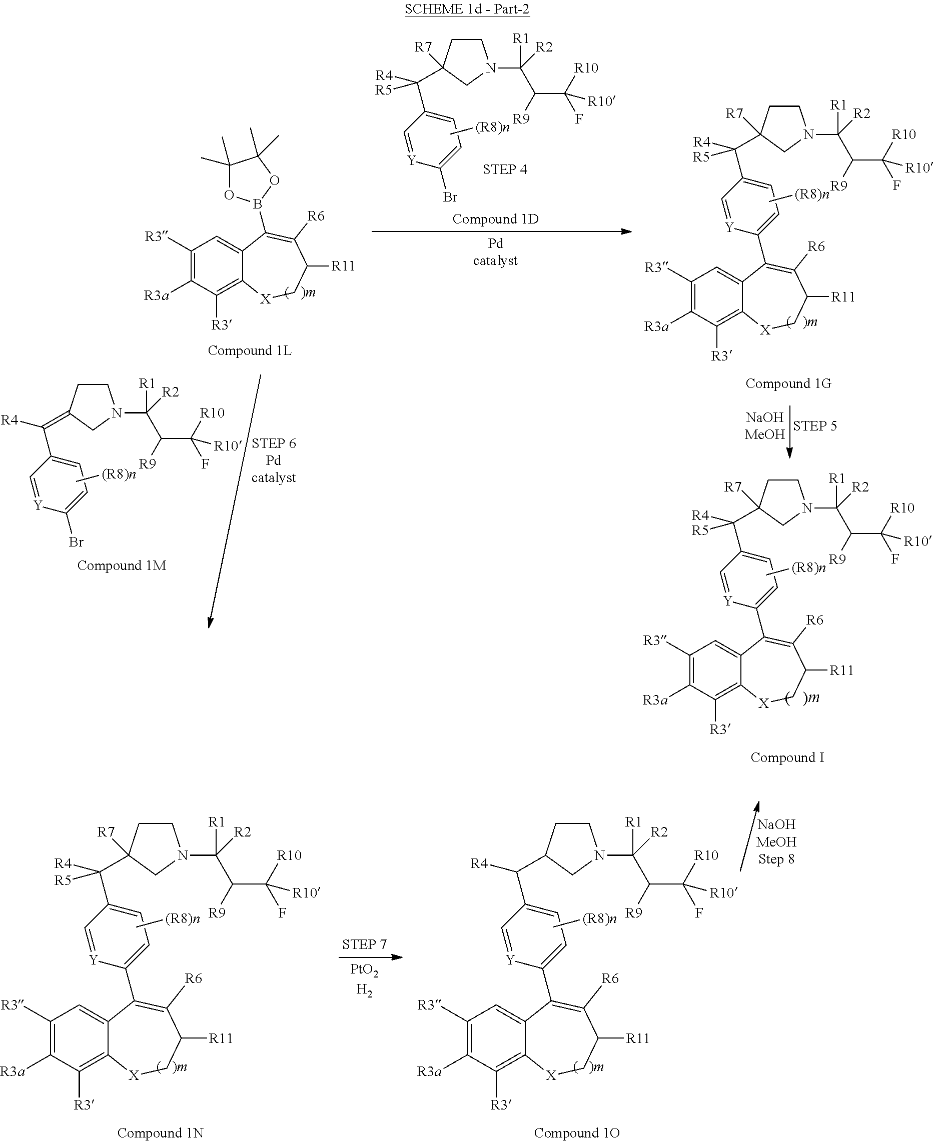

According to SCHEME 1d—Part-1 and Part-2, in which R3a is a hydrogen atom, a carboxylic ester such as COOMe, COOEt or protected OH such as O-pivaloyl, R11 is a hydrogen atom, R6 is a phenyl group or a heteroaryl group, R1, R2, R3, R3′, R3″, R4, R5, R7, R8, R9, R10, R10′, n, m, X and Y are as defined above, compound 1I can be converted in STEP 1 to compound 1J by treatment with aryl or heteroaryl bromide or iodide in the presence of a palladium catalyst, for example tris(dibenzylideneacetone)dipalladium(O) (Pd₂(dba)₃), in solution in toluene or xylene by heating up to reflux of solvent, in presence of a base such as K₂CO₃ or Cs₂CO₃. Alternative way to prepare compound 1J, wherein R6 can be any of the groups defined above for R6 in formula (I), is described in SCHEME 1g below.

Compound 1J can be converted in STEP 2 to compound 1K by treatment with N,N-bis(trifluoromethylsulfonyl)aniline in the presence of base such as DBU or NaH or KHMDS at −50° C. in a solvent such as MeTHF.

Compound 1K can be converted in STEP 3 to compound 1L by treatment for example with compound 1B, and with a palladium catalyst, for example bis (triphenylphosphine) palladium(II) dichloride Pd(PPh₃)₂Cl₂, and a phosphine such as triphenylphosphine in solution in toluene by heating up to reflux of solvent, in presence of a base such as KOPh.

Compound 1G can be prepared in a Suzuki coupling reaction between compounds 1L and 1D in STEP 4 using for example [1,1′-bis(diphenylphosphino)ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

Alternatively, compound 1L can be converted in STEP 6 to compound 1N in a Suzuki coupling reaction with compound 1M using for example [1,1′-bis(diphenylphosphino)ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

Compound 1N can be reduced to compound 10 in STEP 7 by hydrogenation with a catalyst, such as PtO₂ under hydrogen (H₂) pressure, around 2 bars for example, at room temperature.

When R3a is COOMe, COOEt, or a protected OH such as O-pivaloyl, compounds 1G or 10 can be converted in STEP 5 or STEP 8 to compound of formula (I) in presence of a source of hydroxide ions such as NaOH in solution in methanol (MeOH). When R3 represents a —COOH group, extraction of the product can give the sodium salt of compound I. The acidification with an aqueous solution of HCl 2N to pH 6-7 can give the neutral form of compound I. The acidification with an aqueous solution of HCl 2N to pH 1-2 can give the hydrochloride salt of compound I. The purification using HPLC in presence of formic acid or trifluoroacetic acid in the eluent can give the formate or trifluoroacetate salt of compound I.

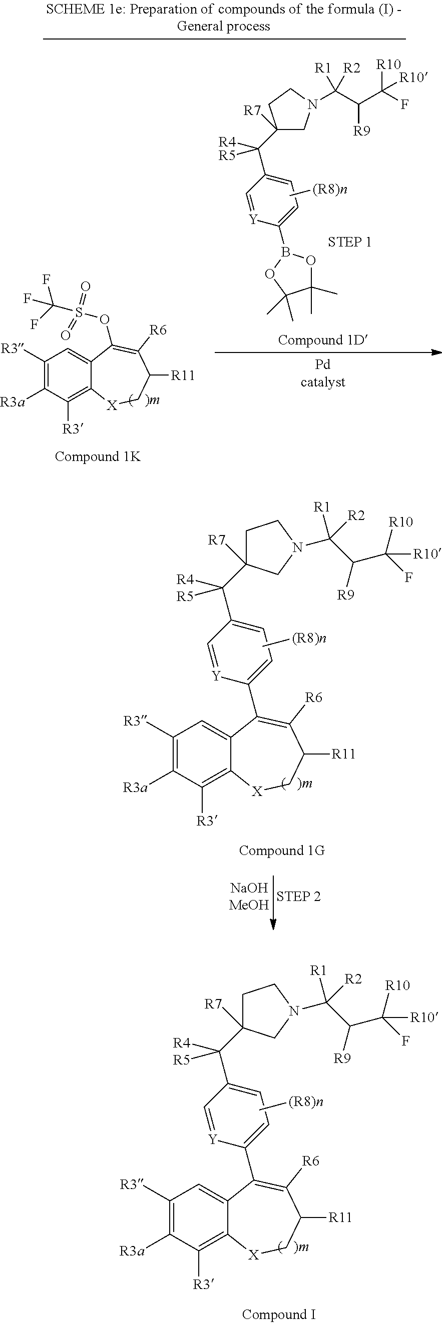

According to SCHEME 1e, in which R3a is a hydrogen atom, a carboxylic ester such as COOMe, COOEt or protected OH such as O-pivaloyl, and R1, R2, R3, R3′, R3″, R4, R5, R6, R7, R8, R9, R10, R10′, R11, n, m, X and Y are as defined above, compound 1K can be converted in STEP 1 to compound 1G by treatment with compound 1D′ in the presence of a palladium catalyst, for example [1,1′-bis(diphenylphosphino)ferrocene]dichloropalladium(I) (Pd(dppf)Cl₂), complex with DCM, in a mixture of dioxane and water and in the presence of a base, for example cesium carbonate (Cs₂CO₃), by heating up to reflux of solvent.

When R3a is COOMe, COOEt, or a protected OH such as O-pivaloyl, compound 1G can be converted in STEP 2 to compound of formula (I) in presence of a source of hydroxide ions such as NaOH in solution in methanol (MeOH). When R3 represents a —COOH group, extraction of the product can give the sodium salt of compound I. The acidification with an aqueous solution of HCl 2N to pH 6-7 can give the neutral form of compound 1. The acidification with an aqueous solution of HCl 2N to pH 1-2 can give the hydrochloride salt of compound I. The purification using HPLC in presence of formic acid or trifluoroacetic acid in the eluent can give the formate or trifluoroacetate salt of compound I.

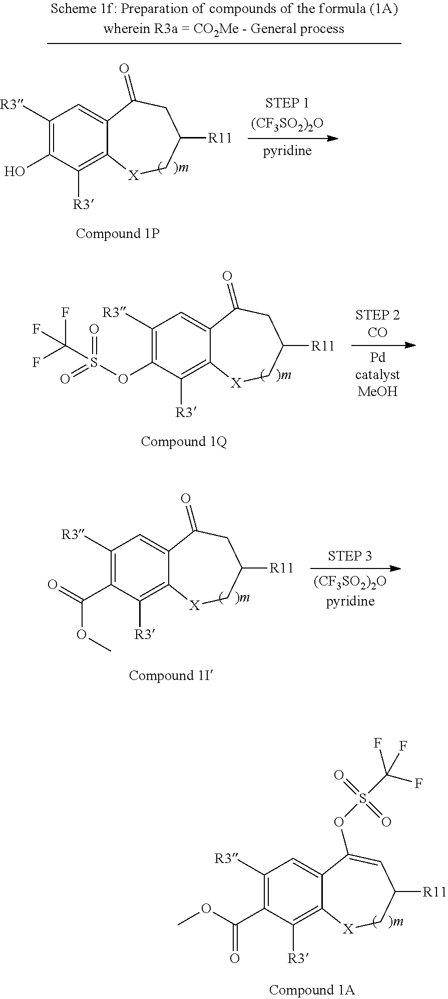

According to SCHEME 1f, in which X, m, R3′, R3″ and R11 are as defined above, compound 1A can be commercially available or prepared as follows: compound IP (commercially available or prepared according to WO2017140669 and WO2018091153) can be converted in STEP 1 to compound 1Q by treatment with trifluoromethanesulfonic anhydride, in solution in DCM, in the presence of pyridine as a base.

Compound 1Q can be converted in STEP 2 to compound 11′ by carbonylation with carbon monoxide, in solution in DMF and MeOH, in the presence of a palladium catalyst, for example [1,1′-bis(diphenylphosphino)ferrocene]dichloropalladium(II) (Pd(dppf)Cl₂), complex with DCM.

Compound 1I′ can be converted in STEP 3 to compound 1A wherein R3a=CO₂Me by treatment with trifluoromethanesulfonic anhydride, in solution in DCM, in the presence of pyridine as a base.

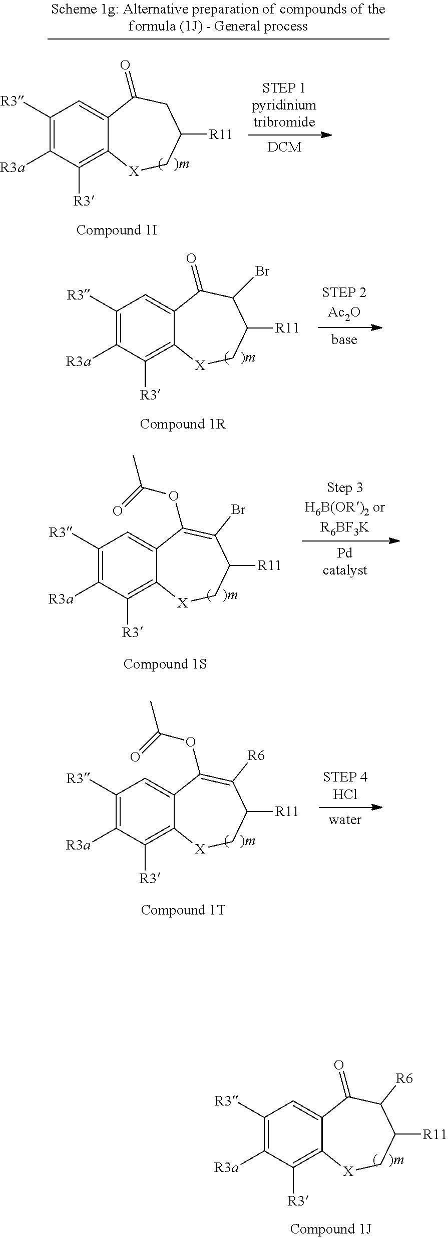

According to SCHEME 1g, in which R3a is a hydrogen atom, a carboxylic ester such as COOMe, COOEt, or protected OH with O-pivaloyl for example, R3′, R3″, R6, R11, X and m are as defined above, compound 1J can alternatively be prepared as follows: compound 1I can be converted in STEP 1 to compound 1R by treatment with pyridinium tribromide in DCM or THF at room temperature for example.

Compound 1R can be converted in STEP 2 to compound 1S by deprotonation with a base such as LiHMDS in THE followed by treatment with acetic anhydride.

Compound 1T can be prepared in STEP 3 in a Suzuki coupling reaction between compounds 1S and R₆B(OR′)₂ or R₆BF₃K wherein —B(OR′)₂ is a boronic acid or a pinacolate ester and R6 is as above defined, using for example [1,1′-bis(diphenylphosphino) ferrocene]dichloropalladium(II) (Pd(dppf)Cl₂), complex with DCM, as catalyst, in a mixture of toluene and water and in the presence of a base, for example cesium carbonate (Cs₂CO₃), by heating up to reflux of solvent.

Compound 1T can be converted in STEP 4 to compound 1J by hydrolysis with aqueous HCl solution by heating in methanol and DCM for example.

Herein is also provided a process for preparing a compound of formula (I) as defined above, wherein a compound of formula 1G

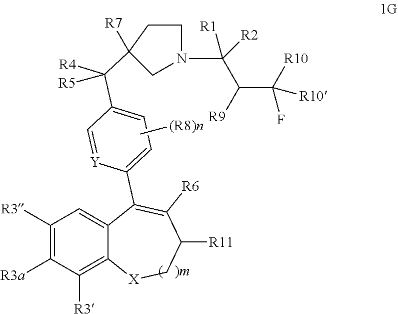

wherein R1, R2, R3′, R3″, R4, R5, R6, R7, R8, R9, R10, R10′, R11, n, m, X and Y are defined above and R3a is a hydrogen atom, a carboxylic ester such as COOMe, COOEt, or protected OH such as O-pivaloyl, is converted to compound of formula (I), in presence of a source of hydroxide ions, such as NaOH in solution in methanol, said step being optionally preceded by a step of obtaining compound 1G, wherein a compound of formula 1F

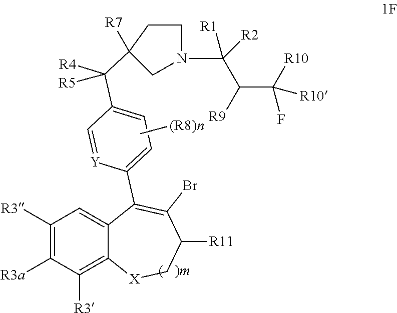

wherein R1, R2, R3′, R3″, R4, R5, R7, R8, R9, R10, R10′, R11, n, m, X and Y are as defined above and R3a is as defined above, is subjected to a Suzuki coupling with a boronic reagent R6-B(OR′)₂, wherein —B(OR′)₂ is a boronic acid or a pinacolate ester and R6 is as defined above.

Herein is also provided a process for preparing a compound of formula (I) as defined above, wherein a compound of formula 1Fa

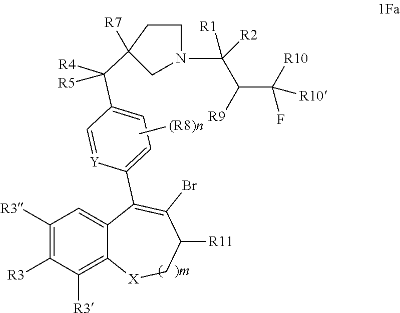

wherein R1, R2, R3, R3′, R3″, R4, R5, R7, R8, R9, R10, R10′, R11, n, m, X and Y are as defined above, is submitted to a Suzuki coupling a boronic reagent R6-B(OR′)₂, wherein —B(OR′)₂ is a boronic acid or a pinacolate ester and R6 is as defined above, said step being optionally preceded by a step for obtaining compound 1Fa, wherein a compound of formula 1F

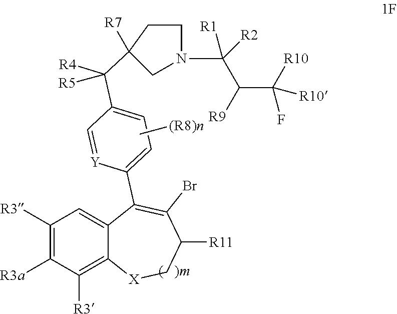

wherein R1, R2, R3′, R3″, R4, R5, R7, R8, R9, R10, R10′, R11, n, m, X and Y are as defined above and R3a is as defined above, is converted to a compound 1Fa in the presence of a source of hydroxide ions, such as NaOH in solution in methanol.

Herein are also provided the intermediate compounds selected from compounds of formula 1E, 1F, 1G and 1Fa, or any of its pharmaceutically acceptable salt,

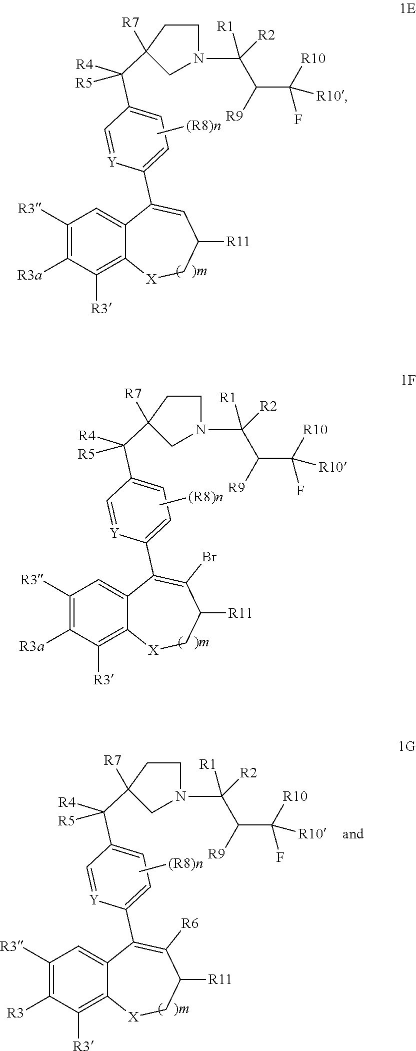

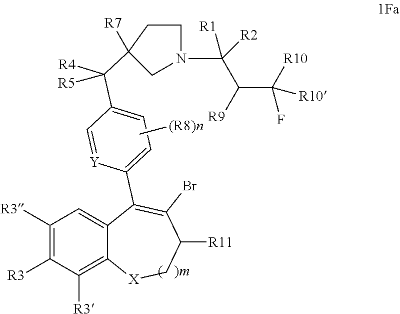

wherein R1, R2, R3, R3′, R3″, R4, R5, R6, R7, R8, R9, R10, R10′, R11, n, m, X and Y are as defined above and R3a is a hydrogen atom, a carboxylic ester such as COOMe, COOEt, or protected OH such as O-pivaloyl.

Herein are further provided the intermediate compounds of formula 1D and 1D′, or any of their pharmaceutically acceptable salt

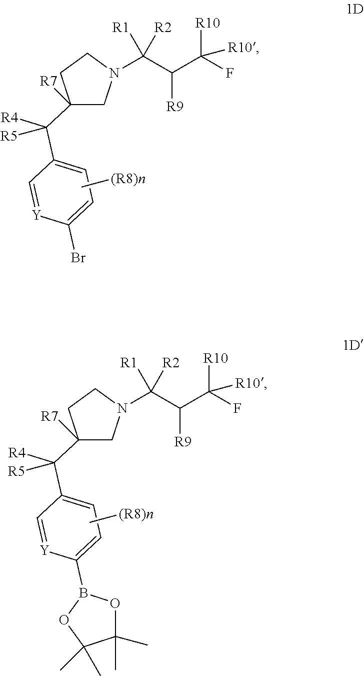

wherein R1, R2, R4, R5, R7, R8, R9, R10, R10′, n and Y are as defined above.

In another aspect, herein is also provided a process for the preparation of a compound of formula (I), wherein R3 is a —COOH group, comprising a deprotection step of a compound of formula 1G as defined above, optionally followed by a purification step.

Said purification step may for example consist, as illustrated in step 2 of example 1 hereinafter, in an acidification step, for example with an aqueous solution of hydrochloric acid.

Herein is further provided the intermediate compound of formula 1L, or any of its pharmaceutically acceptable salt

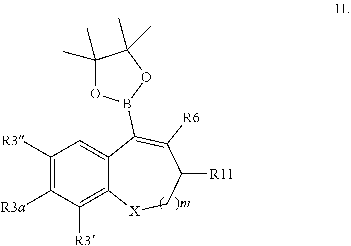

wherein R3a, R3′, R3″, X, m and R6 are as defined above and R11 is a hydrogen atom.

The ¹H NMR Spectra at 400 and 500 MHz were performed on a Bruker Avance DRX-400 and Bruker Avance DPX-500 spectrometer, respectively, with the chemical shifts (S in ppm) in the solvent dimethyl sulfoxide-d6 (d6-DMSO) referenced at 2.5 ppm at a temperature of 303 K. Coupling constants (J) are given in Hertz.

The liquid chromatography/mass spectra (LC/MS) were obtained on a UPLC Acquity Waters instrument, light scattering detector Sedere and SQD Waters mass spectrometer using UV detection DAD 210-400 nm and flash Acquity UPLC CSH C18 1.7 μm, dimension 2.1×30 mm, mobile phase H₂O+0.1% HCO₂H/CH₃CN+0.1% HCO₂H.

The following tables 1a and 1b comprise respectively specific compounds of formula (I) (name and structure) in accordance with the present disclosure as well their characterization (¹H NMR and liquid chromatography/mass).

TABLE 1a

| Example or | | |
|---|---|---|
| compound | Structure | Name |
| 1 | 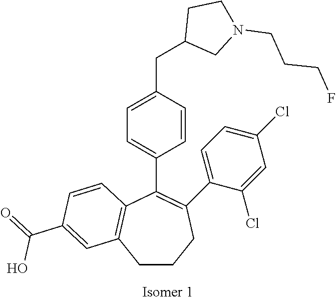 [SMILES: O=C(O)c1ccc2c(c1)CCCC(c1ccc(Cl)cc1Cl)=C2c1ccc(CC2CCN(CCCF)C2)cc1] | 8-(2,4-dichlorophenyl)-9-(4-((1-(3- fluoropropyl)pyrrolidin-3- yl)methyl)phenyl)-6,7-dihydro-5H- benzo[7]annulene-3-carboxylic acid, (S) Isomer |
| 2 | 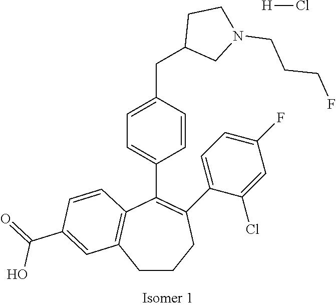 | 8-(2-chloro-4-fluorophenyl)-9-(4-((1-(3- fluoropropyl)pyrrolidin-3- yl)methyl)phenyl)-6,7-dihydro-5H- benzo[7]annulene-3-carboxylic acid hydrochloride, Isomer 1 |
| 3 | 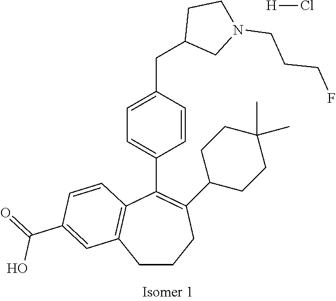 | 8-(4,4-dimethylcyclohexyl)-9-(4-((1-(3- fluoropropyl)pyrrolidin-3- yl)methyl)phenyl)-6,7-dihydro-5H- benzo[7]annulene-3-carboxylic acid, Isomer 1 |
| 4 | 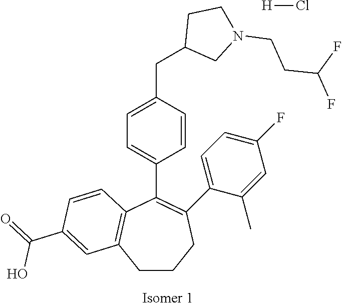 | 9-(4-((1-(3,3-difluoropropyl)pyrrolidin-3- yl)methyl)phenyl)-8-(4-fluoro-2- methylphenyl)-6,7-dihydro-5H- benzo[7]annulene-3-carboxylic acid hydrochloride, Isomer 1 |
| 5 | 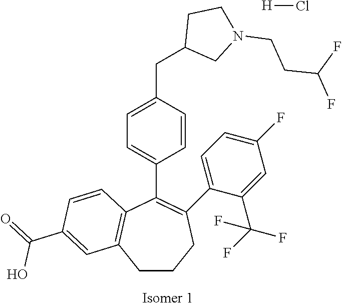 | 9-(4-((1-(3,3-difluoropropyl)pyrrolidin-3- yl)methyl)phenyl)-8-(4-fluoro-2- (trifluoromethyl)phenyl)-6,7-dihydro-5H- benzo[7]annulene-3-carboxylic acid hydrochloride, Isomer 1 |
| 6 | 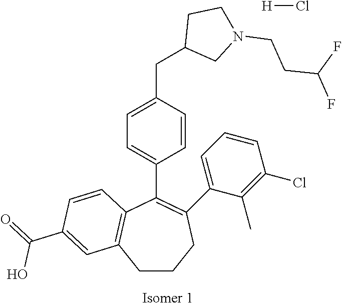 | 8-(3-chloro-2-methylphenyl)-9-(4-((1- (3,3-difluoropropyl)pyrrolidin-3- yl)methyl)phenyl)-6,7-dihydro-5H- benzo[7]annulene-3-carboxylic acid hydrochloride, Isomer 1 |
| 7 | 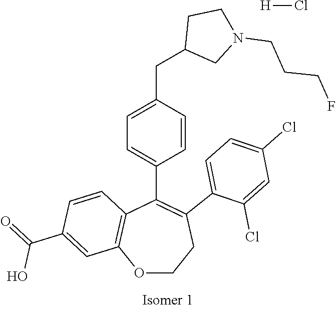 | 4-(2,4-dichlorophenyl)-5-(4-((1-(3- fluoropropyl)pyrrolidin-3- yl)methyl)phenyl)-2,3- dihydrobenzo[b]oxepine-8-carboxylic acid hydrochloride, Isomer 1 |
| 8 | 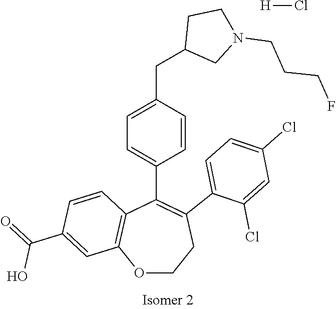 | 4-(2,4-dichlorophenyl)-5-(4-((1-(3- fluoropropyl)pyrrolidin-3- yl)methyl)phenyl)-2,3- dihydrobenzo[b]oxepine-8-carboxylic acid hydrochloride, Isomer 2 |
| 9 | 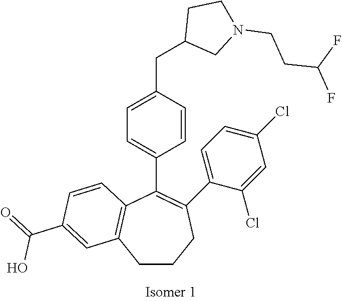 [SMILES: O=C(O)c1ccc2c(c1)CCCC(c1ccc(Cl)cc1Cl)=C2c1ccc(CC2CCN(CCC(F)F)C2)cc1] | 8-(2,4-dichlorophenyl)-9-(4-((1-(3,3- difluoropropyl)pyrrolidin-3- yl)methyl)phenyl)-6,7-dihydro-5H- benzo[7]annulene-3-carboxylic acid, Isomer 1 |
| 10 | 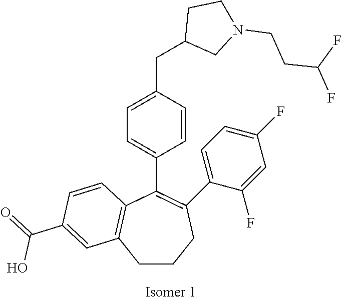 [SMILES: O=C(O)c1ccc2c(c1)CCCC(c1ccc(F)cc1F)=C2c1ccc(CC2CCN(CCC(F)F)C2)cc1] | 8-(2,4-difluorophenyl)-9-(4-((1-(3,3- difluoropropyl)pyrrolidin-3- yl)methyl)phenyl)-6,7-dihydro-5H- benzo[7]annulene-3-carboxylic acid, Isomer 1 |
| 11 | 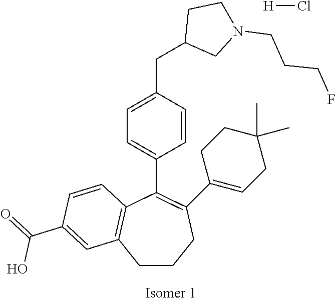 | 8-(4,4-dimethylcyclohex-1-en-1-yl)-9-(4- ((1-(3-fluoropropyl)pyrrolidin-3- yl)methyl)phenyl)-6,7-dihydro-5H- benzo[7]annulene-3-carboxylic acid hydrochloride, Isomer 1 |
| 12 | 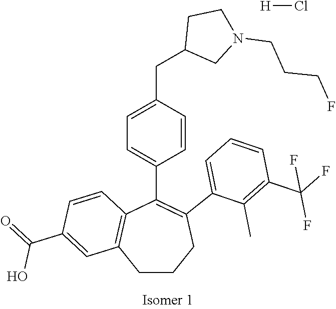 | 9-(4-((1-(3-fluoropropyl)pyrrolidin-3- yl)methyl)phenyl)-8-(2-methyl-3- (trifluoromethyl)phenyl)-6,7-dihydro-5H- benzo[7]annulene-3-carboxylic acid hydrochloride, Isomer 1 |
| 13 | 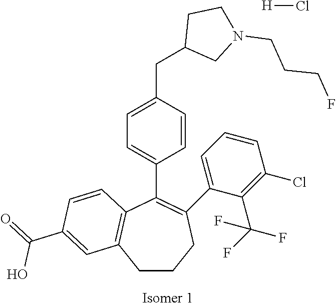 | 8-(3-chloro-2-(trifluoromethyl)phenyl)-9- (4-((1-(3-fluoropropyl)pyrrolidin-3- yl)methyl)phenyl)-6,7-dihydro-5H- benzo[7]annulene-3-carboxylic acid hydrochloride, Isomer 1 |
| 14 | 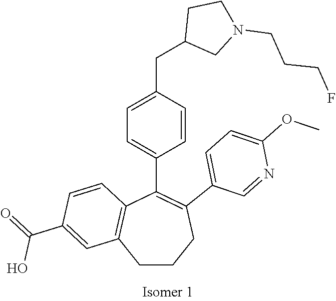 [SMILES: COc1ccc(C2=C(c3ccc(CC4CCN(CCCF)C4)cc3)c3ccc(C(=O)O)cc3CCC2)cn1] | 9-(4-((1-(3-fluoropropyl)pyrrolidin-3- yl)methyl)phenyl)-8-(6-methoxypyridin- 3-yl)-6,7-dihydro-5H-benzo[7]annulene- 3-carboxylic acid, Isomer 1 |
| 15 | 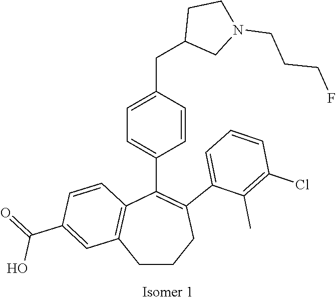 [SMILES: Cc1c(Cl)cccc1C1=C(c2ccc(CC3CCN(CCCF)C3)cc2)c2ccc(C(=O)O)cc2CCC1] | 8-(3-chloro-2-methylphenyl)-9-(4-((1-(3- fluoropropyl)pyrrolidin-3- yl)methyl)phenyl)-6,7-dihydro-5H- benzo[7]annulene-3-carboxylic acid, Isomer 1 |
| 16 | 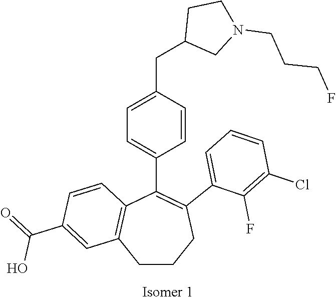 [SMILES: O=C(O)c1ccc2c(c1)CCCC(c1cccc(Cl)c1F)=C2c1ccc(CC2CCN(CCCF)C2)cc1] | 8-(3-chloro-2-fluorophenyl)-9-(4-((1-(3- fluoropropyl)pyrrolidin-3- yl)methyl)phenyl)-6,7-dihydro-5H- benzo[7]annulene-3-carboxylic acid, Isomer 1 |
| 17 | 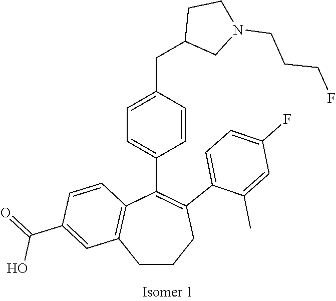 [SMILES: Cc1cc(F)ccc1C1=C(c2ccc(CC3CCN(CCCF)C3)cc2)c2ccc(C(=O)O)cc2CCC1] | 8-(4-fluoro-2-methylphenyl)-9-(4-((1-(3- fluoropropyl)pyrrolidin-3- yl)methyl)phenyl)-6,7-dihydro-5H- benzo[7]annulene-3-carboxylic acid, Isomer 1 |
| 18 | 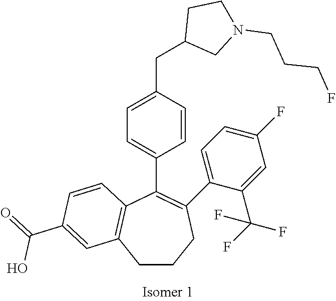 [SMILES: O=C(O)c1ccc2c(c1)CCCC(c1ccc(F)cc1C(F)(F)F)=C2c1ccc(CC2CCN(CCCF)C2)cc1] | 8-(4-fluoro-2-(trifluoromethyl)phenyl)-9- (4-((1-(3-fluoropropyl)pyrrolidin-3- yl)methyl)phenyl)-6,7-dihydro-5H- benzo[7]annulene-3-carboxylic acid, Isomer 1 |
| 19 | 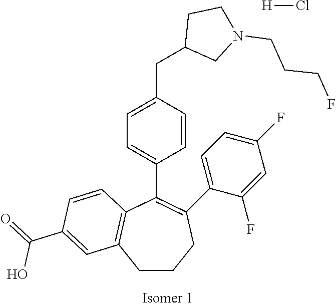 | 8-(2,4-difluorophenyl)-9-(4-((1-(3- fluoropropyl)pyrrolidin-3- yl)methyl)phenyl)-6,7-dihydro-5H- benzo[7]annulene-3-carboxylic acid hydrochloride, Isomer 1 |
| 20 | 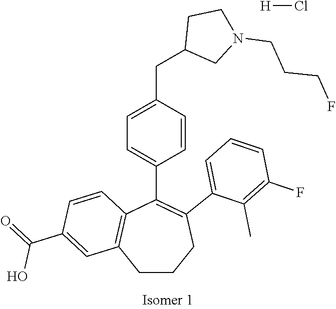 | 8-(3-fluoro-2-methylphenyl)-9-(4-((1-(3- fluoropropyl)pyrrolidin-3- yl)methyl)phenyl)-6,7-dihydro-5H- benzo[7]annulene-3-carboxylic acid hydrochloride, Isomer 1 |
| 21 | 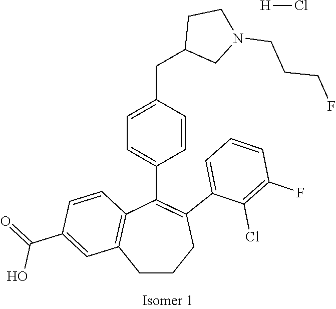 | 8-(2-chloro-3-fluorophenyl)-9-(4-((1-(3- fluoropropyl)pyrrolidin-3- yl)methyl)phenyl)-6,7-dihydro-5H- benzo[7]annulene-3-carboxylic acid hydrochloride, Isomer 1 |
| 22 | 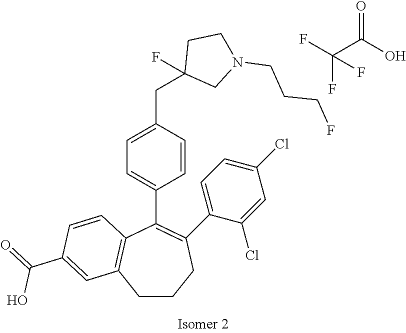 | 8-(2,4-dichlorophenyl)-9-(4-((3-fluoro-1- (3-fluoropropyl)pyrrolidin-3- yl)methyl)phenyl)-6,7-dihydro-5H- benzo[7]annulene-3-carboxylic acid, 2,2,2-trifluoroacetic acid, Isomer 2 |
| 23 | 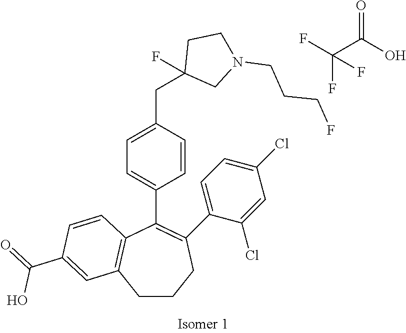 | 8-(2,4-dichlorophenyl)-9-(4-((3-fluoro-1- (3-fluoropropyl)pyrrolidin-3- yl)methyl)phenyl)-6,7-dihydro-5H- benzo[7]annulene-3-carboxylic acid, 2,2,2-trifluoroacetic acid, Isomer 1 |
| 24 | 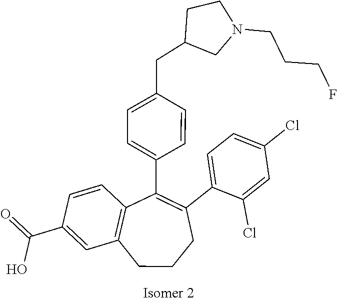 [SMILES: O=C(O)c1ccc2c(c1)CCCC(c1ccc(Cl)cc1Cl)=C2c1ccc(CC2CCN(CCCF)C2)cc1] | 8-(2,4-dichlorophenyl)-9-(4-((1-(3- fluoropropyl)pyrrolidin-3- yl)methyl)phenyl)-6,7-dihydro-5H- benzo[7]annulene-3-carboxylic acid, (R) Isomer |
| 25 | 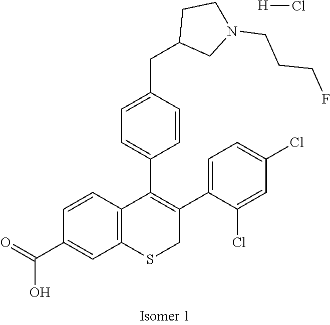 | 3-(2,4-dichlorophenyl)-4-(4-((1-(3- fluoropropyl)pyrrolidin-3- yl)methyl)phenyl)-2H-thiochromene-7- carboxylic acid hydrochloride, Isomer 1 |
| 26 | 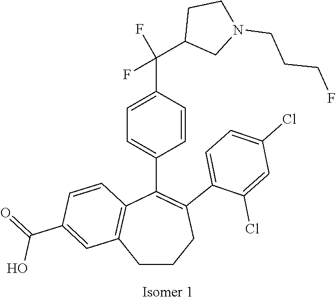 [SMILES: O=C(O)c1ccc2c(c1)CCCC(c1ccc(Cl)cc1Cl)=C2c1ccc(C(F)(F)C2CCN(CCCF)C2)cc1] | 8-(2,4-dichlorophenyl)-9-(4-(difluoro(1- (3-fluoropropyl)pyrrolidin-3- yl)methyl)phenyl)-6,7-dihydro-5H- benzo[7]annulene-3-carboxylic acid, Isomer 1 |
| 27 | 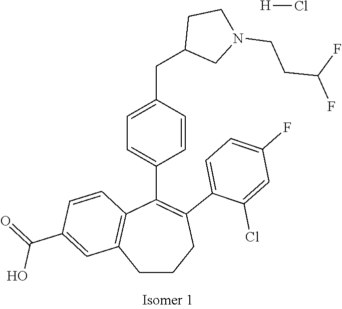 | 8-(2-chloro-4-fluorophenyl)-9-(4-((1- (3,3-difluoropropyl)pyrrolidin-3- yl)methyl)phenyl)-6,7-dihydro-5H- benzo[7]annulene-3-carboxylic acid hydrochloride, Isomer 1 |
| 28 | 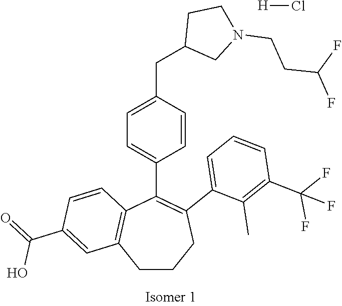 | 9-(4-((1-(3,3-difluoropropyl)pyrrolidin-3- yl)methyl)phenyl)-8-(2-methyl-3- (trifluoromethyl)phenyl)-6,7-dihydro-5H- benzo[7]annulene-3-carboxylic acid hydrochloride, Isomer 1 |
| 29 | 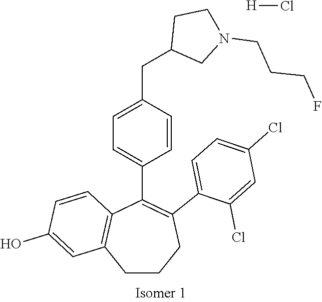 | 8-(2,4-dichlorophenyl)-9-(4-((1-(3- fluoropropyl)pyrrolidin-3- yl)methyl)phenyl)-6,7-dihydro-5H- benzo[7]annulene-3-ol hydrochloride, Isomer 1 |
| 30 | 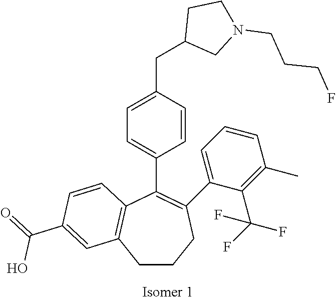 [SMILES: Cc1cccc(C2=C(c3ccc(CC4CCN(CCCF)C4)cc3)c3ccc(C(=O)O)cc3CCC2)c1C(F)(F)F] | 9-(4-((1-(3-fluoropropyl)pyrrolidin-3- yl)methyl)phenyl)-8-(3-methyl-2- (trifluoromethyl)phenyl)-6, 7-dihydro-5H- benzo[7]annulene-3-carboxylic acid, Isomer 1 |
| 31 | 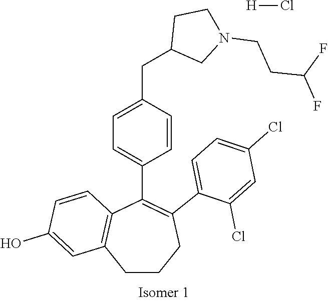 | 8-(2,4-dichlorophenyl)-9-(4-((1-(3,3- difluoropropyl)pyrrolidin-3- yl)methyl)phenyl)-6,7-dihydro-5H- benzo[7]annulen-3-ol hydrochloride, Isomer 1 |
| 32 | 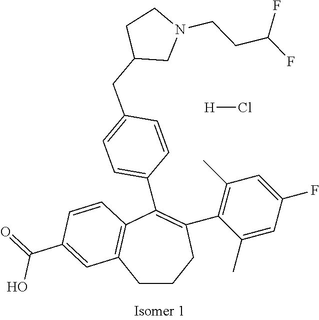 [SMILES: Cc1cc(F)cc(C)c1C1=C(c2ccc(CC3CCN(CCC(F)F)C3)cc2)c2ccc(C(=O)O)cc2CCC1 ; Cl] | 9-(4-((1-(3,3-difluoropropyl)pyrrolidin-3- yl)methyl)phenyl)-8-(4-fluoro-2,6- dimethylphenyl)-6,7-dihydro-5H- benzo[7]annulene-3-carboxylic acid hydrochloride, Isomer 1 |
| 33 | 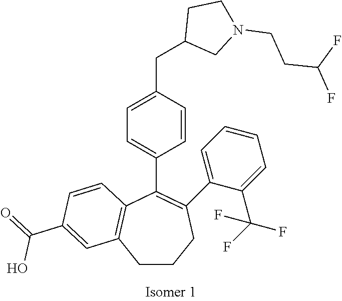 [SMILES: O=C(O)c1ccc2c(c1)CCCC(c1ccccc1C(F)(F)F)=C2c1ccc(CC2CCN(CCC(F)F)C2)cc1] | 9-(4-((1-(3,3-difluoropropyl)pyrrolidin-3- yl)methyl)phenyl)-8-(2- (trifluoromethyl)phenyl)-6,7-dihydro-5H- benzo[7]annulene-3-carboxylic acid |
| 34 | 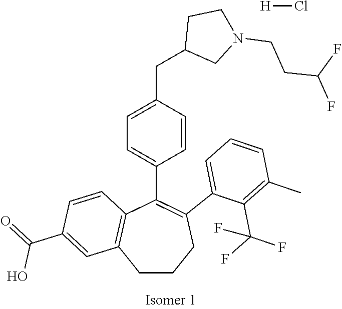 | 9-(4-((1-(3,3-difluoropropyl)pyrrolidin-3- yl)methyl)phenyl)-8-(3-methyl-2- (trifluoromethyl)phenyl)-6,7-dihydro-5H- benzo[7]annulene-3-carboxylic acid hydrochloride, Isomer 1 |
| 35 | 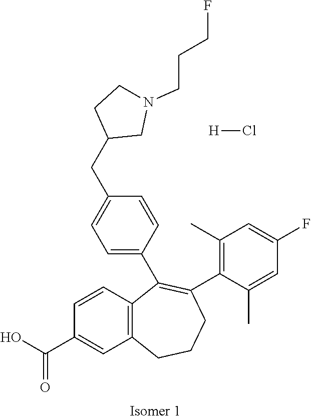 [SMILES: Cc1cc(F)cc(C)c1C1=C(c2ccc(CC3CCN(CCCF)C3)cc2)c2ccc(C(=O)O)cc2CCC1 ; Cl] | 8-(4-fluoro-2,6-dimethylphenyl)-9-(4- ((1-(3-fluoropropyl)pyrrolidin-3- yl)methyl)phenyl)-6,7-dihydro-5H- benzo[7]annulene-3-carboxylic acid hydrochloride, Isomer 1 |
| 36 | 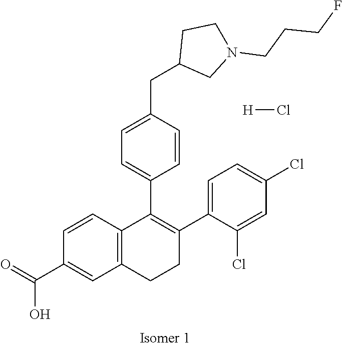 [SMILES: O=C(O)c1ccc2c(c1)CCC(c1ccc(Cl)cc1Cl)=C2c1ccc(CC2CCN(CCCF)C2)cc1 ; Cl] | 6-(2,4-dichlorophenyl)-5-(4-((1-(3- fluoropropyl)pyrrolidin-3- yl)methyl)phenyl)-7,8- dihydronaphthalene-2-carboxylic acid hydrochloride, Isomer 1 |
| 37 | 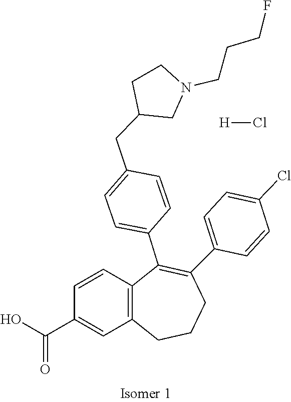 | 8-(4-chlorophenyl)-9-(4-((1-(3- fluoropropyl)pyrrolidin-3- yl)methyl)phenyl)-6,7-dihydro-5H- benzo[7]annulene-3-carboxylic acid hydrochloride, Isomer 1 |
| 38 | 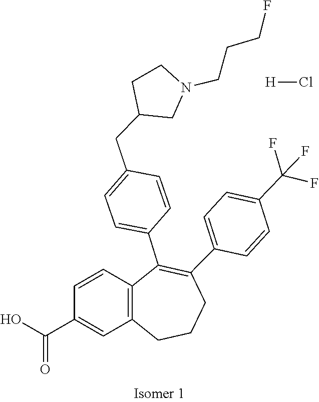 | 9-(4-((1-(3-fluoropropyl)pyrrolidin-3- yl)methyl)phenyl)-8-(4- (trifluoromethyl)phenyl)-6,7-dihydro-5H- benzo[7]annulene-3-carboxylic acid hydrochloride, Isomer 1 |
| 39 | 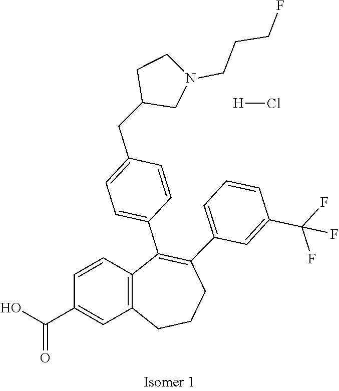 | 9-(4-((1-(3-fluoropropyl)pyrrolidin-3- yl)methyl)phenyl)-8-(3- (trifluoromethyl)phenyl)-6,7-dihydro-5H- benzo[7]annulene-3-carboxylic acid hydrochloride, Isomer 1 |
| 40 | 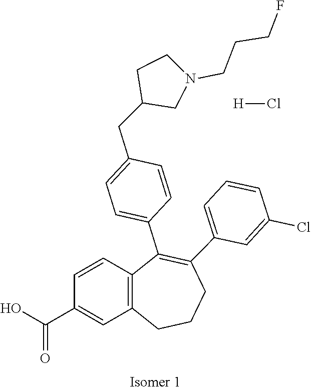 | 8-(3-chlorophenyl)-9-(4-((1-(3- fluoropropyl)pyrrolidin-3- yl)methyl)phenyl)-6,7-dihydro-5H- benzo[7]annulene-3-carboxylic acid hydrochloride, Isomer 1 |
| 41 | 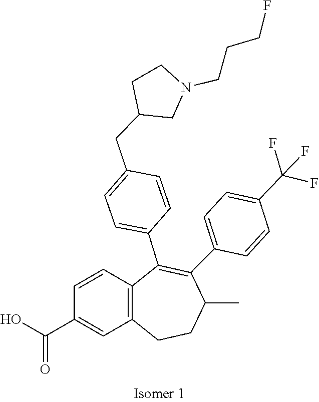 [SMILES: CC1CCc2cc(C(=O)O)ccc2C(c2ccc(CC3CCN(CCCF)C3)cc2)=C1c1ccc(C(F)(F)F)cc1] | 9-(4-((1-(3-fluoropropyl)pyrrolidin-3- yl)methyl)phenyl)-7-methyl-8-(4- (trifluoromethyl)phenyl)-6,7-dihydro-5H- benzo[7]annulene-3-carboxylic acid, Isomer 1 |
| 42 | 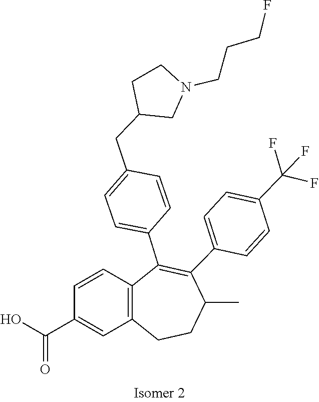 [SMILES: CC1CCc2cc(C(=O)O)ccc2C(c2ccc(CC3CCN(CCCF)C3)cc2)=C1c1ccc(C(F)(F)F)cc1] | 9-(4-((1-(3-fluoropropyl)pyrrolidin-3- yl)methyl)phenyl)-7-methyl-8-(4- (trifluoromethyl)phenyl)-6,7-dihydro-5H- benzo[7]annulene-3-carboxylic acid, Isomer 2 |
| 43 | 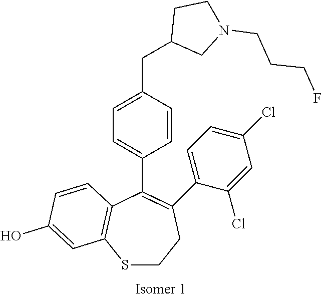 [SMILES: Oc1ccc2c(c1)SCCC(c1ccc(Cl)cc1Cl)=C2c1ccc(CC2CCN(CCCF)C2)cc1] | 4-(2,4-dichlorophenyl)-5-(4-((1-(3- fluoropropyl)pyrrolidin-3- yl)methyl)phenyl)-2,3- dihydrobenzo[b]thiepin-8-ol, Isomer 1 |
| 44 | 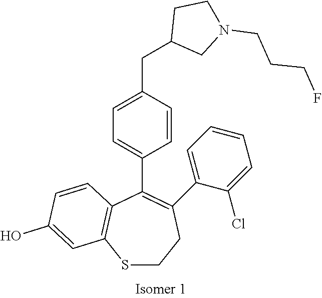 [SMILES: Oc1ccc2c(c1)SCCC(c1ccccc1Cl)=C2c1ccc(CC2CCN(CCCF)C2)cc1] | 4-(2-chlorophenyl)-5-(4-((1-(3- fluoropropyl)pyrrolidin-3- yl)methyl)phenyl)-2,3- dihydrobenzo[b]thiepin-8-ol, Isomer 1 |

TABLE 1b

| | | | MASS: |
|---|---|---|---|
| Example | | | LC/MS |
| or | Preparation | | (m/z, |
| compound | Method | NMR | MH+): |
| 1 | A | 1H NMR (400 MHz, DMSO-d6) δ ppm 1.28- | 552 |
| | | 1.38 (m, 1 H), 1.62-1.86 (m, 3 H), 1.98-2.08 | |
| | | (m, 1 H), 2.10-2.32 (m, 6 H), 2.35-2.46 (m, 6 | |
| | | H), 2.86-2.99 (m, 2 H), 4.45 (dt, J = 47, 6 Hz, 2 | |
| | | H), 6.76 (d, J = 8 Hz, 2 H), 6.85 (d, J = 8 Hz, 1 H), | |
| | | 6.96 (d, J = 8 Hz, 2 H), 7.16 (m, 1 H), 7.24 (m, 1 | |
| | | H), 7.56 (d, J = 2 Hz, 1 H), 7.74 (dd, J = 8, 2 Hz, 1 | |
| | | H), 7.90 (d, J = 1 Hz, 1 H) | |
| 2 | B | 1H NMR (400 MHz, DMSO-d6) δ ppm 1.33 (m, 1 | 536 |
| | | H), 1.65-1.85 (m, 3 H), 2.06 (dt, J = 9, 6 Hz, 1 H), | |
| | | 2.11-2.48 (m, 12 H), 2.63 (m, 1 H), 2.89-2.98 | |
| | | (m, 2 H), 4.45 (dt, J = 48, 6 Hz, 2 H), 6.75 (d, J = 8 | |
| | | Hz, 2 H), 6.87 (d, J = 8 Hz, 1 H), 6.96 (d, J = 8 Hz, 2 | |
| | | H), 7.05 (br t, J = 8 Hz, 1 H), 7.21 (m, 1 H), 7.41 | |
| | | (dd, J = 9, 3 Hz, 1 H), 7.75 (dd, J = 8, 2 Hz, 1 H), | |
| | | 7.91 (d, J = 2 Hz, 1 H), 10.15-13.22 (m, 1 H) | |
| 3 | D | 1H NMR (400 MHz, DMSO-d6) δ ppm 0.83 (s, 3 | 518 |
| | | H), 0.91 (s, 4 H), 0.99 (t, J = 12 Hz, 2 H), 1.38 (t, | |
| | | J = 14 Hz, 4 H), 1.51-1.68 (m, 2 H), 1.82-2.05 | |
| | | (m, 5 H), 2.07-2.20 (m, 2 H), 2.21-2.31 (m, 2 | |
| | | H), 2.48-3.28 (m, 10 H), 4.50 (dt, J = 47, 6 Hz, 2 | |
| | | H), 6.74 (d, J = 8 Hz, 1 H), 6.98 (d, J = 8 Hz, 2 H), | |
| | | 7.19 (d, J = 8 Hz, 2 H), 7.65 (dd, J = 8, 2 Hz, 1 H), | |
| | | 7.82 (d, J = 2 Hz, 1 H), 9.93-10.82 (m, 1 H), | |
| | | 12.25-13.03 (m, 1 H) | |
| 4 | A | 1H NMR (400 MHz, DMSO-d6) δ ppm 1.42- | 534 |
| | | 2.04 (m, 4 H), 2.08-2.20 (m, 6 H), 2.20-2.44 | |
| | | (m, 6 H), 2.54-2.66 (m, 2 H), 2.79-3.02 (m, 2 | |
| | | H), 3.18-3.26 (m, 2 H), 6.21 (br t, J = 56 Hz, 1 H), | |
| | | 6.73 (d, J = 8 Hz, 2 H), 6.83-6.91 (m, 2 H), 6.93- | |
| | | 7.08 (m, 4 H), 7.75 (dd, J = 8, 2 Hz, 1 H), 7.92 (d, | |
| | | J = 2 Hz, 1 H), 9.74-11.04 (m, 1 H), 12.45-13.13 | |
| | | (m, 1 H) | |
| 5 | B | 1H NMR (400 MHz, DMSO-d6) δ ppm 1.34- | 588 |
| | | 1.60 (m, 1 H), 1.78-1.95 (m, 1 H), 2.02-2.26 | |
| | | (m, 7 H), 2.53-2.64 (m, 2 H), 2.78-3.04 (m, 4 | |
| | | H), 3.16-3.22 (m, 4 H), 6.15 (tt, J = 57, 5 Hz, 1 | |
| | | H), 6.76 (d, J = 8 Hz, 2 H), 6.87 (d, J = 8 Hz, 1 H), | |
| | | 6.98 (d, J = 8 Hz, 2 H), 7.18-7.29 (m, 1 H), 7.30- | |
| | | 7.41 (m, 1 H), 7.62 (dd, J = 9, 3 Hz, 1 H), 7.75 (dd, | |
| | | J = 8, 2 Hz, 1 H), 7.92 (d, J = 2 Hz, 1 H), 8.72-9.89 | |
| | | (m, 1 H), 9.69-11.46 (m, 1 H) | |
| 6 | B | 1H NMR (400 MHz, DMSO-d6) δ ppm 1.40- | 550 |
| | | 2.05 (m, 4 H), 2.10-2.19 (m, 5 H), 2.22 (s, 3 H), | |
| | | 2.25-2.65 (m, 6 H), 2.80-3.22 (m, 4 H), 6.21 | |
| | | (tt, J = 56, 4 Hz, 1 H), 6.73 (d, J = 7 Hz, 2 H), 6.88 | |
| | | (d, J = 8 Hz, 1 H), 6.92-7.02 (m, 3 H), 7.05 (t, J = 8 | |
| | | Hz, 1 H), 7.25 (d, J = 8 Hz, 1 H), 7.76 (dd, J = 8, 2 | |
| | | Hz, 1 H), 7.93 (d, J = 2 Hz, 1 H), 9.82-10.82 (m, 1 | |
| | | H), 12.46-13.25 (m, 1 H) | |
| 7 | A | 1H NMR (400 MHz, DMSO-d6) δ ppm 1.46- | 554 |
| | | 1.72 (m, 1 H), 1.79-2.14 (m, 3 H), 2.52-3.32 (m | |
| | | partially hidden, 10 H), 4.37-4.70 (m, 4 H), 6.85 | |
| | | (dd, J = 8, 2 Hz, 3 H), 6.98-7.12 (m, 3 H), 7.24 | |
| | | (dd, J = 8, 2 Hz, 2 H), 7.52-7.67 (m, 3 H), 10.11 | |
| | | (s, 1 H), 12.67-13.46 (br s, 1 H) | |
| 8 | A | 1H NMR (400 MHz, DMSO-d6) δ ppm 1.37 (m, 1 | 554 |
| | | H), 1.70-1.90 (m, 2 H), 2.13 (m, 1 H), 2.24-2.79 | |
| | | (m partially hidden, 11 H), 4.46 (dt, J = 47, 6 Hz, 2 | |
| | | H), 4.52-4.63 (m, 2 H), 6.81 (d, J = 8 Hz, 2 H), | |
| | | 6.85 (d, J = 8 Hz, 1 H), 7.00 (d, J = 8 Hz, 2 H), 7.06 | |
| | | (dd, J = 8, 5 Hz, 1 H), 7.22 (dd, J = 8, 2 Hz, 1 H), | |
| | | 7.51-7.68 (m, 3 H), 12.68 (br s, 1 H) | |
| 9 | A | 1H NMR (400 MHz, DMSO-d6) δ ppm 1.46- | 570 |
| | | 1.72 (m, 1 H), 1.80-2.06 (m, 1 H), 2.08-3.21 | |
| | | (m, 17 H), 6.21 (tt, J = 56, 4 Hz, 1 H), 6.80 (d, J = 8 | |
| | | Hz, 2 H), 6.87 (d, J = 8 Hz, 1 H), 7.01 (d, J = 8 Hz, 2 | |
| | | H), 7.19 (d, J = 9 Hz, 1 H), 7.26 (dd, J = 8, 3 Hz, 1 | |
| | | H), 7.58 (d, J = 2 Hz, 1 H), 7.76 (dd, J = 8, 2 Hz, 1 | |
| | | H), 7.93 (d, J = 2 Hz, 1 H), 9.81-10.97 (m, 1 H), | |
| | | 11.98-13.66 (m, 1 H) | |
| 10 | | 1H NMR (400 MHz, DMSO-d6) δ ppm 1.42- | 538 |
| | | 1.79 (m, 1 H), 1.83-2.07 (m, 1 H), 2.06-2.38 | |
| | | (m, 9 H), 2.52-2.66 (m, 2 H), 2.88 (t, J = 6 Hz, 2 | |
| | | H), 2.97-3.81 (m, 4 H), 6.21 (br tt, J = 56, 5 Hz, 1 | |
| | | H), 6.79 (d, J = 8 Hz, 2 H), 6.87 (d, J = 8 Hz, 1 H), | |
| | | 6.95 (td, J = 8, 3 Hz, 1 H), 7.01 (d, J = 8 Hz, 2 H), | |
| | | 7.10 (td, J = 10, 3 Hz, 1 H), 7.24 (td, J = 9, 7 Hz, 1 | |
| | | H), 7.75 (dd, J = 8, 2 Hz, 1 H), 7.92 (d, J = 2 Hz, 1 | |
| | | H), 10.28-11.07 (m, 1 H), 12.54-13.25 (m, 1 H) | |
| 11 | B | 1H NMR (400 MHz, DMSO-d6) δ ppm 0.76 (s, 6 | 516 |
| | | H), 1.24 (t, J = 6 Hz, 2 H), 1.42-1.82 (m, 2 H), | |
| | | 1.85-2.26 (m, 9 H), 2.37-2.46 (m, 2 H), 2.69- | |
| | | 2.83 (m, 4 H), 2.92-3.28 (m, 4 H), 3.44-3.72 | |
| | | (m, 2 H), 4.53 (dt, J = 47, 6 Hz, 2 H), 5.42 (br s, 1 | |
| | | H), 6.82 (d, J = 8 Hz, 1 H), 7.00 (d, J = 8 Hz, 2 H), | |
| | | 7.12 (d, J = 8 Hz, 2 H), 7.70 (dd, J = 8, 2 Hz, 1 H), | |
| | | 7.84 (d, J = 2 Hz, 1 H), 9.67-10.87 (m, 1 H), 12.31 | |
| | | (s, 1 H) | |
| 12 | B | 1H NMR (400 MHz, DMSO-d6) δ ppm 1.25- | 566 |
| | | 1.37 (m, 2 H), 1.63-1.85 (m, 6 H), 2.11-2.26 | |
| | | (m, 4 H), 2.27 (s, 3 H), 2.35-2.61 (m, 5 H), 2.83- | |
| | | 3.02 (m, 2 H), 4.44 (dt, J = 48, 6 Hz, 2 H), 6.66 (dd, | |
| | | J = 8, 2 Hz, 2 H), 6.88 (d, J = 8 Hz, 1 H), 6.91 (d, | |
| | | J = 8 Hz, 2 H), 7.25 (td, J = 8, 3 Hz, 1 H), 7.36 (dd, | |
| | | J = 7, 4 Hz, 1 H), 7.51 (d, J = 8 Hz, 1 H), 7.75 (dd, | |
| | | J = 8, 2 Hz, 1 H), 7.92 (d, J = 2 Hz, 1 H), 11.55- | |
| | | 13.99 (m, 1 H) | |
| 13 | B | 1H NMR (400 MHz, DMSO-d6) δ ppm 1.43- | 586 |
| | | 2.31 (m, 9 H), 2.43-2.69 (m, 4 H), 2.80-3.27 | |
| | | (m, 6 H), 4.52 (dt, J = 47, 6 Hz, 2 H), 6.80 (d, J = 7 | |
| | | Hz, 2 H), 6.87 (d, J = 8 Hz, 1 H), 7.04 (d, J = 8 Hz, 2 | |
| | | H), 7.14 (d, J = 8 Hz, 1 H), 7.42 (t, J = 8 Hz, 1 H), | |
| | | 7.56 (d, J = 8 Hz, 1 H), 7.77 (dd, J = 8, 2 Hz, 1 H), | |
| | | 7.93 (d, J = 2 Hz, 1 H), 9.72-10.42 (m, 1 H), 12.70- | |
| | | 13.31 (m, 1 H) | |
| 14 | B | 1H NMR (400 MHz, DMSO-d6) δ ppm 1.27 - | 515 |
| | | 1.42 (m, 1 H), 1.65-1.87 (m, 3 H), 2.02-2.21 | |
| | | (m, 3 H), 2.22-2.29 (m, 2 H), 2.30-2.62 (m, 8 | |
| | | H), 2.84 (t, J = 7 Hz, 2 H), 3.77 (s, 3 H), 4.45 (dt, | |
| | | J = 47, 6 Hz, 2 H), 6.64 (d, J = 9 Hz, 1 H), 6.80 (d, | |
| | | J = 8 Hz, 2 H), 6.84 (d, J = 8 Hz, 1 H), 7.00 (d, J = 8 | |
| | | Hz, 2 H), 7.47 (dd, J = 9, 2 Hz, 1 H), 7.73 (dd, J = 8, | |
| | | 2 Hz, 1 H), 7.89 (d, J = 2 Hz, 1 H), 7.91 (d, J = 2 Hz, | |
| | | 1 H), 12.41-13.33 (m, 1 H) | |
| 15 | B | 1H NMR (400 MHz, DMSO-d6) δ ppm 1.27- | 532 |
| | | 1.39 (m, 1 H), 1.66-1.85 (m, 3 H), 2.00-2.08 | |
| | | (m, 1 H), 2.11-2.24 (m, 6 H), 2.25-2.58 (m, 9 | |
| | | H), 2.80-3.01 (m, 2 H), 4.46 (dt, J = 47, 6 Hz, 2 | |
| | | H), 6.69 (d, J = 8 Hz, 2 H), 6.87 (d, J = 8 Hz, 1 H), | |
| | | 6.94 (d, J = 8 Hz, 2 H), 6.98-7.10 (m, 2 H), 7.25 | |
| | | (d, J = 8 Hz, 1 H), 7.75 (dd, J = 8, 2 Hz, 1 H), 7.91 | |
| | | (d, J = 2 Hz, 1 H), 12.05-13.54 (m, 1 H) | |
| 16 | B | 1H NMR (400 MHz, DMSO-d6) δ ppm 1.27- | 536 |
| | | 1.38 (m, 1 H), 1.65-1.86 (m, 3 H), 2.04 (dd, J = 9, | |
| | | 7 Hz, 1 H), 2.11-2.33 (m, 5 H), 2.36-2.56 (m, 7 | |
| | | H), 2.88 (t, J = 6 Hz, 2 H), 4.46 (dt, J = 47, 6 Hz, 2 | |
| | | H), 6.75 (d, J = 8 Hz, 2 H), 6.88 (d, J = 8 Hz, 1 H), | |
| | | 6.97 (d, J = 8 Hz, 2 H), 7.09 (t, J = 8 Hz, 1 H), 7.21 | |
| | | (td, J = 7, 1 Hz, 1 H), 7.39 (td, J = 7, 1 Hz, 1 H), | |
| | | 7.76 (dd, J = 8, 2 Hz, 1 H), 7.92 (d, J = 2 Hz, 1 H), | |
| | | 12.16-13.57 (m, 1 H) | |
| 17 | B | 1H NMR (400 MHz, DMSO-d6) δ ppm 1.26- | 516 |
| | | 1.38 (m, 1 H), 1.65-1.84 (m, 3 H), 1.97-2.08 | |
| | | (m, 1 H), 2.10-2.20 (m, 7 H), 2.28 (m, 1 H), 2.35- | |
| | | 2.64 (m, 7 H), 2.80-2.99 (m, 2 H), 4.45 (dt, | |
| | | J = 47, 6 Hz, 2 H), 6.68 (d, J = 8 Hz, 2 H), 6.79- | |
| | | 6.89 (m, 2 H), 6.90-7.00 (m, 3 H), 7.05 (ddd, | |
| | | J = 8, 6, 3 Hz, 1 H), 7.73 (dd, J = 8, 2 Hz, 1 H), 7.90 | |
| | | (d, J = 2 Hz, 1 H), 12.28-13.38 (m, 1 H) | |
| 18 | B | 1H NMR (400 MHz, DMSO-d6) δ ppm 1.25- | 570 |
| | | 1.38 (m, 1 H), 1.63-1.84 (m, 3 H), 1.98-2.07 | |
| | | (m, 1 H), 2.12-2.23 (m, 4 H), 2.50 (s, 8 H), 2.80- | |
| | | 3.08 (m, 2 H), 4.45 (dt, J = 48, 7 Hz, 2 H), 6.73 (d, | |
| | | J = 8 Hz, 2 H), 6.86 (d, J = 8 Hz, 1 H), 6.95 (d, J = 8 | |
| | | Hz, 2 H), 7.24 (m, 1 H), 7.34 (m, 1 H), 7.63 (dd, | |
| | | J = 9, 3 Hz, 1 H), 7.74 (dd, J = 8, 2 Hz, 1 H), 7.90 (d, | |
| | | J = 2 Hz, 1 H), 11.90-13.46 (m, 1 H) | |
| 19 | B | 1H NMR (400 MHz, DMSO-d6) δ ppm 1.28- | 520 |
| | | 1.40 (m, 1 H), 1.65-1.85 (m, 3 H), 2.06 (dd, J = 9, | |
| | | 7 Hz, 1 H), 2.10-2.24 (m, 4 H), 2.50 (s, 8 H), | |
| | | 2.87 (t, J = 6 Hz, 2 H), 4.45 (dt, J = 47, 6 Hz, 2 H), | |
| | | 6.75 (d, J = 8 Hz, 2 H), 6.86 (d, J = 8 Hz, 1 H), | |
| | | 6.91(m, 1 H), 6.96 (d, J = 8 Hz, 2 H), 7.10 (td, | |
| | | J = 10, 2 Hz, 1 H), 7.22 (q, J = 8 Hz, 1 H), 7.75 (dd, | |
| | | J = 8, 2 Hz, 1 H), 7.91 (d, J = 2 Hz, 1 H), 12.51- | |
| | | 13.41 (m, 1 H) | |
| 20 | B | 1H NMR (400 MHz, DMSO-d6) δ ppm 1.33 (dq, | 516 |
| | | J = 13, 7 Hz, 1 H), 1.63-1.87 (m, 3 H), 2.04 (s, 3 | |
| | | H), 2.07-2.62 (m, 13 H), 2.81-2.99 (m, 2 H), | |
| | | 4.45 (dt, J = 47, 6 Hz, 2 H), 6.69 (d, J = 8 Hz, 2 H), | |
| | | 6.80-7.01 (m, 5 H), 7.07 (q, J = 7 Hz, 1 H), 7.75 | |
| | | (dd, J = 8, 2 Hz, 1 H), 7.91 (d, J = 2 Hz, 1 H), | |
| | | 11.13-14.28 (m, 1 H) | |
| 21 | B | 1H NMR (400 MHz, DMSO-d6) δ ppm 1.35 (m, 1 | 536 |
| | | H), 1.60-1.89 (m, 3 H), 1.97-2.09 (m, 1 H), 2.10- | |
| | | 2.34 (m, 6 H), 2.36-2.74 (m, 6 H), 2.87-3.01 | |
| | | (m, 2 H), 4.45 (dt, J = 47, 6 Hz, 2 H), 6.75 (d, J = 8 | |
| | | Hz, 2 H), 6.87 (d, J = 8 Hz, 1 H), 6.96 (d, J = 8 Hz, 2 | |
| | | H), 7.03 (d, J = 7 Hz, 1 H), 7.12-7.32 (m, 2 H), | |
| | | 7.75 (dd, J = 8, 2 Hz, 1 H), 7.92 (d, J = 2 Hz, 1 H) | |
| 22 | A | 1H NMR (500 MHz, DMSO-d6) δ ppm 1.92- | 570 |
| | | 2.11 (m, 3 H), 2.13-2.29 (m, 4 H), 2.29-2.41 | |
| | | (m, 1 H), 2.88-2.98 (m, 2 H), 2.99-3.16 (m, 2 | |
| | | H), 3.19-3.90 (m, 6 H), 4.52 (dt, J = 47, 6 Hz, 2 | |
| | | H), 6.84 (d, J = 8 Hz, 2 H), 6.87 (d, J = 8 Hz, 1 H), | |
| | | 7.06 (d, J = 8 Hz, 2 H), 7.18 (m, 1 H), 7.28 (m, 1 | |
| | | H), 7.58 (d, J = 2 Hz, 1 H), 7.76 (dd, J = 8, 2 Hz, 1 | |
| | | H), 7.93 (d, J = 2 Hz, 1 H), 10.28 (br s, 1 H), 12.99 | |
| | | (br s, 1 H) | |
| 23 | A | 1H NMR (500 MHz, DMSO-d6) δ ppm 1.91- | 570 |
| | | 2.10 (m, 3 H), 2.12-2.28 (m, 4 H), 2.29-2.45 | |
| | | (m, 1 H), 2.91-2.98 (m, 2 H), 3.00-3.14 (m, 2 | |
| | | H), 3.19-3.35 (m, 4 H), 3.51-3.84 (m, 2 H), 4.52 | |
| | | (dt, J = 47, 6 Hz, 2 H), 6.83 (d, J = 8 Hz, 2 H), 6.87 | |
| | | (d, J = 8 Hz, 1 H), 7.06 (d, J = 8 Hz, 2 H), 7.20 (d, | |
| | | J = 8 Hz, 1 H), 7.26 (dd, J = 8, 2 Hz, 1 H), 7.58 (d, | |
| | | J = 2 Hz, 1 H), 7.76 (dd, J = 8, 2 Hz, 1 H), 7.93 (d, | |
| | | J = 2 Hz, 1 H), 10.23 (br s, 1 H), 12.92 (br s, 1 H) | |
| 24 | A | 1H NMR (400 MHz, DMSO-d6) δ ppm 1.30- | 552 |
| | | 1.40 (m, 1 H), 1.64-1.85 (m, 3 H), 1.99-2.09 | |
| | | (m, 1 H), 2.10-2.62 (m, 12 H), 2.93 (br s, 2 H), | |
| | | 4.45 (dt, J = 47, 6 Hz, 2 H), 6.76 (d, J = 8 Hz, 2 H), | |
| | | 6.86 (d, J = 8 Hz, 1 H), 6.96 (d, J = 8 Hz, 2 H), 7.16 | |
| | | (m, 1 H), 7.23 (m, 1 H), 7.56 (d, J = 2 Hz, 1 H), | |
| | | 7.74 (dd, J = 8, 2 Hz, 1 H), 7.91 (d, J = 2 Hz, 1 H) | |
| 25 | B | 1H NMR (400 MHz, DMSO-d6) δ ppm 1.44- | 556 |
| | | 1.76 (m, 1 H), 1.79-2.15 (m, 3 H), 2.55-3.56 (m | |
| | | partially hidden, 7 H), 3.16-3.25 (m, 2 H), 3.64 | |
| | | (dd, J = 15, 3 Hz, 1 H), 3.86 (dd, J = 15, 3 Hz, 1 H), | |
| | | 4.53 (dt, J = 46, 5 Hz, 2 H), 6.84 (dd, J = 8, 1 Hz, 1 | |
| | | H), 6.93 (d, J = 8 Hz, 2 H), 7.04-7.13 (m, 3 H), | |
| | | 7.19 (dd, J = 8, 2 Hz, 1 H), 7.59 (t, J = 2 Hz, 1 H), | |
| | | 7.61 (dd, J = 8, 2 Hz, 1 H), 7.93 (d, J = 2 Hz, 1 H), | |
| | | 10.02-10.77 (m, 1 H), 13.08 (br s, 1 H) | |
| 26 | A | 1H NMR (400 MHz, DMSO-d6) δ ppm 1.66- | 588 |
| | | 2.00 (m, 4 H), 2.10-2.30 (m, 4 H), 2.48-3.63 | |
| | | (m, 7 H), 2.90-3.02 (m, 2 H), 4.46 (dt, J = 47, 6 | |
| | | Hz, 2 H), 6.87 (d, J = 8 Hz, 1 H), 6.98 (d, J = 8 Hz, 2 | |
| | | H), 7.20 (m, 1 H), 7.26 (m, 1 H), 7.33 (d, J = 9 Hz, | |
| | | 2 H), 7.58 (d, J = 2 Hz, 1 H), 7.77 (dd, J = 8, 2 Hz, 1 | |
| | | H), 7.94 (d, J = 2 Hz, 1 H) | |
| 27 | B | 1H NMR (500 MHz, DMSO-d6) δ ppm 1.46- | 554 |
| | | 1.72 (m, 1 H), 1.83-2.07 (m, 1 H), 2.11-2.34 | |
| | | (m, 6 H), 2.39-2.46 (m, 1 H), 2.53-2.63 (m | |
| | | partially hidden, 2 H), 2.90-2.96 (m, 2 H), 3.00- | |
| | | 3.65 (m partialy hidden, 6 H), 6.21 (tt, J = 56, 4 Hz, | |
| | | 1 H), 6.76-6.83 (m, 2 H), 6.87 (d, J = 8 Hz, 1 H), | |
| | | 7.00 (d, J = 8 Hz, 2 H), 7.06 (td, J = 8, 3 Hz, 1 H), | |
| | | 7.21 (dd, J = 9, 6 Hz, 1 H), 7.41 (dd, J = 9, 3 Hz, 1 | |
| | | H), 7.75 (dd, J = 8, 2 Hz, 1 H), 7.92 (d, J = 2 Hz, 1 | |
| | | H), 9.85-10.77 (m, 1 H), 12.86 (br s, 1 H) | |
| 28 | B | 1H NMR (500 MHz, DMSO-d6) δ ppm 1.51 (m, 1 | 584 |
| | | H), 1.88 (m, 1 H), 2.11-2.27 (m, 7 H), 2.30 (s, 3 | |
| | | H), 2.53-3.27 (m partially hidden, 10 H), 6.19 (t, | |
| | | J = 58 Hz, 1 H), 6.70 (dd, J = 8, 2 Hz, 2 H), 6.90 (d, | |
| | | J = 8 Hz, 1 H), 6.96 (d, J = 8 Hz, 2 H), 7.25 (t, J = 8 | |
| | | Hz, 1 H), 7.35 (br d, J = 8 Hz, 1 H), 7.51 (d, J = 8 | |
| | | Hz, 1 H), 7.76 (dd, J = 8, 2 Hz, 1 H), 7.93 (d, J = 2 | |
| | | Hz, 1 H), 10.27-11.38 (m, 1 H), 12.10-13.45 | |
| | | (m, 1 H) | |
| 29 | C | 1H NMR (400 MHz, DMSO-d6) δ ppm 1.45- | 524 |
| | | 1.72 (m, 1 H), 1.80-2.25 (m, 7 H), 2.51-2.88 (m | |
| | | partially hidden, 5 H), 2.93-3.24 (m, 4 H), 3.34- | |
| | | 3.67 (m, 2 H), 4.52 (dt, J = 47, 6 Hz, 2 H), 6.46- | |
| | | 6.63 (m, 2 H), 6.73 (d, J = 2 Hz, 1 H), 6.78 (d, J = 8 | |
| | | Hz, 2 H), 6.97 (br d, J = 8 Hz, 2 H), 7.10-7.16 (m, | |
| | | 1 H), 7.21 (dd, J = 8, 2 Hz, 1 H), 7.54 (d, J = 2 Hz, 1 | |
| | | H), 9.45 (s, 1 H), 10.17-10.54 (m, 1 H) | |
| 30 | B | 1H NMR (400 MHz, DMSO-d6) δ ppm 1.32 (m, 1 | 566 |
| | | H), 1.63-1.85 (m, 3 H), 1.94-2.50 (m partially | |
| | | hidden, 16 H), 2.83 (m, 1 H), 2.93-3.08 (m, 1 H), | |
| | | 4.45 (m, 2 H), 6.76 (d, J = 8 Hz, 2 H), 6.86 (d, J = 8 | |
| | | Hz, 1 H), 6.96 (d, J = 8 Hz, 3 H), 7.21-7.29 (m, 2 | |
| | | H), 7.75 (dd, J = 8, 2 Hz, 1 H), 7.90 (d, J = 2 Hz, 1 | |
| | | H) | |
| 31 | C | 1H NMR (400 MHz, DMSO-d6) δ ppm 1.42- | 542 |
| | | 1.74 (m, 1 H), 1.83-2.36 (m, 6 H), 2.40-2.94 (m | |
| | | partially hidden, 6 H), 3.14 (m partially hidden, 2 | |
| | | H), 3.21-3.29 (m, 2 H), 3.40-3.70 (m, 2 H), 6.22 | |
| | | (tt, J = 56, 4 Hz, 1 H), 6.49-6.64 (m, 2 H), 6.73 (d, | |
| | | J = 2 Hz, 1 H), 6.78 (d, J = 8 Hz, 2 H), 6.97 (br d, | |
| | | J = 8 Hz, 2 H), 7.13 (d, J = 8 Hz, 1 H), 7.22 (dd, J = 8, | |
| | | 2 Hz, 1 H), 7.54 (d, J = 2 Hz, 1 H), 9.45 (s, 1 H), | |
| | | 10.31-10.81 (m, 1 H) | |
| 32 | B | 1H NMR (400 MHz, DMSO-d6) δ ppm 1.50 (m, 1 | 548 |
| | | H), 1.89 (m, 1 H), 2.02-2.28 (m, 10 H), 2.46- | |
| | | 2.62 (m partially hidden, 9 H), 2.96 (br t, J = 7 Hz, | |
| | | 2 H), 3.00-3.27 (m partially hidden, 2 H), 6.17 ( | |
| | | t, J = 56 Hz, 1 H), 6.68 (m, J = 8 Hz, 2 H), 6.77- | |
| | | 6.88 (m, 3 H), 6.95 (m, 2 H), 7.74 (dd, J = 8, 2 Hz, | |
| | | 1 H), 7.93 (d, J = 2 Hz, 1 H), 9.80-11.49 (m, 1 H), | |
| | | 11.71-13.45 (m, 1 H) | |
| 33 | B | 1H NMR (400 MHz, DMSO-d6) δ ppm 1.33 (m, 1 | 570 |
| | | H), 1.73 (m, 1 H), 1.85-2.12 (m, 3 H), 2.13-2.46 | |
| | | (m partially hidden, 10 H), 2.79-3.04 (m, 4 H), | |
| | | 6.06 (ttd, J = 56, 5, 1 Hz, 1 H), 6.73 (d, J = 8 Hz, 2 | |
| | | H), 6.87 (d, J = 8 Hz, 1 H), 6.92 (d, J = 8 Hz, 2 H), | |
| | | 7.18 (d, J = 7 Hz, 1 H), 7.38-7.45 (m, 2 H), 7.68- | |
| | | 7.77 (m, 2 H), 7.90 (s, 1 H) | |
| 34 | B | 1H NMR (400 MHz, DMSO-d6) δ ppm 1.44- | 584 |
| | | 1.75 (m, 2 H), 1.82-2.32 (m, 6 H), 2.46 (s, 6 H), | |
| | | 2.53-3.25 (m partially hidden, 7 H), 3.41-3.66 | |
| | | (m, 1 H), 6.21 (tt, J = 56, 5 Hz, 1 H), 6.79 (d, J = 8 | |
| | | Hz, 2 H), 6.86 (d, J = 8 Hz, 1 H), 6.94 (br d, J = 6 | |
| | | Hz, 1 H), 7.00 (br d, J = 8 Hz, 2 H), 7.22-7.30 (m, | |
| | | 2 H), 7.75 (dd, J = 8, 2 Hz, 1 H), 7.91 (d, J = 2 Hz, 1 | |
| | | H), 10.14-10.92 (m, 1 H), 12.85 (br s, 1 H) | |
| 35 | B | 1H NMR (400 MHz, DMSO-d6) δ ppm 1.56 (m, 1 | 530 |
| | | H), 1.83-2.13 (m, 6 H), 2.16 (s, 6 H), 2.17-2.25 | |
| | | (m, 2 H), 2.54-2.64 (m partailly hidden, 5 H), | |
| | | 2.96 (br t, J = 7 Hz, 2 H), 3.07-3.23 (m, 2 H), 4.51 | |
| | | (dt, J = 45, 7 Hz, 2 H), 6.69 (m, J = 8 Hz, 2 H), 6.79- | |
| | | 6.87 (m, 3 H), 6.96 (m, J = 8 Hz, 2 H), 7.46-7.60 | |
| | | (m, 1 H), 7.74 (dd, J = 8, 2 Hz, 1 H), 7.93 (d, J = 2 | |
| | | Hz, 1 H), 10.10 (br s, 1 H), 12.83 (br s, 1 H) | |
| 36 | C | 1H NMR (400 MHz, DMSO-d6) δ ppm 1.66 (m, 1 | 538 |
| | | H), 1.92-2.14 (m, 3 H), 2.51-2.76 (m, 9 H), 3.06 | |
| | | (t, J = 8 Hz, 2 H), 3.12-3.24 (m, 2 H), 4.52 (dt, | |
| | | J = 47, 5 Hz, 2 H), 6.77 (d, J = 8 Hz, 1 H), 6.95- | |
| | | 7.02 (m, 2 H), 7.05-7.18 (m, 4 H), 7.39 (d, J = 2 | |
| | | Hz, 1 H), 7.65 (d, J = 8 Hz, 1 H), 7.81 (s, 1 H), | |
| | | 10.19 (s, 1 H), 12.04 (s, 1 H) | |
| 37 | B | 1H NMR (400 MHz, DMSO-d6) δ ppm 1.61 (m, 1 | 518 |
| | | H), 1.84-2.21 (m, 5 H), 2.23-2.31 (m, 2 H), 2.55- | |
| | | 2.76 (m partially hidden, 5 H), 2.81-2.90 (m, 2 | |
| | | H), 2.97-3.23 (m partially hidden, 4 H), 4.52 (dt, | |
| | | J = 47, 6 Hz, 2 H), 6.81 (d, J = 8 Hz, 2 H), 6.85 (d, | |
| | | J = 8 Hz, 1 H), 7.02 (d, J = 8 Hz, 2 H), 7.14-7.18 | |
| | | (m, 2 H), 7.23-7.27 (m, 2 H), 7.74 (dd, J = 8, 2 | |
| | | Hz, 1 H), 7.91 (d, J = 2 Hz, 1 H), 10.28 (br s, 1 H), | |
| | | 12.78 (s, 1 H) | |
| 38 | B | 1H NMR (500 MHz, DMSO-d6) δ ppm 1.59 (m, 1 | 552 |
| | | H), 1.82-2.15 (m, 5 H), 2.25-2.35 (m, 2 H), 2.49- | |
| | | 2.79 (m, 5 H), 2.88 (br t, J = 7 Hz, 2 H), 3.05- | |
| | | 3.25 (m, 3 H), 3.38-3.62 (m, 1 H), 4.52 (dt, J = 48, | |
| | | 4 Hz, 2 H), 6.81 (d, J = 8 Hz, 2 H), 6.87 (d, J = 8 Hz, | |
| | | 1 H), 7.02 (d, J = 8 Hz, 2 H), 7.37 (d, J = 8 Hz, 2 H), | |
| | | 7.54 (d, J = 8 Hz, 2 H), 7.76 (dd, J = 8, 2 Hz, 1 H), | |
| | | 7.93 (d, J = 2 Hz, 1 H), 10.02-10.65 (m, 1 H), | |
| | | 12.89 (br s, 1 H) | |
| 39 | B | 1H NMR (500 MHz, DMSO-d6) δ ppm 1.48 - | 552 |
| | | 1.73 (m, 1 H), 1.80-2.10 (m, 3 H), 2.17 (quin, | |
| | | J = 7 Hz, 2 H), 2.29-2.36 (m, 2 H), 2.44-2.77 (m | |
| | | partially hidden, 5 H), 2.88 (t, J = 7 Hz, 2 H), 2.93- | |
| | | 3.71 (m partially hidden, 4 H), 4.53 (dt, J = 47, 6 | |
| | | Hz, 2 H), 6.81 (d, J = 7 Hz, 2 H), 6.87 (d, J = 8 Hz, 1 | |
| | | H), 7.02 (d, J = 8 Hz, 2 H), 7.36 (s, 1 H), 7.44- | |
| | | 7.56 (m, 3 H), 7.75 (dd, J = 8, 2 Hz, 1 H), 7.92 (d, | |
| | | J = 2 Hz, 1 H), 9.77-10.65 (m, 1 H), 12.87 (br s, 1 | |
| | | H) | |
| 40 | B | 1H NMR (400 MHz, DMSO-d6) δ ppm 1.64 (t, | 518 |
| | | J = 8 Hz, 1 H), 1.89-2.19 (m, 5 H), 2.28 (t, J = 7 | |
| | | Hz, 2 H), 2.44-2.77 (m partially hidden , 5 H), | |
| | | 2.86 (t, J = 7 Hz, 2 H), 3.32 (m partially hidden, 4 | |
| | | H), 4.53 (dt, J = 47, 6 Hz, 2 H), 6.82 (d, J = 8 Hz, 2 | |
| | | H), 6.87 (d, J = 8 Hz, 1 H), 7.03 (d, J = 8 Hz, 2 H), | |
| | | 7.11-7.26 (m, 4 H), 7.75 (dd, J = 8, 2 Hz, 1 H), | |
| | | 7.91 (d, J = 2 Hz, 1 H), 10.48 (br s, 1 H), 12.81 (br | |
| | | s, 1 H) | |
| 41 | B | 1H NMR (400 MHz, DMSO-d6) δ ppm 0.73 (d, | 566 |
| | | J = 7 Hz, 3 H) 1.22-1.32 (m, 1 H) 1.65-1.80 (m, | |
| | | 3 H) 1.94-2.05 (m, 2 H) 2.20-2.30 (m, 2 H) 2.37- | |
| | | 2.48 (m, 8 H partially hidden) 2.79-2.91 (m, 1 | |
| | | H) 2.97-3.10 (m, 1 H) 4.44 (dt, J = 48 Hz, J = 6 Hz, | |
| | | 2 H) 6.83 (d, J = 8 Hz, 2 H) 6.91 (d, J = 8 Hz, 2 H) | |
| | | 6.95 (d, J = 8 Hz, 1 H) 7.35 (d, J = 8 Hz, 2 H) 7.53 | |
| | | (d, J = 8 Hz, 2 H) 7.74 (dd, 1 H) 7.90 (d, J = 2 Hz, 1 | |
| | | H) | |
| 42 | B | 1H NMR (400 MHz, DMSO-d6) δ ppm 0.74 (d, | 566 |
| | | J = 7 Hz, 3 H) 1.24-1.34 (m, 1 H) 1.67-1.80 (m, | |
| | | 3 H) 2.01 (m, 2 H) 2.20-2.30 (m, 2 H) 2.36-2.48 | |
| | | (m, 8 H, partially hidden) 2.8-2.9 (m, 1 H) 3.03- | |
| | | 3.11 (m, 1 H) 4.44 (dt, J = 47 Hz, 6 Hz, 2H) 6.81- | |
| | | 6.86 (m, 2 H) 6.91 (d, J = 8 Hz, 2 H) 6.94 (d, J = 8 | |
| | | Hz, 1 H) 7.36 (d, J = 8 Hz, 2 H) 7.54 (d, J = 8 Hz, 2 | |
| | | H) 7.75 (dd, J = 8 Hz, 2 Hz, 1 H) 7.91 (d, J = 2 Hz, 1 | |
| | | H) | |
| 43 | E | 1H NMR (500 MHz, DMSO-d6) δ ppm 1.33 (m, 1 | 542 |
| | | H), 1.69-1.84 (m, 3 H), 2.04 (m, 1 H), 2.24-2.62 | |
| | | (m partially hidden, 10 H), 3.11 (td, J = 12, 5 Hz, 1 | |
| | | H), 3.42 (m, 1 H), 4.46 (dt, J = 485, 6 Hz, 2 H), | |
| | | 6.33 (d, J = 3 Hz, 1 H), 6.70 (dd, J = 8, 3 Hz, 1 H), | |
| | | 6.74 (d, J = 8 Hz, 2 H), 6.97 (d, J = 8 Hz, 2 H), 7.03- | |
| | | 7.09 (m,1 H), 7.21 (br d, J = 8 Hz, 1 H), 7.44 (d, | |
| | | J = 8 Hz, 1 H), 7.63 (d, J = 2 Hz, 1 H), 9.59 (s, 1 H) | |
| 44 | E | 1H NMR (500 MHz, DMSO-d6) δ ppm 1.32 (m, 1 | 508 |
| | | H), 1.66-1.85 (m, 3 H), 2.06 (m, 1 H), 2.22-2.57 | |
| | | (m partially hidden, 10 H), 3.10 (td, J = 12.1, 4.6 | |
| | | Hz, 1 H), 3.44 (m, 1 H), 4.45 (dt, J = 47, 6 Hz, 2 | |
| | | H), 6.33 (d, J = 3 Hz, 1 H), 6.69 (dd, J = 8, 3 Hz, 1 | |
| | | H), 6.75 (d, J = 8 Hz, 2 H), 6.93 (d, J = 8.Hz, 2 H), | |
| | | 7.06 (br d, J = 8 Hz, 1 H), 7.10 (t, J = 8 Hz, 1 H), | |
| | | 7.20 (t, J = 8 Hz, 1 H), 7.44 (d, J = 8 Hz, 1 H), 7.46 | |
| | | (d, J = 8 Hz, 1 H), 9.57 (s, 1 H) | |

The examples which follow describe the preparation of some compounds of formula (I) described herein. The numbers of the compounds exemplified below match those given in the Table 1 above. All reactions are performed under inert atmosphere, unless otherwise stated.

In the following examples, when the source of the starting products is not specified, it should be understood that said products are known compounds.

### INTERMEDIATES

### Intermediate 1: 3-(4-Bromobenzyl)-1-(3-fluoropropyl)pyrrolidine, Isomer 1 and Isomer 2

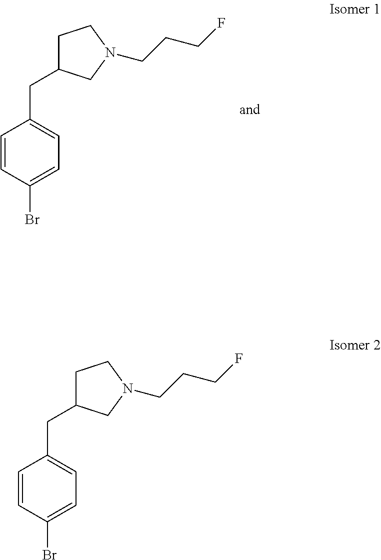 [SMILES: FCCCN1CCC(Cc2ccc(Br)cc2)C1 ; FCCCN1CCC(Cc2ccc(Br)cc2)C1]

### Step 1: Tert-butyl 3-(4-bromobenzyl)pyrrolidine-1-carboxylate, Isomer 1 and Isomer 2

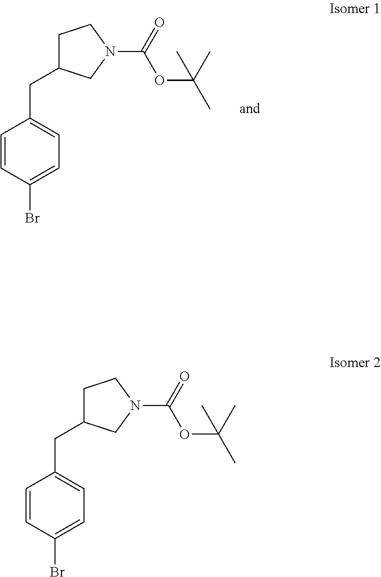 [SMILES: CC(C)(C)OC(=O)N1CCC(Cc2ccc(Br)cc2)C1 ; CC(C)(C)OC(=O)N1CCC(Cc2ccc(Br)cc2)C1]

To a solution of commercially available 3-(4-bromobenzyl)pyrrolidine (35.0 g, 145 mmol) and TEA (29.5 g, 292 mmol) in DCM (250 ml) was added DMAP (1.78 g, 14.57 mmol) and Boc₂O (33.4 g, 153 mmol) at RT and the suspension was stirred at RT for 12 hours. The reaction mixture was quenched by addition of water (300 ml) and extracted with DCM (100 ml). The organic phase was washed with brine (200 ml), dried over Na₂SO₄ filtered and concentrated under reduced pressure and the residue obtained was purified by flash chromatography, eluting with a gradient of petroleum ether/ethyl acetate: from 98/02 to 50/50 to give 37 g (73%) of tert-butyl 3-(4-bromobenzyl)pyrrolidine-1-carboxylate as a light yellow oil.

The mixture of isomers of tert-butyl 3-(4-bromobenzyl)pyrrolidine-1-carboxylate (37 g, 107 mmol) was separated by preparative SFC (column: DAICEL CHIRALCEL OJ (250×50 mm, 10 μm); supercritical CO₂ 80%/[0.1% NH₄OH EtOH]20%) to give 17 g of tert-butyl 3-(4-bromobenzyl)pyrrolidine-1-carboxylate Isomer 1 and 17 g of tert-butyl 3-(4-bromobenzyl)pyrrolidine-1-carboxylate Isomer 2.

LC/MS (m/z, MH+): 340

### Step 2: 3-(4-Bromobenzyl)pyrrolidine hydrochloride, Isomer 1

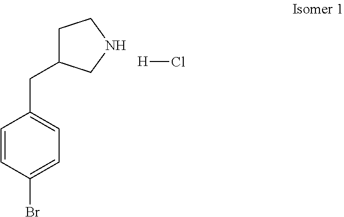

To a solution of tert-butyl 3-(4-bromobenzyl)pyrrolidine-1-carboxylate Isomer 1 (17.0 g, 50.0 mmol) in EtOAc (160 ml) was added a 4N solution of HCl in dioxane (50 ml, 200 mmol) and the mixture was stirred at RT for 2 hours. The solution was concentrated under vacuum to give compound 17 g (crude) of 3-(4-bromobenzyl)pyrrolidine hydrochloride, Isomer 1.

LC/MS (m/z, MH+): 240

### Step 3: 3-(4-Bromobenzyl)-1-(3-fluoropropyl)pyrrolidine, Isomer 1

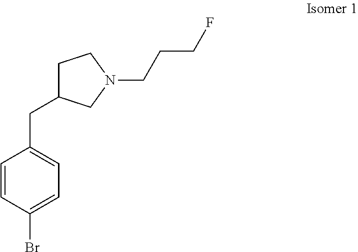 [SMILES: FCCCN1CCC(Cc2ccc(Br)cc2)C1]

To a mixture of 3-(4-bromobenzyl)pyrrolidine hydrochloride, Isomer 1 (12 g, 43.4 mmol) and K₂CO₃ in DMF (84 ml) was added 1-fluoro-3-iodo-propane (8.56 g, 45.55 mmol) and the mixture was heated to 80° C. for 2 hours. The reaction mixture was quenched by addition of water (400 ml) and extracted twice with EtOAc (200 ml). The combined organic phases were washed with brine (200 ml), dried over Na₂SO₄, filtered, concentrated under reduced pressure and the residue obtained was purified by flash chromatography eluting with a gradient of DCM/MeOH from 100/00 to 90/10 to give 10 g (76%) of 3-(4-bromobenzyl)-1-(3-fluoropropyl)pyrrolidine, Isomer 1.

LC/MS (m/z, MH+): 300

### Step 2′: 3-(4-Bromobenzyl)pyrrolidine hydrochloride, Isomer 2

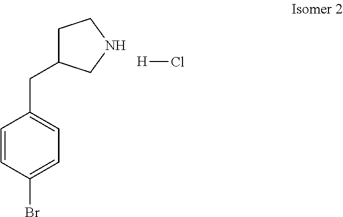

Step 2′ of Intermediate 1 Isomer 2 was prepared following a similar procedure to that of step 2 of Intermediate 1 Isomer 1 from tert-butyl 3-(4-bromobenzyl)pyrrolidine-1-carboxylate Isomer 2 to give 16 g (crude) of 3-(4-bromobenzyl)pyrrolidine hydrochloride, Isomer 2.

LC/MS (m/z, MH+): 240

### Step 3′: 3-(4-Bromobenzyl)-1-(3-fluoropropyl)pyrrolidine, Isomer 2

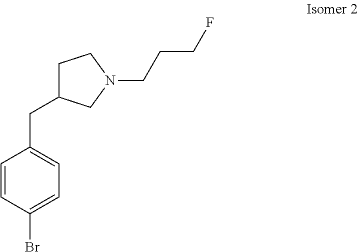 [SMILES: FCCCN1CCC(Cc2ccc(Br)cc2)C1]

Step 3′ of Intermediate 1 Isomer 2 was prepared following a similar procedure to that of step 3 of Intermediate 1 Isomer 1 from 3-(4-bromobenzyl)pyrrolidine hydrochloride, Isomer 2 to give 9.95 g (55%) of 3-(4-bromobenzyl)-1-(3-fluoropropyl)pyrrolidine, Isomer 2.

LC/MS (m/z, MH+): 300

### Intermediate 2: Methyl 9-(4,4,5,5-tetramethyl-1,3,2-dioxaborolan-2-yl)-6,7-dihydro-5H-benzo[7]annulene-3-carboxylate

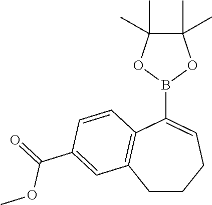

A mixture of methyl 9-(((trifluoromethyl)sulfonyl)oxy)-6,7-dihydro-5H-benzo[7]annulene-3-carboxylate (15 g, 42.82 mmol) (prepared according to WO2017140669), Pd(PPh₃)₂Cl₂ (1.53 g, 2.14 mmol), PPh₃ (674.87 mg, 2.57 mmol), bis(pinacolato)diboron (144.08 g, 52.67 mmol) and PhOK (8.04 g, 60.80 mmol) in toluene (150 ml) was heated to 75° C. for 1.5 hours. The yellow suspension becomes orange then brown. After cooling to room temperature, DCM (150 ml) and water (150 ml) were added, and decantation was done by hydrophobic column. The organic phase was concentrated under reduced pressure. The residue obtained was purified by flash chromatography, eluting with a gradient of heptane/DCM: from 85/15 to 20/80 to give 10.1 g (72%) of methyl 9-(4,4,5,5-tetramethyl-1,3,2-dioxaborolan-2-yl)-6,7-dihydro-5H-benzo[7]annulene-3-carboxylate as a white solid.

LC/MS (m/z, MH+): 329

### Intermediate 3: Methyl 8-(2,4-dichlorophenyl)-9-(4,4,5,5-tetramethyl-1,3,2-dioxaborolan-2-yl)-6,7-dihydro-5H-benzo[7]annulene-3-carboxylate

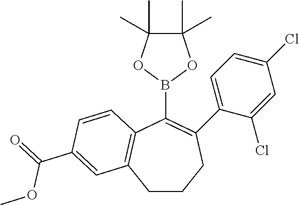 [SMILES: COC(=O)c1ccc2c(c1)CCCC(c1ccc(Cl)cc1Cl)=C2B1OC(C)(C)C(C)(C)O1]

Intermediate 3 was prepared following a similar procedure to that of Intermediate 2 from methyl 8-(2,4-dichlorophenyl)-9-(((trifluoromethyl)sulfonyl)oxy)-6,7-dihydro-5H-benzo[7]annulene-3-carboxylate (prepared according to WO2020/049153) to give 4.23 g (30% yield) of methyl 8-(2,4-dichlorophenyl)-9-(4,4,5,5-tetramethyl-1,3,2-dioxaborolan-2-yl)-6,7-dihydro-5H-benzo[7]annulene-3-carboxylate as a white solid.

LC/MS (m/z, MH+): 473

### Intermediate 4: 3-[(4-Bromophenyl)methyl]-3-fluoro-1-(3-fluoropropyl)pyrrolidine Isomer 1 and Isomer 2

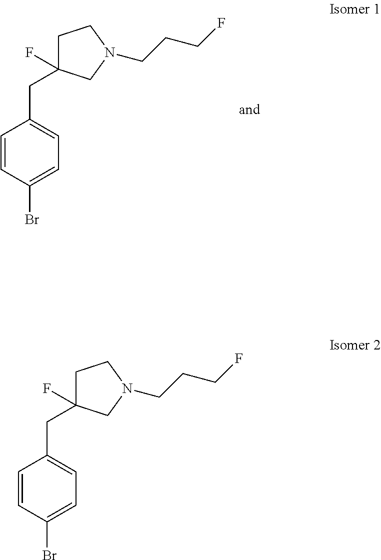 [SMILES: FCCCN1CCC(F)(Cc2ccc(Br)cc2)C1 ; FCCCN1CCC(F)(Cc2ccc(Br)cc2)C1]

### Step 1: Tert-butyl 3-(4-bromobenzoyl)-3-fluoro-pyrrolidine-1-carboxylate

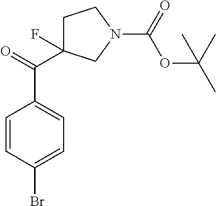 [SMILES: CC(C)(C)OC(=O)N1CCC(F)(C(=O)c2ccc(Br)cc2)C1]

To a solution of commercially available tert-butyl 3-(4-bromobenzoyl)pyrrolidine-1-carboxylate (130 g, 367 mmol) in THE (1.00 L) was added LiHMDS (1 M, 514 ml) at −70° C. The reaction mixture was stirred at −70° C. for 1 hour. N-(Benzenesulfonyl)-N-fluoro-benzenesulfonamide (150 g, 477 mmol) in THE (500 ml) was added to the mixture above at −70° C. The reaction mixture was stirred at −70° C. for 1 hour then it was poured into aqueous NH₄Cl solution (500 ml), extracted with EtOAc 900 ml (3×300 ml), dried over Na₂SO₄, filtered and concentrated under reduced pressure and the residue obtained was purified by flash chromatography eluting with a gradient of Petroleum ether/EtOAc from 10/1 to 4/1 to give 130 g (95%) of tert-butyl 3-(4-bromobenzoyl)-3-fluoro-pyrrolidine-1-carboxylate.

LC/MS (m/z, MH+): 372

### Step 2: Tert-butyl 3-[(4-bromophenyl)-hydroxy-methyl]-3-fluoro-pyrrolidine-1-carboxylate

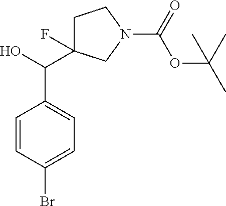 [SMILES: CC(C)(C)OC(=O)N1CCC(F)(C(O)c2ccc(Br)cc2)C1]

To a solution of tert-butyl 3-(4-bromobenzoyl)-3-fluoro-pyrrolidine-1-carboxylate (130 g, 349 mmol) in MeOH (1 L) was added NaBH₄ (19.8 g, 524 mmol) at 0° C. The mixture was stirred at 25° C. for 2 hours. The reaction mixture was quenched by addition of water (20 ml) at 0° C., and then diluted with water (400 ml) and the organic phase was removed. The aqueous layer was extracted with EtOAc (3×400 ml), dried over Na₂SO₄, filtered and concentrated under reduced pressure to give 130 g (crude) of tert-butyl 3-[(4-bromophenyl)-hydroxy-methyl]-3-fluoro-pyrrolidine-1-carboxylate as light yellow oil.

LC/MS (m/z, MH+): 374

### Step 3: Tert-butyl 3-[(4-bromophenyl)-methylsulfanylcarbothioyloxy-methyl]-3-fluoro-pyrrolidine-1-carboxylate

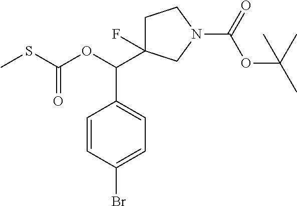 [SMILES: CSC(=O)OC(c1ccc(Br)cc1)C1(F)CCN(C(=O)OC(C)(C)C)C1]

To a solution of tert-butyl 3-[(4-bromophenyl)-hydroxy-methyl]-3-fluoro-pyrrolidine-1-carboxylate (86 g, 230 mmol) and imidazole (78.2 mg, 1.15 mmol) in THE (900 ml) was added NaH (13.8 g, 345 mmol, 60% purity) at 10° C. The reaction mixture was stirred at 10° C. for 30 minutes. Carbon disulfide (35 g, 460 mmol, 27.8 ml) was added to the mixture and the reaction mixture was stirred at 25° C. for 30 minutes. Methyl iodide (58.7 g, 414 mmol, 25.8 ml) was added to the above mixture and the reaction mixture was stirred at RT for 30 minutes. The reaction mixture was poured into NH₄Cl solution (200 ml). The organic phase was separated, washed with EtOAc (3×200 ml), dried over Na₂SO₄, filtered, concentrated under reduced pressure and the residue obtained was purified by flash chromatography eluting with a gradient of Petroleum ether/EtOAc from 90/10 to 70/30 to give 102 g (96%) of tert-butyl 3-[(4-bromophenyl)-methylsulfanylcarbothioyloxy-methyl]-3-fluoro-pyrrolidine-1-carboxylate as light yellow oil.

LC/MS (m/z, MH+): 374

### Step 4: Tert-butyl 3-[(4-bromophenyl)methyl]-3-fluoro-pyrrolidine-1-carboxylate

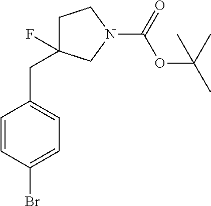 [SMILES: CC(C)(C)OC(=O)N1CCC(F)(Cc2ccc(Br)cc2)C1]

To a solution of tert-butyl 3-[(4-bromophenyl)-methylsulfanylcarbothioyloxy-methyl]-3-fluoro-pyrrolidine-1-carboxylate (25.5 g, 54.9 mmol) in toluene (250 ml) was added tributylstannane (29.6 g, 102 mmol, 26.9 ml) and AIBN (721 mg, 4.39 mmol). The reaction mixture was stirred at 90° C. for 2 hours. After cooling to RT, the reaction mixture was poured into water (1 L), extracted with EtOAc (3×100 ml), dried over Na₂SO₄, filtered, concentrated under reduced pressure and the residue obtained was purified by flash chromatography eluting with a gradient of Petroleum ether/EtOAc from 90/10 to 70/30 to give 17.25 g (88%) of tert-butyl 3-[(4-bromophenyl)methyl]-3-fluoro-pyrrolidine-1-carboxylate as light yellow solid.

LC/MS (m/z, MH+): 358

### Step 5: 3-[(4-Bromophenyl)methyl]-3-fluoro-pyrrolidine, hydrochloride

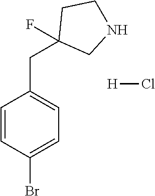

Step 5 of Intermediate 4 was prepared following a similar procedure to that of step 2 of Intermediate 1 Isomer 1 from tert-butyl 3-[(4-bromophenyl)methyl]-3-fluoro-pyrrolidine-1-carboxylate to give 32 g (crude) of 3-[(4-bromophenyl)methyl]-3-fluoro-pyrrolidine, hydrochloride.

LC/MS (m/z, MH+): 240

### Step 6: 3-[(4-Bromophenyl)methyl]-3-fluoro-1-(3-fluoropropyl)pyrrolidine

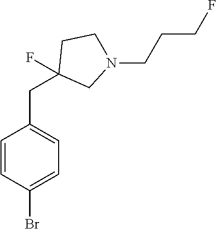 [SMILES: FCCCN1CCC(F)(Cc2ccc(Br)cc2)C1]

To a solution of 3-[(4-bromophenyl)methyl]-3-fluoro-pyrrolidine, hydrochloride (32.0 g, 109 mmol) in THE (220 ml) was added NaOH (5 M, 109 ml) and 1-fluoro-3-iodo-propane (20.4 g, 109 mmol). The reaction mixture was stirred at 70° C. for 2 hours. The reaction mixture was poured into water (100 ml), extracted with EtOAc (300 ml), dried over Na₂SO₄, filtered, concentrated under reduced pressure and the residue obtained was purified by flash chromatography eluting with a gradient of DCM/MeOH from 98/02 to 90/10 to give 28 g (crude) which was further purified by preparative HPLC (column: Agela DuraShell C18 250×70 mm, 10 μm; mobile phase: gradient of MeCN in water (0.05% NH₄OH+10 mM NH₄HCO₃) from 43% to 67% in 20 min) to give 12 g (35%) of 3-[(4-bromophenyl)methyl]-3-fluoro-1-(3-fluoropropyl)pyrrolidine.

LC/MS (m/z, MH+): 318

### Step 7: 3-[(4-Bromophenyl)methyl]-3-fluoro-1-(3-fluoropropyl)pyrrolidine Isomer 1 and Isomer 2

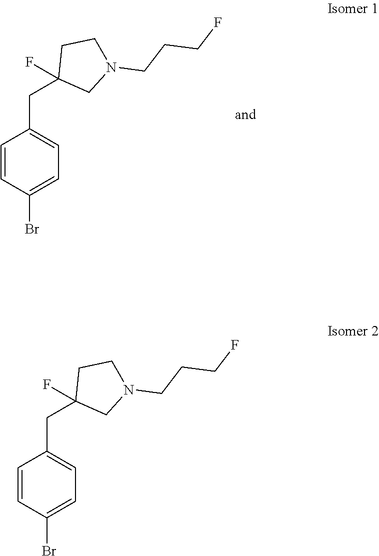 [SMILES: FCCCN1CCC(F)(Cc2ccc(Br)cc2)C1 ; FCCCN1CCC(F)(Cc2ccc(Br)cc2)C1]

The mixture of isomers of 3-[(4-bromophenyl)methyl]-3-fluoro-1-(3-fluoropropyl)pyrrolidine was separated by preparative SFC (column: DAICEL CHIRALPAK IG (250×30 mm,10 μm); supercritical CO₂ 75%/MeOH 25%) to give 5 g of 3-[(4-bromophenyl)methyl]-3-fluoro-1-(3-fluoropropyl)pyrrolidine Isomer 1 and 5 g of 3-[(4-bromophenyl)methyl]-3-fluoro-1-(3-fluoropropyl)pyrrolidine Isomer 2.

LC/MS (m/z, MH+): 318

### Intermediate 5: (4-Bromophenyl)-[1-(3-fluoropropyl)pyrrolidin-3-yl]methanone, racemic mixture

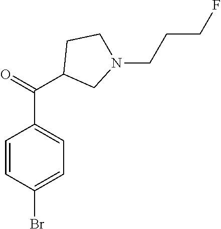 [SMILES: O=C(c1ccc(Br)cc1)C1CCN(CCCF)C1]

### Step 1: (4-Bromophenyl)-pyrrolidin-3-yl-methanone, hydrochloride, racemic mixture

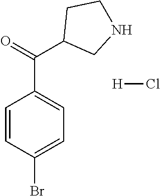

Step 1 of Intermediate 5 was prepared following a similar procedure to that of step 2 of Intermediate 1 Isomer 1 from tert-butyl 3-(4-bromobenzoyl)pyrrolidine-1-carboxylate, racemic mixture to give 76 g (crude) of (4-bromophenyl)-pyrrolidin-3-yl-methanone hydrochloride, racemic mixture.

LC/MS (m/z, MH+): 254

### Step 2: (4-Bromophenyl)-[1-(3-fluoropropyl)pyrrolidin-3-yl]methanone, racemic mixture

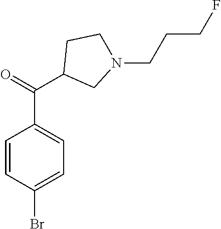 [SMILES: O=C(c1ccc(Br)cc1)C1CCN(CCCF)C1]

Step 2 of Intermediate 5 was prepared following a similar procedure to that of step 6 of Intermediate 4 from (4-bromophenyl)-pyrrolidin-3-yl-methanone hydrochloride, racemic mixture to give 28 g (93%) of (4-bromophenyl)-[1-(3-fluoropropyl)pyrrolidin-3-yl]methanone, racemic mixture.

LC/MS (m/z, MH+): 314

### Intermediate 6: 3-[(4-Bromophenyl)-difluoro-methyl]-1-(3-fluoropropyl)pyrrolidine Isomer 1 and Isomer 2

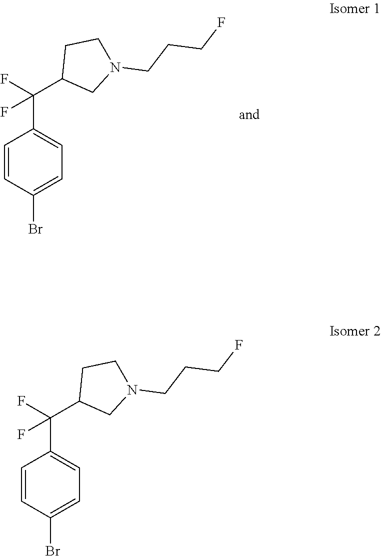 [SMILES: FCCCN1CCC(C(F)(F)c2ccc(Br)cc2)C1 ; FCCCN1CCC(C(F)(F)c2ccc(Br)cc2)C1]

### Step 1: Tert-butyl 3-[(4-bromophenyl)-difluoro-methyl]pyrrolidine-1-carboxylate, racemic mixture

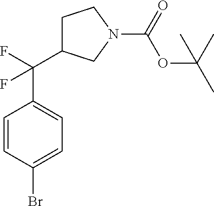 [SMILES: CC(C)(C)OC(=O)N1CCC(C(F)(F)c2ccc(Br)cc2)C1]

To a solution of tert-butyl 3-(4-bromobenzoyl)pyrrolidine-1-carboxylate, racemic mixture (20 g, 56.5 mmol) was added bis(2-methoxyethyl)aminosulfur trifluoride BAST (100 ml). The mixture was stirred at 70° C. for 12 hours. The reaction mixture was diluted with NaHCO₃ (1000 ml) and extracted with EtOAc (300 ml). The organic phase was washed with brine, dried over Na₂SO₄, filtered, concentrated under reduced pressure and the residue obtained was purified by flash chromatography eluting with a gradient of Petroleum ether/Ethyl acetate from 98/02 to 00/100 to give 19 g (89%) of tert-butyl 3-[(4-bromophenyl)-difluoro-methyl]pyrrolidine-1-carboxylate, racemic mixture.

LC/MS (m/z, MH+): 376

### Step 2: 3-[(4-Bromophenyl)-difluoro-methyl]pyrrolidine hydrochloride, racemic mixture

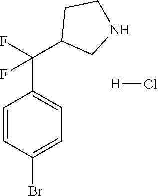

Step 2 of Intermediate 6 was prepared following a similar procedure to that of step 2 of Intermediate 1 Isomer 1 from tert-butyl 3-[(4-bromophenyl)-difluoro-methyl]pyrrolidine-1-carboxylate, racemic mixture to give 20 g (99%) of 3-[(4-bromophenyl)-difluoro-methyl]pyrrolidine, hydrochloride, racemic mixture.

LC/MS (m/z, MH+): 276

### Step 3: 3-[(4-Bromophenyl)-difluoro-methyl]-1-(3-fluoropropyl)pyrrolidine, racemic

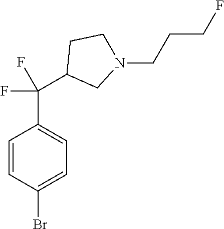 [SMILES: FCCCN1CCC(C(F)(F)c2ccc(Br)cc2)C1]

Step 3 of Intermediate 6 was prepared following a similar procedure to that of step 3 of Intermediate 1 Isomer 1 from 3-[(4-bromophenyl)-difluoro-methyl]pyrrolidine hydrochloride, racemic mixture to give 15 g (62%) of 3-[(4-bromophenyl)-difluoro-methyl]-1-(3-fluoropropyl)pyrrolidine, racemic mixture.

LC/MS (m/z, MH+): 336

### Step 4: 3-[(4-Bromophenyl)-difluoro-methyl]-1-(3-fluoropropyl)pyrrolidine Isomer 1 and Isomer 2

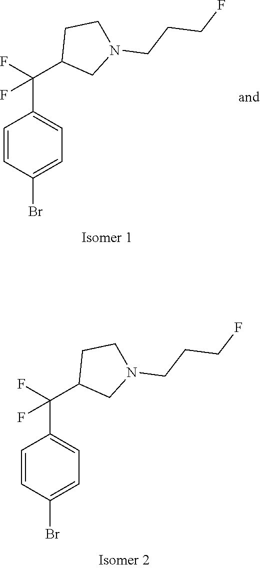 [SMILES: FCCCN1CCC(C(F)(F)c2ccc(Br)cc2)C1 ; FCCCN1CCC(C(F)(F)c2ccc(Br)cc2)C1]

The mixture of isomers of 3-[(4-bromophenyl)-difluoro-methyl]-1-(3-fluoropropyl)pyrrolidine was separated by preparative SFC (column: DAICEL CHIRALPAK AD (250×50 mm, 10 μm); supercritical CO₂ 85%/[0.1% NH₄OH MeOH]15%) to give 5.2 g (21%) of 3-[(4-bromophenyl)-difluoro-methyl]-1-(3-fluoropropyl)pyrrolidine Isomer 1 and 5.2 g (21%) of 3-[(4-bromophenyl)-difluoro-methyl]-1-(3-fluoropropyl)pyrrolidine Isomer 2.

LC/MS (m/z, MH+): 336

### Intermediate 7: Methyl 8-bromo-9-(4-((1-(3-fluoropropyl)pyrrolidin-3-yl)methyl)phenyl)-6,7-dihydro-5H-benzo[7]annulene-3-carboxylate Isomer 1

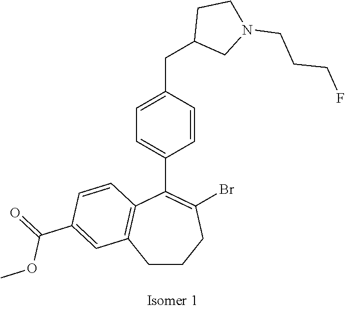 [SMILES: COC(=O)c1ccc2c(c1)CCCC(Br)=C2c1ccc(CC2CCN(CCCF)C2)cc1]

### Step 1: Methyl 9-(4-((1-(3-fluoropropyl)pyrrolidin-3-yl)methyl)phenyl)-6,7-dihydro-5H-benzo[7]annulene-3-carboxylate Isomer 1

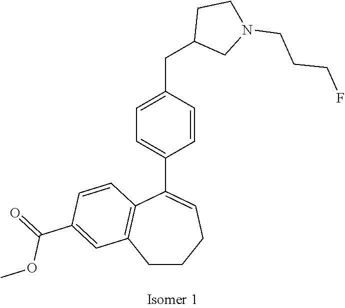 [SMILES: COC(=O)c1ccc2c(c1)CCCC=C2c1ccc(CC2CCN(CCCF)C2)cc1]

A mixture of methyl 9-(4,4,5,5-tetramethyl-1,3,2-dioxaborolan-2-yl)-6,7-dihydro-5H-benzo[7]annulene-3-carboxylate (Intermediate 2) (9.09 g, 27.7 mmol), 3-[(4-bromophenyl)methyl]-1-(3-fluoropropyl)pyrrolidine, Isomer 1 (Intermediate 1, Isomer 1) (7.55 g, 25.1 mmol) (15 g, 52.41 mmol), Pd(dppf)Cl₂ complex with DCM (1.23 g, 1.51 mmol), Cs₂CO₃ (20.49 g, 62.89 mmol) in dioxane (75 ml) and water (30 ml) was heated to reflux for 4 hours. After cooling to room temperature, DCM (80 ml) and water (80 ml) were added, and decantation was done by hydrophobic column. The organic phase was concentrated under reduced pressure and the residue obtained was purified by flash chromatography, eluting with a gradient of DCM/MeOH: from 100/00 to 95/05 to give 8.44 g (80%) of methyl 9-(4-((1-(3-fluoropropyl)pyrrolidin-3-yl)methyl)phenyl)-6,7-dihydro-5H-benzo[7]annulene-3-carboxylate Isomer 1 as an orange viscous oil.

LC/MS (m/z, MH+): 422

### Step 2: Methyl 8-bromo-9-(4-((1-(3-fluoropropyl)pyrrolidin-3-yl)methyl)phenyl)-6,7-dihydro-5H-benzo[7]annulene-3-carboxylate, Isomer 1

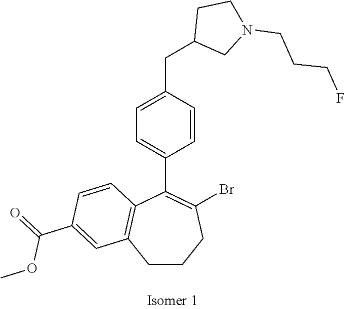 [SMILES: COC(=O)c1ccc2c(c1)CCCC(Br)=C2c1ccc(CC2CCN(CCCF)C2)cc1]

To a mixture of methyl 9-(4-((1-(3-fluoropropyl)pyrrolidin-3-yl)methyl)phenyl)-6,7-dihydro-5H-benzo[7]annulene-3-carboxylate Isomer 1 (8.58 g, 20.4 mmol) in DCM (90 ml) was added pyridinium tribromide (8.46 g, 26.5 mmol). The reaction mixture was stirred for 2 hours at room temperature. Water (100 ml) and DCM (150 ml) were added and pH was adjusted to 10 with concentrated solution of NaHCO₃ and K₂CO₃. The aqueous phase was washed 3 times with DCM and the gathered organic phases were dried over MgSO₄, filtered, concentrated under reduced pressure and the residue obtained was purified by flash chromatography eluting with a gradient of DCM/MeOH: from 100/00 to 95/05 to give 6.1 g (60%) of methyl 8-bromo-9-(4-((1-(3-fluoropropyl)pyrrolidin-3-yl)methyl)phenyl)-6,7-dihydro-5H-benzo[7]annulene-3-carboxylate, Isomer 1.

LC/MS (m/z, MH+): 500

### Intermediate 8: 3-[(4-Bromophenyl)methyl]-1-(3,3-difluoropropyl)pyrrolidine, Isomer 1

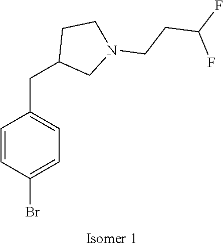 [SMILES: FC(F)CCN1CCC(Cc2ccc(Br)cc2)C1]

### Step 1: 3-[(4-Bromophenyl)methyl]pyrrolidine, 2,2,2-trifluoroacetic acid, Isomer 1

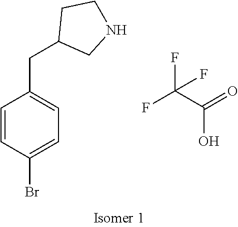 [SMILES: Brc1ccc(CC2CCNC2)cc1 ; O=C(O)C(F)(F)F]

To a solution of tert-butyl 3-[(4-bromophenyl)methyl]pyrrolidine-1-carboxylate Isomer 1 (2.11 g, 5.6 mmol) in DCM (50 ml) was added TFA (30 ml) and the mixture was stirred at RT for 4 hours. The solution was concentrated under vacuum and the residue obtained was treated with Et₂O. The solid formed was filtered and dried to give 1.98 g (90%) of 3-[(4-bromophenyl)methyl]pyrrolidine, 2,2,2-trifluoroacetic acid, Isomer 1.

LC/MS (m/z, MH+): 240

### Step 2: 3,3-Difluoropropyl 4-methylbenzenesulfonate

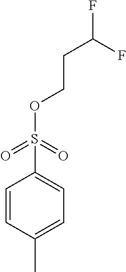 [SMILES: Cc1ccc(S(=O)(=O)OCCC(F)F)cc1]

To a solution of 3,3-difluoroprapan-1-ol (500 mg, 5.2 mmol) in THF (10 ml) cooled at 0° C. was added NaH (229 mg, 5.72 mmol, 60% purity) and the mixture was stirred at 0° C. for 1 hour. Then 4-methylbenzensulfonyl chloride (1.04 g, 5.46 mmol) was added. After 1 hour of stirring at RT, a concentrated solution of NH₄Cl (20 ml) was added and the mixture was extracted with EtOAc (3×50 ml). The combined organic phases were dried over Na₂SO₄, filtered, concentrated under reduced pressure and the residue obtained was purified by flash chromatography eluting with DCM to give 1.03 g (79%) of 3,3-difluoropropyl 4-methylbenzenesulfonate.

LC/MS (m/z, MH+): 251

### Step 3: 3-[(4-Bromophenyl)methyl]-1-(3,3-difluoropropyl)pyrrolidine, Isomer 1

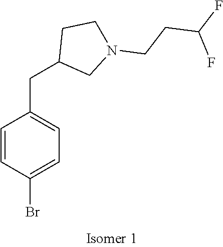 [SMILES: FC(F)CCN1CCC(Cc2ccc(Br)cc2)C1]

To a mixture of 3-[(4-bromophenyl)methyl]pyrrolidine, 2,2,2-trifluoroacetic acid Isomer 1 (1.2 g, 3.39 mmol) and K₂CO₃ in MeCN (30 ml) was added 3,3-difluoropropyl 4-methylbenzenesulfonate (1.02 g, 4.07 mmol) and the mixture was heated at 80° C. for 2 hours. The reaction mixture was quenched by addition of concentrated solution of NH₄Cl (50 ml) and extracted with EtOAc (3×50 ml). The combined organic phases were dried over Na₂SO₄, filtered, concentrated under reduced pressure and the residue obtained was purified by flash chromatography eluting with a gradient of DCM/EtOAc from 100/00 to 00/100 to give 607 mg (56%) of 3-[(4-bromophenyl)methyl]-1-(3,3-difluoropropyl)pyrrolidine, Isomer 1.

LC/MS (m/z, MH+): 318

### Intermediate 9: 1-(3-Fluoropropyl)-3-[[4-(4,4,5,5-tetramethyl-1,3,2-dioxaborolan-2-yl)phenyl]methyl]pyrrolidine, Isomer 1

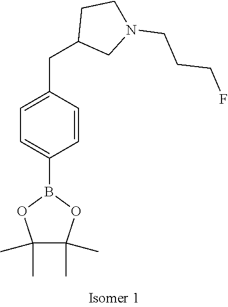 [SMILES: CC1(C)OB(c2ccc(CC3CCN(CCCF)C3)cc2)OC1(C)C]

A mixture of 3-[(4-bromophenyl)methyl]-1-(3-fluoropropyl)pyrrolidine, Isomer 1 (Intermediate 1 Isomer 1) (640 mg, 2.13 mmol), Pd(PPh₃)₂Cl₂ (109 mg, 0.15 mmol), bis(pinacolato)diboron (704 mg, 2.77 mmol) and AcOK (523 mg, 5.33 mmol) in dioxane (10 ml) was heated to reflux for 2 hours. After cooling, the reaction mixture was filtered on dicalite. The filtrate was concentrated under reduced pressure and the residue was purified by flash chromatography, eluting with a gradient of DCM/MeOH: from 100/00 to 90/10 to give 270 mg (36%) of 1-(3-fluoropropyl)-3-[[4-(4,4,5,5-tetramethyl-1,3,2-dioxaborolan-2-yl)phenyl]methyl]pyrrolidine, Isomer 1.

LC/MS (m/z, MH+): 348

### Intermediate 10: 1-(3,3-Difluoropropyl)-3-[[4-(4,4,5,5-tetramethyl-1,3,2-dioxaborolan-2-yl)phenyl]methyl]pyrrolidine, Isomer 1

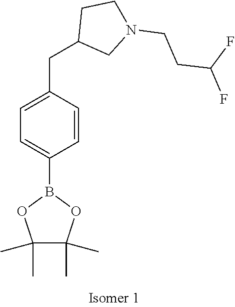 [SMILES: CC1(C)OB(c2ccc(CC3CCN(CCC(F)F)C3)cc2)OC1(C)C]

Intermediate 10 was prepared following a similar procedure to that of Intermediate 9 from 3-[(4-bromophenyl)methyl]-1-(3,3-difluoropropyl)pyrrolidine, Isomer 1 (Intermediate 8) to give 114.8 mg (87%) of 1-(3,3-difluoropropyl)-3-[[4-(4,4,5,5-tetramethyl-1,3,2-dioxaborolan-2-yl)phenyl]methyl]pyrrolidine, Isomer 1.

LC/MS (m/z, MH+): 366

### Intermediate 11: Methyl 8-bromo-9-(4-((1-(3,3-difluoropropyl)pyrrolidin-3-yl)methyl)phenyl)-6,7-dihydro-5H-benzo[7]annulene-3-carboxylate, Isomer 1

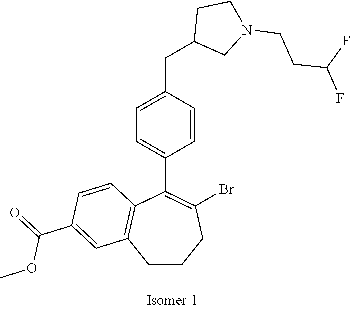 [SMILES: COC(=O)c1ccc2c(c1)CCCC(Br)=C2c1ccc(CC2CCN(CCC(F)F)C2)cc1]

### Step 1: Methyl 9-(4-((1-(3,3-difluoropropyl)pyrrolidin-3-yl)methyl)phenyl)-6,7-dihydro-5H-benzo[7]annulene-3-carboxylate, Isomer 1

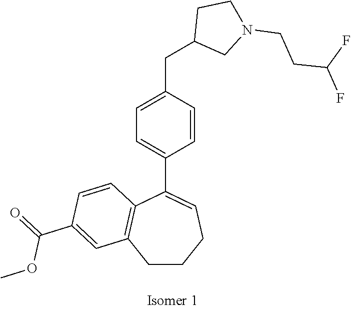 [SMILES: COC(=O)c1ccc2c(c1)CCCC=C2c1ccc(CC2CCN(CCC(F)F)C2)cc1]

Step 1 of Intermediate 11 was prepared following a similar procedure to that of Step 1 of Intermediate 7 from 3-[(4-bromophenyl)methyl]-1-(3,3-difluoropropyl)pyrrolidine, Isomer 1 (Intermediate 8) and methyl 9-(4,4,5,5-tetramethyl-1,3,2-dioxaborolan-2-yl)-6,7-dihydro-5H-benzo[7]annulene-3-carboxylate (Intermediate 2) to give 252 mg (59%) of methyl 8-bromo-9-(4-((1-(3,3-difluoropropyl)pyrrolidin-3-yl)methyl)phenyl)-6,7-dihydro-5H-benzo[7]annulene-3-carboxylate, Isomer 1.

LC/MS (m/z, MH+): 440

### Step 2: Methyl 8-bromo-9-(4-((1-(3,3-difluoropropyl)pyrrolidin-3-yl)methyl)phenyl)-6,7-dihydro-5H-benzo[7]annulene-3-carboxylate, Isomer 1

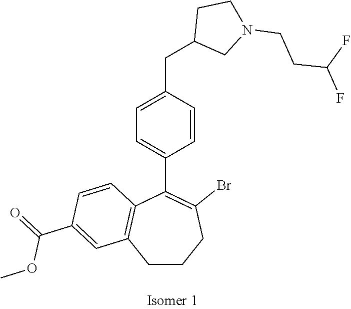 [SMILES: COC(=O)c1ccc2c(c1)CCCC(Br)=C2c1ccc(CC2CCN(CCC(F)F)C2)cc1]

Step 2 of Intermediate 11 was prepared following a similar procedure to that Step 2 of Intermediate 7 from methyl 8-bromo-9-(4-((1-(3,3-difluoropropyl)pyrrolidin-3-yl)methyl)phenyl)-6,7-dihydro-5H-benzo[7]annulene-3-carboxylate, Isomer 1 to give 212 mg (71%) of methyl 8-bromo-9-(4-((1-(3,3-difluoropropyl)pyrrolidin-3-yl)methyl)phenyl)-6,7-dihydro-5H-benzo[7]annulene-3-carboxylate, Isomer 1.

LC/MS (m/z, MH+): 518

### Intermediate 12: 8-(2,4-Dichlorophenyl)-9-(((trifluoromethyl)sulfonyl)oxy)-6,7-dihydro-5H-benzo[7]annulen-3-yl pivalate

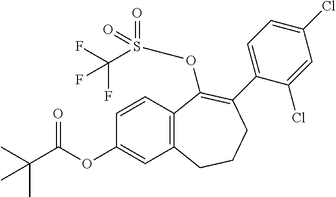 [SMILES: CC(C)(C)C(=O)Oc1ccc2c(c1)CCCC(c1ccc(Cl)cc1Cl)=C2OS(=O)(=O)C(F)(F)F]

### Step 1: 6-(2,4-Dichlorophenyl)-2-methoxy-6,7,8,9-tetrahydro-5H-benzo[7]annulen-5-one

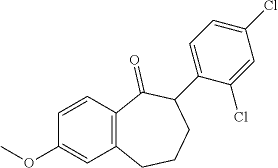 [SMILES: COc1ccc2c(c1)CCCC(c1ccc(Cl)cc1Cl)C2=O]

A mixture of 1-bromo-2,4-dichloro-benzene (890 mg, 3.94 mmol), 2-methoxy-6,7,8,9-tetrahydro-5H-benzo[7]annulen-5-one (500 mg, 2.63 mmol), Cs₂CO₃ (2.03 g, 10.5 mmol), 4,5-bis(diphenylphosphino)-9,9-dimethylxanthene (0.304 g, 0.53 mmol) and tris(dibenzylideneacetone)dipalladium(0) (0.3 g, 0.53 mmol) in xylene (10 ml) was heated to reflux for 16 hours. After cooling to room temperature, water (50 ml) and Et₂O (50 ml) were added. The organic phase was dried over MgSO₄, filtered and concentrated under reduced pressure. The residue obtained was purified by flash chromatography, eluting with a gradient of cyclohexane/EtOAc from 100/00 to 95/05 to give 470 mg (42%) of 6-(2,4-dichlorophenyl)-2-methoxy-6,7,8,9-tetrahydro-5H-benzo[7]annulen-5-one.

LC/MS (m/z, MH+): 335

### Step 2: 6-(2,4-Dichlorophenyl)-2-hydroxy-6,7,8,9-tetrahydro-5H-benzo[7]annulen-5-one

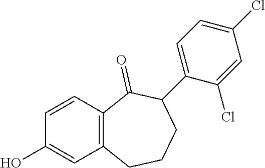 [SMILES: O=C1c2ccc(O)cc2CCCC1c1ccc(Cl)cc1Cl]

A mixture of 6-(2,4-dichlorophenyl)-2-methoxy-6,7,8,9-tetrahydro-5H-benzo[7]annulen-5-one (470 mg, 1.23 mmol) and aluminium chloride (395 mg, 2.96 mmol) in toluene (8 ml) was heated at 90° C. for 2 hours. After cooling to room temperature, water (20 ml) and EtOAc (50 ml) were added. The organic phase was separated, dried over MgSO₄, filtered and concentrated under reduced pressure. The residue obtained was purified by flash chromatography, eluting with a gradient of cyclohexane/EtOAc from 100/00 to 80/20 to give 165 mg (42%) of 6-(2,4-dichlorophenyl)-2-hydroxy-6,7,8,9-tetrahydro-5H-benzo[7]annulen-5-one.

LC/MS (m/z, MH+): 321

### Step 3: 6-(2,4-Dichlorophenyl)-5-oxo-6,7,8,9-tetrahydro-5H-benzo[7]annulen-2-yl pivalate

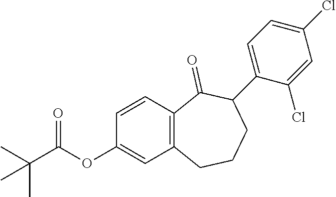 [SMILES: CC(C)(C)C(=O)Oc1ccc2c(c1)CCCC(c1ccc(Cl)cc1Cl)C2=O]

A mixture of 6-(2,4-dichlorophenyl)-2-hydroxy-6,7,8,9-tetrahydro-5H-benzo[7]annulen-5-one (163 mg, 0.51 mmol), potassium carbonate (77 mg, 0.56 mmol) and pivaloyl chloride (67 mg, 0.56 mmol) in acetone (5 ml) was heated at 40° C. for 1 hour. After cooling to room temperature, the reaction mixture was filtered. The filtrate was concentrated under reduced pressure and the residue obtained was purified by flash chromatography with a gradient of cyclohexane/EtOAc from 100/00 to 90/10 to give 205 mg (96%) of 6-(2,4-dichlorophenyl)-5-oxo-6,7,8,9-tetrahydro-5H-benzo[7]annulen-2-yl pivalate.

LC/MS (m/z, MH+): 405

### Step 4: 8-(2,4-Dichlorophenyl)-9-(((trifluoromethyl)sulfonyl)oxy)-6,7-dihydro-5H-benzo[7]annulen-3-yl pivalate

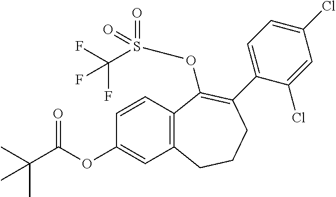 [SMILES: CC(C)(C)C(=O)Oc1ccc2c(c1)CCCC(c1ccc(Cl)cc1Cl)=C2OS(=O)(=O)C(F)(F)F]

To a solution of 6-(2,4-dichlorophenyl)-5-oxo-6,7,8,9-tetrahydro-5H-benzo[7]annulen-2-yl pivalate (197 mg, 0.48 mmol) in THE (5 ml) was added KHMDS (1 M, 0.58 ml, 0.58 mmol) at −50° C. under Ar atmosphere. The mixture was stirred for 30 min and 1,1,1-trifluoro-N-phenyl-N-(trifluoromethylsulfonyl)methanesulfonamide (173 mg, 0.49 mmol) was added to the resulting mixture. The reaction mixture was slowly warmed up to 20° C. and stirred for 90 minutes. The reaction mixture was quenched with saturated aqueous citric acid solution, then diluted with H₂O and extracted twice with Et₂O. The combined organic layers were dried over anhydrous Na₂SO₄, filtered, and concentrated under reduced pressure. The residue was purified by flash chromatography with a gradient of cyclohexane/EtOAc from 100/00 to 90/10 to give 160 mg (61%) of 8-(2,4-dichlorophenyl)-9-(((trifluoromethyl)sulfonyl)oxy)-6,7-dihydro-5H-benzo[7]annulen-3-yl pivalate.

LC/MS (m/z, MH+): 537

### Intermediate 13: Methyl 4-(2,4-dichlorophenyl)-5-(4,4,5,5-tetramethyl-1,3,2-dioxaborolan-2-yl)-2,3-dihydrobenzo[b]oxepine-8-carboxylate

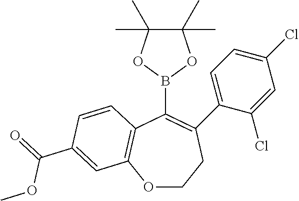 [SMILES: COC(=O)c1ccc2c(c1)OCCC(c1ccc(Cl)cc1Cl)=C2B1OC(C)(C)C(C)(C)O1]

### Step 1: 5-Oxo-2,3,4,5-tetrahydrobenzo[b]oxepin-8-yl trifluoromethanesulfonate

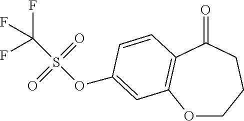 [SMILES: O=C1CCCOc2cc(OS(=O)(=O)C(F)(F)F)ccc21]

To a mixture of 8-hydroxy-3,4-dihydrobenzo[b]oxepin-5(2H)-one (4.2 g, 23.57 mmol) (prepared according to WO2018091153) and pyridine (2.82 g, 2.89 ml, 35.36 mmol) in DCM (120 ml) cooled at −20° C. was dropwise added trifluoromethanesulfonic anhydride (8.14 g, 6 ml, 28.28 mmol). The reaction mixture was stirred at 0° C. for 30 minutes. Water (50 ml) was added. The organic phase was separated and washed with an aqueous saturated solution of NaHCO₃ (50 ml). The organic phase was dried over MgSO₄, filtered, and concentrated under reduced pressure to give 7.30 g (100%) of 5-oxo-2,3,4,5-tetrahydrobenzo[b]oxepin-8-yl trifluoromethanesulfonate.

LC/MS (m/z, MH+): 311

### Step 2: Methyl 5-oxo-2,3,4,5-tetrahydrobenzo[b]oxepine-8-carboxylate

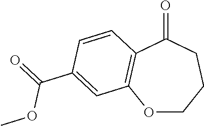 [SMILES: COC(=O)c1ccc2c(c1)OCCCC2=O]

To a solution of 5-oxo-2,3,4,5-tetrahydrobenzo[b]oxepin-8-yl trifluoromethanesulfonate (7.4 g, 23.85 mmol) in DMF (30 ml) and MeOH (15 ml) was added DIEA (3.15 g, 4.16 ml, 23.85 mmol) and Pd(dppf)Cl₂ complex with DCM (1.10 g, 1.43 mmol). The suspension was degassed and purged three times with CO. The mixture was stirred under CO (5 bars) at 75° C. for 2 hours. The reaction mixture was filtered through celite. The filtrate was diluted with water (400 ml) and extracted three times with EtOAc (300 ml). The combined organic phases were dried over Na₂SO₄, filtered, concentrated under reduced pressure and the residue obtained was purified by flash chromatography eluting with a gradient of heptane/ethyl acetate: from 85/15 to 80/20 to give 4.6 g (88%) methyl 5-oxo-2,3,4,5-tetrahydrobenzo[b]oxepine-8-carboxylate.

LC/MS (m/z, MH+): 221

### Step 3: Methyl 4-(2,4-dichlorophenyl)-5-oxo-2,3,4,5-tetrahydrobenzo[b]oxepine-8-carboxylate

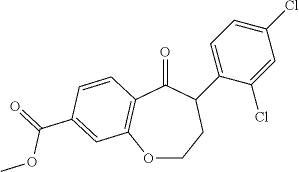 [SMILES: COC(=O)c1ccc2c(c1)OCCC(c1ccc(Cl)cc1Cl)C2=O]

Step 3 of Intermediate 13 was prepared following a similar procedure to that of step 1 of Intermediate 12 from methyl 5-oxo-2,3,4,5-tetrahydrobenzo[b]oxepine-8-carboxylate and 1-bromo-2,4-dichloro-benzene to give 1.13 g (67%) of methyl 4-(2,4-dichlorophenyl)-5-oxo-2,3,4,5-tetrahydrobenzo[b]oxepine-8-carboxylate.

LC/MS (m/z, MH+): 365

### Step 4: Methyl 4-(2,4-dichlorophenyl)-5-(((trifluoromethyl)sulfonyl)oxy)-2,3-dihydrobenzo[b]oxepine-8-carboxylate

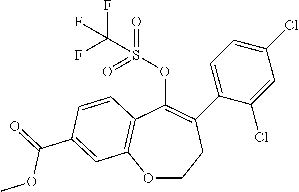 [SMILES: COC(=O)c1ccc2c(c1)OCCC(c1ccc(Cl)cc1Cl)=C2OS(=O)(=O)C(F)(F)F]

Step 4 of Intermediate 13 was prepared following a similar procedure to that of step 4 of Intermediate 12 from methyl 4-(2,4-dichlorophenyl)-5-oxo-2,3,4,5-tetrahydrobenzo[b]oxepine-8-carboxylate to give 0.48 g (31%) of methyl 4-(2,4-dichlorophenyl)-5-(((trifluoromethyl)sulfonyl)oxy)-2,3-dihydrobenzo[b]oxepine-8-carboxylate.

LC/MS (m/z, MH+): 496

### Step 5: Methyl 4-(2,4-dichlorophenyl)-5-(4,4,5,5-tetramethyl-1,3,2-dioxaborolan-2-yl)-2,3-dihydrobenzo[b]oxepine-8-carboxylate

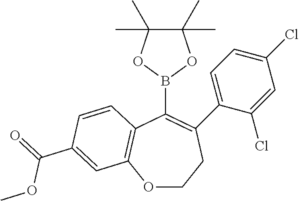 [SMILES: COC(=O)c1ccc2c(c1)OCCC(c1ccc(Cl)cc1Cl)=C2B1OC(C)(C)C(C)(C)O1]

Step 5 of Intermediate 13 was prepared following a similar procedure to that of Intermediate 2 from methyl 4-(2,4-dichlorophenyl)-5-(((trifluoromethyl)sulfonyl)oxy)-2,3-dihydrobenzo[b]oxepine-8-carboxylate to give 260 mg (61%) of methyl 4-(2,4-dichlorophenyl)-5-(4,4,5,5-tetramethyl-1,3,2-dioxaborolan-2-yl)-2,3-dihydrobenzo[b]oxepine-8-carboxylate.

LC/MS (m/z, MH+): 271

### Intermediate 14: Methyl 3-bromo-4-(4-((1-(3-fluoropropyl)pyrrolidin-3-yl)methyl)phenyl)-2H-thiochromene-7-carboxylate

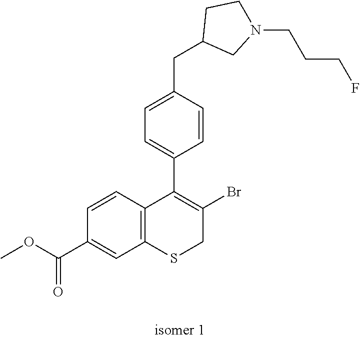 [SMILES: COC(=O)c1ccc2c(c1)SCC(Br)=C2c1ccc(CC2CCN(CCCF)C2)cc1]

### Step 1: 4-Oxothiochroman-7-yl trifluoromethanesulfonate

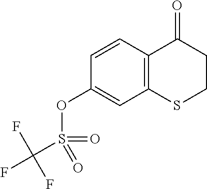 [SMILES: O=C1CCSc2cc(OS(=O)(=O)C(F)(F)F)ccc21]

Step 1 of Intermediate 14 was prepared following a similar procedure to that of step 1 of Intermediate 13 from 7-hydroxythiochroman-4-one (prepared according to WO2018091153) to give 1.14 g (33%) of 4-oxothiochroman-7-yl trifluoromethanesulfonate.

LC/MS (m/z, MH+): 313

### Step 2: Methyl 4-oxothiochromane-7-carboxylate

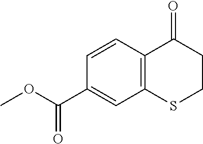

Step 2 of Intermediate 14 was prepared following a similar procedure to that of step 2 of Intermediate 13 from 4-oxothiochroman-7-yl trifluoromethanesulfonate to give 0.61 g (76%) of methyl 4-oxothiochromane-7-carboxylate.

LC/MS (m/z, MH+): 223

### Step 3: Methyl 4-(((trifluoromethyl)sulfonyl)oxy)-2H-thiochromene-7-carboxylate

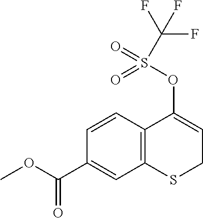 [SMILES: COC(=O)c1ccc2c(c1)SCC=C2OS(=O)(=O)C(F)(F)F]

Step 3 of Intermediate 14 was prepared following a similar procedure to that of step 4 of Intermediate 12 from methyl 4-oxothiochromane-7-carboxylate to give 199 mg (41%) of methyl 4-(((trifluoromethyl)sulfonyl)oxy)-2H-thiochromene-7-carboxylate.

LC/MS (m/z, MH+): 355

### Step 4: Methyl 4-(4,4,5,5-tetramethyl-1,3,2-dioxaborolan-2-yl)-2H-thiochromene-7-carboxylate

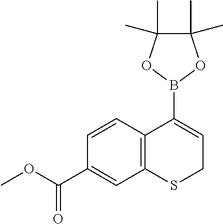

Step 4 of Intermediate 14 was prepared following a similar procedure to that of Intermediate 2 from methyl 4-(((trifluoromethyl)sulfonyl)oxy)-2H-thiochromene-7-carboxylate to give 1 g (35%) of methyl 4-(4,4,5,5-tetramethyl-1,3,2-dioxaborolan-2-yl)-2H-thiochromene-7-carboxylate.

LC/MS (m/z, MH+): 333

### Step 5: Methyl 4-(4-((1-(3-fluoropropyl)pyrrolidin-3-yl)methyl)phenyl)-2H-thiochromene-7-carboxylate

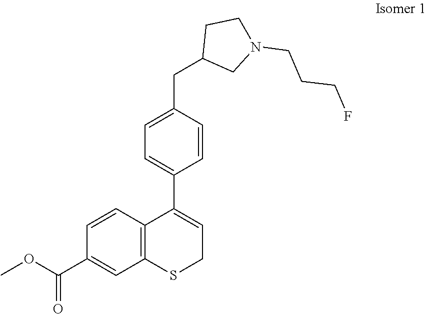 [SMILES: COC(=O)c1ccc2c(c1)SCC=C2c1ccc(CC2CCN(CCCF)C2)cc1]

Step 5 of Intermediate 14 was prepared following a similar procedure to that of step 1 of Intermediate 7 from methyl 4-(4,4,5,5-tetramethyl-1,3,2-dioxaborolan-2-yl)-2H-thiochromene-7-carboxylate and 3-[(4-bromophenyl)methyl]-1-(3-fluoropropyl)pyrrolidine, Isomer 1 (Intermediate 1, Isomer 1) to give 120 mg (47%) of methyl 4-(4-((1-(3-fluoropropyl)pyrrolidin-3-yl)methyl)phenyl)-2H-thiochromene-7-carboxylate, Isomer 1.

LC/MS (m/z, MH+): 426

### Step 6: Methyl 3-bromo-4-(4-((1-(3-fluoropropyl)pyrrolidin-3-yl)methyl)phenyl)-2H-thiochromene-7-carboxylate, Isomer 1

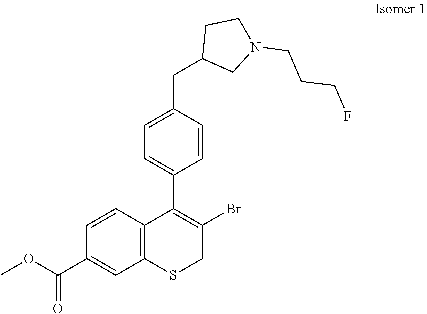 [SMILES: COC(=O)c1ccc2c(c1)SCC(Br)=C2c1ccc(CC2CCN(CCCF)C2)cc1]

Step 6 of Intermediate 14 was prepared following a similar procedure to that of step 2 of Intermediate 7 from methyl 4-(4-((1-(3-fluoropropyl)pyrrolidin-3-yl)methyl)phenyl)-2H-thiochromene-7-carboxylate to give 140 mg (64%) of methyl 3-bromo-4-(4-((1-(3-fluoropropyl)pyrrolidin-3-yl)methyl)phenyl)-2H-thiochromene-7-carboxylate, Isomer 1.

LC/MS (m/z, MH+): 504

### Intermediate 15: Methyl 6-(2,4-dichlorophenyl)-5-(trifluoromethylsulfonyloxy)-7,8-dihydronaphthalene-2-carboxylate

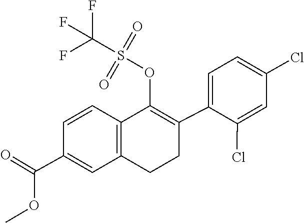 [SMILES: COC(=O)c1ccc2c(c1)CCC(c1ccc(Cl)cc1Cl)=C2OS(=O)(=O)C(F)(F)F]

### Step 1: Methyl 2-(2,4-dichlorophenyl)-1-oxo-tetralin-6-carboxylate

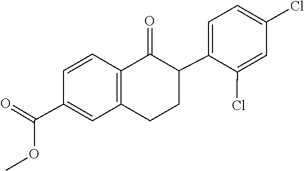 [SMILES: COC(=O)c1ccc2c(c1)CCC(c1ccc(Cl)cc1Cl)C2=O]

Step 1 of Intermediate 15 was prepared following a similar procedure to that of step 1 of Intermediate 12 from methyl 1-oxotetralin-6-carboxylate and 1-bromo-2,4-dichloro-benzene to give 459 mg (19%) of methyl 4-(2,4-dichlorophenyl)-5-oxo-2,3,4,5-tetrahydrobenzo[b]oxepine-8-carboxylate.

LC/MS (m/z, MH+): 349

### Step 2: Methyl 6-(2,4-dichlorophenyl)-5-(trifluoromethylsulfonyloxy)-7,8-dihydronaphthalene-2-carboxylate

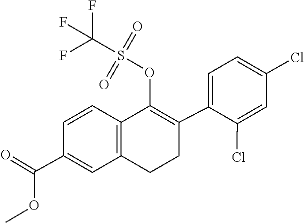 [SMILES: COC(=O)c1ccc2c(c1)CCC(c1ccc(Cl)cc1Cl)=C2OS(=O)(=O)C(F)(F)F]

Step 2 of Intermediate 15 was prepared following a similar procedure to that of step 2 of Intermediate 12 from methyl 2-(2,4-dichlorophenyl)-1-oxo-tetralin-6-carboxylate to give 0.48 g (31%) of methyl 6-(2,4-dichlorophenyl)-5-(trifluoromethylsulfonyloxy)-7,8-dihydronaphthalene-2-carboxylate.

LC/MS (m/z, MH+): 481

### Intermediate 16: Methyl 8-(2,4-difluorophenyl)-9-(4,4,5,5-tetramethyl-1,3,2-dioxaborolan-2-yl)-6,7-dihydro-5H-benzo[7]annulene-3-carboxylate

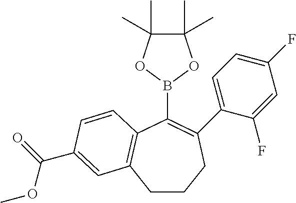 [SMILES: COC(=O)c1ccc2c(c1)CCCC(c1ccc(F)cc1F)=C2B1OC(C)(C)C(C)(C)O1]

### Step 1: Methyl 6-(2,4-difluorophenyl)-5-oxo-6,7,8,9-tetrahydro-5H-benzo[7]annulene-2-carboxylate

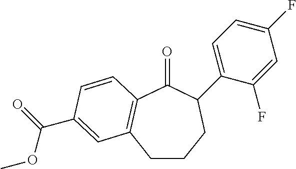 [SMILES: COC(=O)c1ccc2c(c1)CCCC(c1ccc(F)cc1F)C2=O]

Step 1 of Intermediate 16 was prepared following a similar procedure to that of step 1 of Intermediate 12 from methyl 5-oxo-6,7,8,9-tetrahydro-5H-benzo[7]annulene-2-carboxylate and 1-bromo-2,4-difluoro-benzene to give 2.55 g (34%) of methyl 6-(2,4-difluorophenyl)-5-oxo-6,7,8,9-tetrahydro-5H-benzo[7]annulene-2-carboxylate.

LC/MS (m/z, MH+): 331

### Step 2: Methyl 8-(2,4-difluorophenyl)-9-(((trifluoromethyl)sulfonyl)oxy)-6,7-dihydro-5H-benzo[7]annulene-3-carboxylate

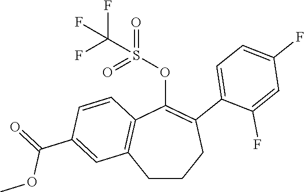 [SMILES: COC(=O)c1ccc2c(c1)CCCC(c1ccc(F)cc1F)=C2OS(=O)(=O)C(F)(F)F]

To a suspension of NaH (545 mg, 13.62 mmol, 60% purity in weight) in Me-THF (22 ml) cooled at 5° C. was added DBU (277 mg, 0.27 ml, 1.82 mmol) followed by a solution of methyl 6-(2,4-difluorophenyl)-5-oxo-6,7,8,9-tetrahydrobenzo[7]annulene-2-carboxylate (3 g, 9.08 mmol) and N,N-bis(trifluoromethylsulfonyl)aniline (4.2 g, 11.81 mmol) in THE (25 ml). The cooling bath was removed to allow the temperature to warm up to RT. A mixture of acetic acid (0.4 ml) and water (32 ml) was dropwise added, followed by water (100 ml) and EtOAc (150 ml). After decantation, the organic phase was dried over MgSO₄, filtered and concentrated under reduced pressure The residue obtained was purified by flash chromatography, eluting with DCM/heptane 50/50 to give 2.77 g (66%) of methyl 8-(2,4-difluorophenyl)-9-(((trifluoromethyl)sulfonyl)oxy)-6,7-dihydro-5H-benzo[7]annulene-3-carboxylate.

LC/MS (m/z, MH+): 463

### Step 3: Methyl 8-(2,4-difluorophenyl)-9-(4,4,5,5-tetramethyl-1,3,2-dioxaborolan-2-yl)-6,7-dihydro-5H-benzo[7]annulene-3-carboxylate

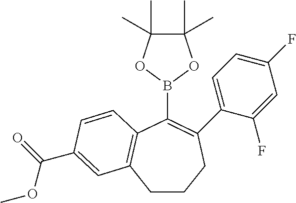 [SMILES: COC(=O)c1ccc2c(c1)CCCC(c1ccc(F)cc1F)=C2B1OC(C)(C)C(C)(C)O1]

Step 3 of Intermediate 16 was prepared following a similar procedure to that of Intermediate 2 from methyl 8-(2,4-difluorophenyl)-9-(((trifluoromethyl)sulfonyl)oxy)-6,7-dihydro-5H-benzo[7]annulene-3-carboxylate to give 1.89 g (72%) of methyl 8-(2,4-difluorophenyl)-9-(4,4,5,5-tetramethyl-1,3,2-dioxaborolan-2-yl)-6,7-dihydro-5H-benzo[7]annulene-3-carboxylate.

LC/MS (m/z, MH+): 441

### Intermediate 17: Methyl 8-(2-methyl-4-fluorophenyl)-9-(4,4,5,5-tetramethyl-1,3,2-dioxaborolan-2-yl)-6,7-dihydro-5H-benzo[7]annulene-3-carboxylate

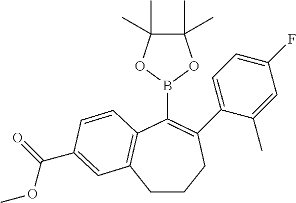 [SMILES: COC(=O)c1ccc2c(c1)CCCC(c1ccc(F)cc1C)=C2B1OC(C)(C)C(C)(C)O1]

### Step 1: Methyl 6-(2-methyl-4-fluorophenyl)-5-oxo-6,7,8,9-tetrahydro-5H-benzo[7]annulene-2-carboxylate

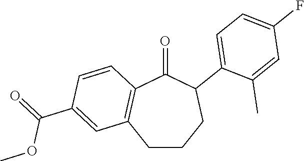 [SMILES: COC(=O)c1ccc2c(c1)CCCC(c1ccc(F)cc1C)C2=O]

Step 1 of Intermediate 17 was prepared following a similar procedure to that of step 1 of Intermediate 12 from 1-bromo-2-methyl-4-fluoro-benzene to give 700 mg (47%) of methyl 6-(2-methyl-4-fluorophenyl)-5-oxo-6,7,8,9-tetrahydro-5H-benzo[7]annulene-2-carboxylate.

LC/MS (m/z, MH+): 327

### Step 2: Methyl 8-(2-methyl-4-fluorophenyl)-9-(((trifluoromethyl)sulfonyl)oxy)-6,7-dihydro-5H-benzo[7]annulene-3-carboxylate

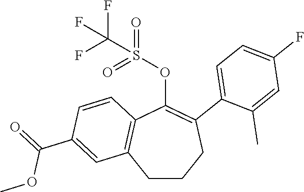 [SMILES: COC(=O)c1ccc2c(c1)CCCC(c1ccc(F)cc1C)=C2OS(=O)(=O)C(F)(F)F]

Step 2 of Intermediate 17 was prepared following a similar procedure to that of step 2 of Intermediate 16 from methyl 6-(2-methyl-4-fluorophenyl)-5-oxo-6,7,8,9-tetrahydrobenzo[7]annulene-2-carboxylate to give 7.86 g (82%) of methyl 8-(2-methyl-4-fluorophenyl)-9-(((trifluoromethyl)sulfonyl)oxy)-6,7-dihydro-5H-benzo[7]annulene-3-carboxylate.

LC/MS (m/z, MH+): 459

### Step 3: Methyl 8-(2-methyl-4-fluorophenyl)-9-(4,4,5,5-tetramethyl-1,3,2-dioxaborolan-2-yl)-6,7-dihydro-5H-benzo[7]annulene-3-carboxylate

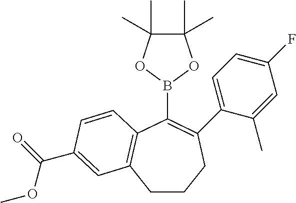 [SMILES: COC(=O)c1ccc2c(c1)CCCC(c1ccc(F)cc1C)=C2B1OC(C)(C)C(C)(C)O1]

Step 3 of Intermediate 17 was prepared following a similar procedure to that of Intermediate 2 from methyl 8-(2-methyl-4-fluorophenyl)-9-(((trifluoromethyl)sulfonyl)oxy)-6,7-dihydro-5H-benzo[7]annulene-3-carboxylate to give 3.93 g (43%) of methyl 8-(2-methyl-4-fluorophenyl)-9-(4,4,5,5-tetramethyl-1,3,2-dioxaborolan-2-yl)-6,7-dihydro-5H-benzo[7]annulene-3-carboxylate.

LC/MS (m/z, MH+): 437

### Intermediate 18: tert-Butyl 3-[[4-(4,4,5,5-tetramethyl-1,3,2-dioxaborolan-2-yl)phenyl]methyl]pyrrolidine-1-carboxylate, Isomer 1

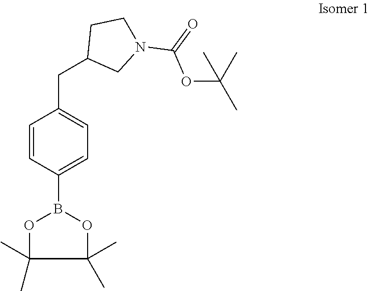 [SMILES: CC(C)(C)OC(=O)N1CCC(Cc2ccc(B3OC(C)(C)C(C)(C)O3)cc2)C1]

Intermediate 18 was prepared following a similar procedure to that of Intermediate 9 from tert-butyl 3-(4-bromobenzyl)pyrrolidine-1-carboxylate Isomer 1 (Step 1 of Intermediate 1) to give 2.53 g (74%) of tert-butyl 3-[[4-(4,4,5,5-tetramethyl-1,3,2-dioxaborolan-2-yl)phenyl]methyl]pyrrolidine-1-carboxylate, Isomer 1.

LC/MS (m/z, MH+): 388

### Intermediate 19: Methyl 8-(2-methyl-4-fluorophenyl)-9-(4,4,5,5-tetramethyl-1,3,2-dioxaborolan-2-yl)-6,7-dihydro-5H-benzo[7]annulene-3-carboxylate, Isomer 1

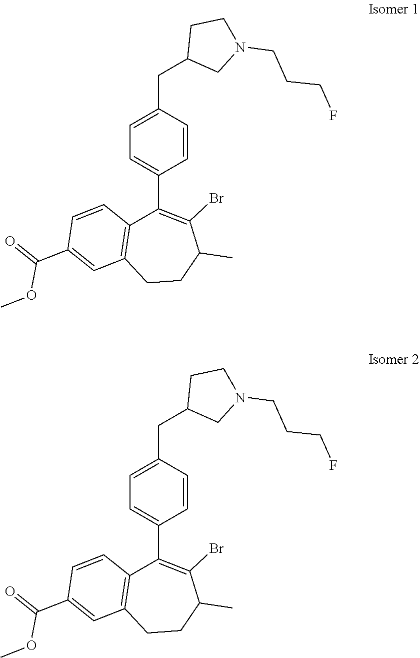 [SMILES: COC(=O)c1ccc2c(c1)CCC(C)C(Br)=C2c1ccc(CC2CCN(CCCF)C2)cc1 ; COC(=O)c1ccc2c(c1)CCC(C)C(Br)=C2c1ccc(CC2CCN(CCCF)C2)cc1]

### Step 1: Methyl 6-bromo-5-oxo-6,7,8,9-tetrahydro-5H-benzo[7]annulene-2-carboxylate

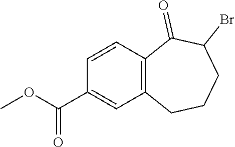 [SMILES: COC(=O)c1ccc2c(c1)CCCC(Br)C2=O]

To a mixture of methyl 5-oxo-6,7,8,9-tetrahydro-5H-benzo[7]annulene-2-carboxylate (9.42 g, 43.2 mmol) in DCM (400 mL) was portionwise added pyridinium tribromide (16.12 g, 45.4 mmol). The reaction mixture was stirred overnight at room temperature. Water (500 ml) and ether (1 L) were added. The organic phase was separated and washed twice with water, dried over MgSO₄, filtered and concentrated under reduced pressure to give 14.4 g (90%) of methyl 6-bromo-5-oxo-6,7,8,9-tetrahydro-5H-benzo[7]annulene-2-carboxylate.

LC/MS (m/z, MH+): 297

### Step 2: Methyl 5-oxo-8,9-dihydro-5H-benzo[7]annulene-2-carboxylate

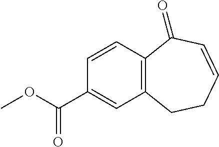 [SMILES: COC(=O)c1ccc2c(c1)CCC=CC2=O]

To a solution of methyl 6-bromo-5-oxo-6,7,8,9-tetrahydro-5H-benzo[7]annulene-2-carboxylate (10 g, 33.66 mmol) in acetonitrile (100 mL) was added DABCO (7.4 mL, 67.32 mmol). The reaction mixture was heated to 55° C. for 2.5 hours under Ar. Ether and 1N HCl were added. The organic phase was separated and washed twice with water, with brine, dried over MgSO₄, filtered and concentrated under reduced pressure. The resulting residue was then purified by flash chromatography eluting with a mixture of cyclohexane/EtOAc 85/15 to give 1.88 g (26%) of methyl 5-oxo-8,9-dihydro-5H-benzo[7]annulene-2-carboxylate.

LC/MS (m/z, MH+): 217

### Step 3: Methyl 7-methyl-5-oxo-6,7,8,9-tetrahydro-5H-benzo[7]annulene-2-carboxylate, racemic mixture

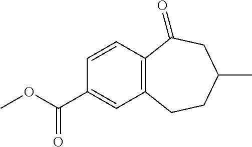

To a solution of methyl 5-oxo-8,9-dihydro-5H-benzo[7]annulene-2-carboxylate (1.88 g, 8.6 mmol) in THE (30 mL) under Ar at 0° C. was added a 0.328 M cuprate solution (35 mL, 11.5 mmol) prepared by addition of 15 mL of a 1.6 N solution of methyl lithium in ether to a suspension of 2.5 g of CuI (13 mmol) in 25 mL of ether under Ar at 0° C. The reaction mixture was stirred for 30 minutes at 0° C. Ether (200 mL) and 1N HCl (200 mL) were added. The organic phase was separated and the aqueous phase extracted with ether. The combined organic phases were washed with water, dried over MgSO₄, filtered and concentrated under reduced pressure. The resulting residue was then purified by flash chromatography eluting with a mixture of cyclohexane/EtOAc 95/5 to give 1.95 g (86%) of methyl 7-methyl-5-oxo-6,7,8,9-tetrahydro-5H-benzo[7]annulene-2-carboxylate as a racemic mixture.

LC/MS (m/z, MH+): 233

### Step 4: Methyl 7-methyl-9-(((trifluoromethyl)sulfonyl)oxy)-6,7-dihydro-5H-benzo[7]annulene-3-carboxylate, racemic mixture

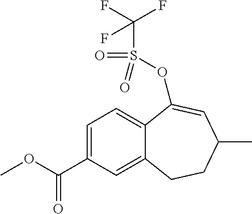

Step 4 of Intermediate 19 was prepared following a similar procedure to that of step 4 of Intermediate 12 from methyl 7-methyl-5-oxo-6,7,8,9-tetrahydro-5H-benzo[7]annulene-2-carboxylate to give 2.5 g (86%) of methyl 7-methyl-9-(((trifluoromethyl)sulfonyl)oxy)-6,7-dihydro-5H-benzo[7]annulene-3-carboxylate as a racemic mixture.

LC/MS (m/z, MH+): 365

### Step 5: Tert-butyl 3-(4-(3-(methoxycarbonyl)-7-methyl-6,7-dihydro-5H-benzo[7]annulen-9-yl)benzyl)pyrrolidine-1-carboxylate, Isomer 1

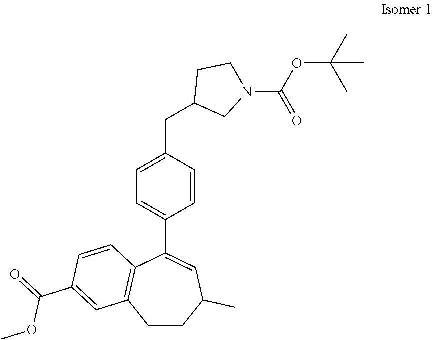 [SMILES: COC(=O)c1ccc2c(c1)CCC(C)C=C2c1ccc(CC2CCN(C(=O)OC(C)(C)C)C2)cc1]

Step 5 of Intermediate 19 was prepared following a similar procedure to that of step 1 of Intermediate 7 from methyl 7-methyl-9-(((trifluoromethyl)sulfonyl)oxy)-6,7-dihydro-5H-benzo[7]annulene-3-carboxylate and tert-Butyl 3-[[4-(4,4,5,5-tetramethyl-1,3,2-dioxaborolan-2-yl)phenyl]methyl]pyrrolidine-1-carboxylate, Isomer 1 (Intermediate 18) to give 1.02 g (76%) of tert-butyl 3-(4-(3-(methoxycarbonyl)-7-methyl-6,7-dihydro-5H-benzo[7]annulen-9-yl)benzyl)pyrrolidine-1-carboxylate, Isomer 1.

LC/MS (m/z, MH+): 476

### Step 6: Methyl 7-methyl-9-(4-(pyrrolidin-3-ylmethyl)phenyl)-6,7-dihydro-5H-benzo[7]annulene-3-carboxylate, 2,2,2-trifluoroacetic acid, Isomer 1

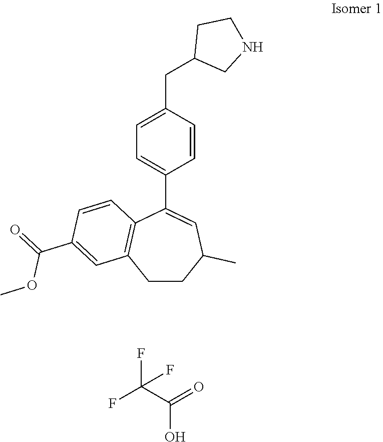 [SMILES: COC(=O)c1ccc2c(c1)CCC(C)C=C2c1ccc(CC2CCNC2)cc1 ; O=C(O)C(F)(F)F]

Step 6 of Intermediate 19 was prepared following a similar procedure to that of step 1 of Intermediate 8 from 3-(4-(3-(methoxycarbonyl)-7-methyl-6,7-dihydro-5H-benzo[7]annulen-9-yl)benzyl)pyrrolidine-1-carboxylate, Isomer 1 to give 753 mg (100%) of methyl 7-methyl-9-(4-(pyrrolidin-3-ylmethyl)phenyl)-6,7-dihydro-5H-benzo[7]annulene-3-carboxylate, 2,2,2-trifluoroacetic acid, Isomer 1.

LC/MS (m/z, MH+): 376

### Step 7: Methyl 9-(4-((1-(3-fluoropropyl)pyrrolidin-3-yl)methyl)phenyl)-7-methyl-6,7-dihydro-5H-benzo[7]annulene-3-carboxylate, Isomer 1

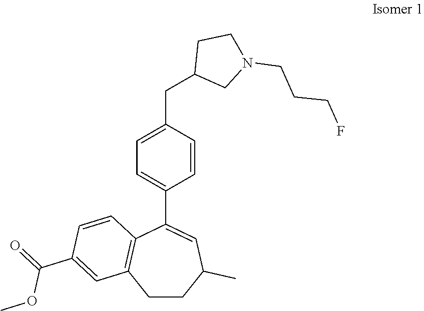 [SMILES: COC(=O)c1ccc2c(c1)CCC(C)C=C2c1ccc(CC2CCN(CCCF)C2)cc1]

To a solution of methyl 7-methyl-9-(4-(pyrrolidin-3-ylmethyl)phenyl)-6,7-dihydro-5H-benzo[7]annulene-3-carboxylate, 2,2,2-trifluoroacetic acid, Isomer 1 (753 mg, 1.538 mmol) in DCM (15 ml) was added NaOH (0.5 N, 7 ml) and, after 5 minutes, 1-fluoro-3-iodo-propane (0.166 ml, 1.692 mmol). The reaction mixture was stirred at RT overnight. After decantation, the organic phase was dried over Na₂SO₄, filtered, concentrated under reduced pressure and the residue obtained was purified by flash chromatography eluting with a gradient of DCM/MeOH from 100/00 to 90/10 to give 446 mg (67%) of methyl 9-(4-((1-(3-fluoropropyl)pyrrolidin-3-yl)methyl)phenyl)-7-methyl-6,7-dihydro-5H-benzo[7]annulene-3-carboxylate, Isomer 1.

LC/MS (m/z, MH+): 436

### Step 8: Methyl 8-bromo-9-(4-((1-(3-fluoropropyl)pyrrolidin-3-yl)methyl)phenyl)-7-methyl-6,7-dihydro-5H-benzo[7]annulene-3-carboxylate, Isomer 1

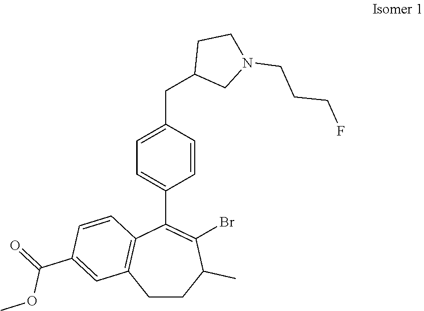 [SMILES: COC(=O)c1ccc2c(c1)CCC(C)C(Br)=C2c1ccc(CC2CCN(CCCF)C2)cc1]

Step 8 of Intermediate 19 was prepared following a similar procedure to that of step 2 of Intermediate 7 from methyl 9-(4-((1-(3-fluoropropyl)pyrrolidin-3-yl)methyl)phenyl)-7-methyl-6,7-dihydro-5H-benzo[7]annulene-3-carboxylate, Isomer 1 to give 372 mg (71%) of methyl 8-bromo-9-(4-((1-(3-fluoropropyl)pyrrolidin-3-yl)methyl)phenyl)-7-methyl-6,7-dihydro-5H-benzo[7]annulene-3-carboxylate, Isomer 1.

LC/MS (m/z, MH+): 514

### Intermediate 20: 4-Bromo-5-(4-((1-(3-fluoropropyl)pyrrolidin-3-yl)methyl)phenyl)-2,3-dihydrobenzo[b]thiepin-8-ol, Isomer 1

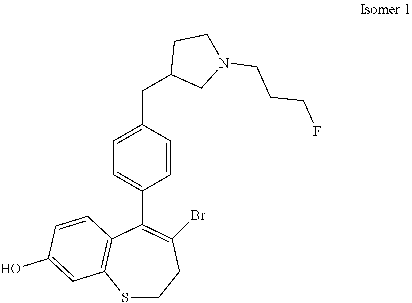 [SMILES: Oc1ccc2c(c1)SCCC(Br)=C2c1ccc(CC2CCN(CCCF)C2)cc1]

### Step 1: tert-Butyl 3-(4-(8-(pivaloyloxy)-2,3-dihydrobenzo[b]thiepin-5-yl)benzyl)pyrrolidine-1-carboxylate, Isomer 1

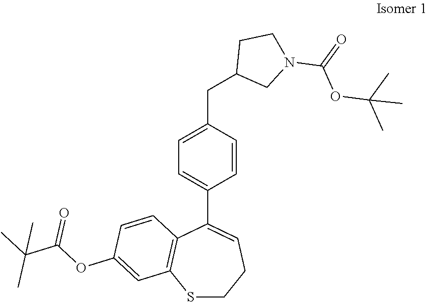 [SMILES: CC(C)(C)OC(=O)N1CCC(Cc2ccc(C3=CCCSc4cc(OC(=O)C(C)(C)C)ccc43)cc2)C1]

Step 1 of Intermediate 20 was prepared following a similar procedure to that of Step 1 of Intermediate 7 from 5-(((trifluoromethyl)sulfonyl)oxy)-2,3-dihydrobenzo[b]thiepin-8-yl pivalate (prepared according to WO2018091153) and tert-butyl 3-[[4-(4,4,5,5-tetramethyl-1,3,2-dioxaborolan-2-yl)phenyl]methyl]pyrrolidine-1-carboxylate, Isomer 1 (Intermediate 18) to give 810 mg (86%) of tert-butyl 3-(4-(8-(pivaloyloxy)-2,3-dihydrobenzo[b]thiepin-5-yl)benzyl)pyrrolidine-1-carboxylate, Isomer 1.

LC/MS (m/z, MH+): 522

### Step 2: 5-(4-(Pyrrolidin-3-ylmethyl)phenyl)-2,3-dihydrobenzo[b]thiepin-8-yl pivalate, 2,2,2-trifluoroacetic acid, Isomer 1

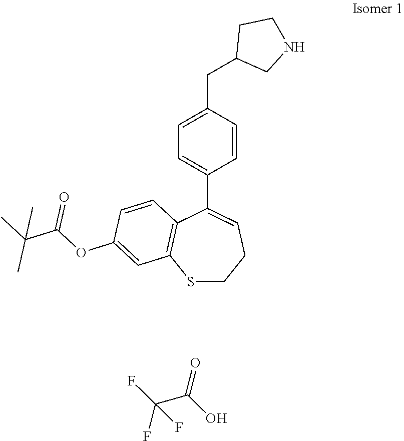 [SMILES: CC(C)(C)C(=O)Oc1ccc2c(c1)SCCC=C2c1ccc(CC2CCNC2)cc1 ; O=C(O)C(F)(F)F]

Step 2 of Intermediate 20 was prepared following a similar procedure to that of Step 1 of Intermediate 8 from tert-butyl 3-(4-(8-(pivaloyloxy)-2,3-dihydrobenzo[b]thiepin-5-yl)benzyl)pyrrolidine-1-carboxylate, Isomer 1 to give 1.1 g (crude) of 5-(4-(pyrrolidin-3-ylmethyl)phenyl)-2,3-dihydrobenzo[b]thiepin-8-yl pivalate, 2,2,2-trifluoroacetic acid, Isomer 1.

LC/MS (m/z, MH+): 422

### Step 3: 5-(4-((1-(3-Fluoropropyl)pyrrolidin-3-yl)methyl)phenyl)-2,3-dihydrobenzo[b]thiepin-8-yl pivalate, Isomer 1

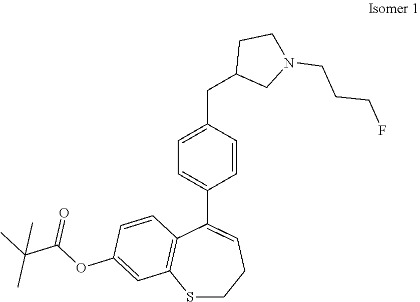 [SMILES: CC(C)(C)C(=O)Oc1ccc2c(c1)SCCC=C2c1ccc(CC2CCN(CCCF)C2)cc1]

Step 3 of Intermediate 20 was prepared following a similar procedure to that step 3 of Intermediate 1 from 5-(4-(pyrrolidin-3-ylmethyl)phenyl)-2,3-dihydrobenzo[b]thiepin-8-yl pivalate, 2,2,2-trifluoroacetic acid, Isomer 1 to give 490 mg (66%) of 5-(4-((1-(3-fluoropropyl)pyrrolidin-3-yl)methyl)phenyl)-2,3-dihydrobenzo[b]thiepin-8-yl pivalate, Isomer 1.

LC/MS (m/z, MH+): 482

### Step 4: 4-Bromo-5-(4-((1-(3-fluoropropyl)pyrrolidin-3-yl)methyl)phenyl)-2,3-dihydrobenzo[b]thiepin-8-yl pivalate, Isomer 1

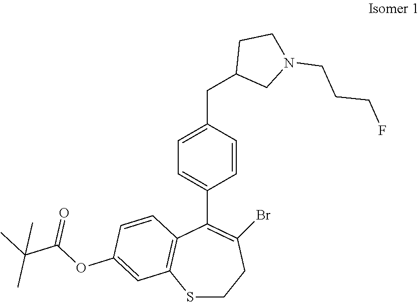 [SMILES: CC(C)(C)C(=O)Oc1ccc2c(c1)SCCC(Br)=C2c1ccc(CC2CCN(CCCF)C2)cc1]

Step 4 of Intermediate 20 was prepared following a similar procedure to that Step 2 of Intermediate 7 from 5-(4-((1-(3-fluoropropyl)pyrrolidin-3-yl)methyl)phenyl)-2,3-dihydrobenzo[b]thiepin-8-yl pivalate, Isomer 1 to give 360 mg (63%) of 4-bromo-5-(4-((1-(3-fluoropropyl)pyrrolidin-3-yl)methyl)phenyl)-2,3-dihydrobenzo[b]thiepin-8-yl pivalate, Isomer 1.

LC/MS (m/z, MH+): 560

### Step 5: 4-Bromo-5-(4-((1-(3-fluoropropyl)pyrrolidin-3-yl)methyl)phenyl)-2,3-dihydrobenzo[b]thiepin-8-ol, Isomer 1

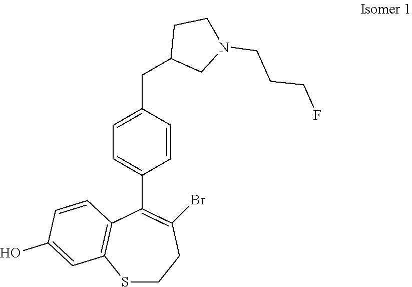 [SMILES: Oc1ccc2c(c1)SCCC(Br)=C2c1ccc(CC2CCN(CCCF)C2)cc1]

A mixture of 4-bromo-5-(4-((1-(3-fluoropropyl)pyrrolidin-3-yl)methyl)phenyl)-2,3-dihydrobenzo[b]thiepin-8-yl pivalate, Isomer 1 (360 mg, 0.64 mmol) in MeOH (7 ml) and NaOH 1N (2 ml, 2 mmol) was stirred at room temperature for 30 minutes. After addition of HCl 1N (2 ml), the reaction mixture was concentrated under reduced pressure. To the residue obtained, addition of DCM (60 ml) and water (20 ml). After decantation, the organic phase was dried over MgSO₄, filtered and concentrated under reduced pressure to give 360 mg (crude) of 4-bromo-5-(4-((1-(3-fluoropropyl)pyrrolidin-3-yl)methyl)phenyl)-2,3-dihydrobenzo[b]thiepin-8-ol, Isomer 1.

LC/MS (m/z, MH+): 476

### EXAMPLES

### Method A

### Example 1: 8-(2,4-Dichlorophenyl)-9-(4-((1-(3-fluoropropyl)pyrrolidin-3-yl)methyl)phenyl)-6,7-dihydro-5H-benzo[7]annulene-3-carboxylic acid, (S) Isomer

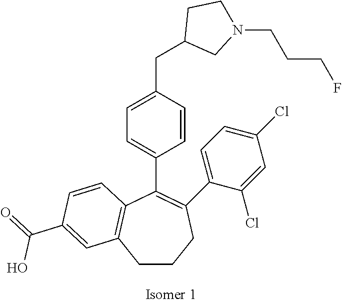 [SMILES: O=C(O)c1ccc2c(c1)CCCC(c1ccc(Cl)cc1Cl)=C2c1ccc(CC2CCN(CCCF)C2)cc1]

### Step 1: Methyl 8-(2,4-dichlorophenyl)-9-(4-((1-(3-fluoropropyl)pyrrolidin-3-yl)methyl)phenyl)-6,7-dihydro-5H-benzo[7]annulene-3-carboxylate, Isomer 1

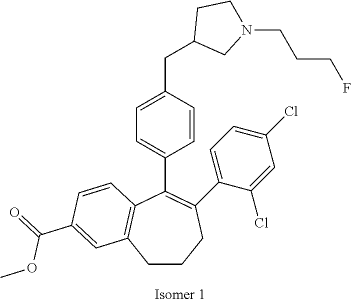 [SMILES: COC(=O)c1ccc2c(c1)CCCC(c1ccc(Cl)cc1Cl)=C2c1ccc(CC2CCN(CCCF)C2)cc1]

A mixture of methyl 8-(2,4-dichlorophenyl)-9-(4,4,5,5-tetramethyl-1,3,2-dioxaborolan-2-yl)-6,7-dihydro-5H-benzo[7]annulene-3-carboxylate (Intermediate 3) (101 mg, 214.75 μmol), 3-(4-bromobenzyl)-1-(3-fluoropropyl)pyrrolidine, Isomer 1 (Intermediate 1 Isomer 1) (65.8 mg, 219.14 μmol), Cs₂CO₃ (178.5 mg, 536.9 μmol), and Pd(dppf)Cl₂, complex with DCM (18 mg, 21 μmol) in dioxane (1 ml) and water (0.4 ml) was heated to reflux 1 hour. Water (2 ml) and DCM (5 ml) were added. After hydrophobic column decantation, the organic phase was concentrated under reduced pressure and the residue was treated on SCX column. The residue obtained was purified by flash chromatography eluting with a gradient of MeOH in DCM (100/0 to 95/05, v/v) to give 94 mg (77%) of methyl 8-(2,4-dichlorophenyl)-9-(4-((1-(3-fluoropropyl)pyrrolidin-3-yl)methyl)phenyl)-6,7-dihydro-5H-benzo[7]annulene-3-carboxylate, Isomer 1.

LC/MS (m/z, MH+): 566

### Step 2: 8-(2,4-Dichlorophenyl)-9-(4-((1-(3-fluoropropyl)pyrrolidin-3-yl)methyl)phenyl)-6,7-dihydro-5H-benzo[7]annulene-3-carboxylic acid, (S) Isomer

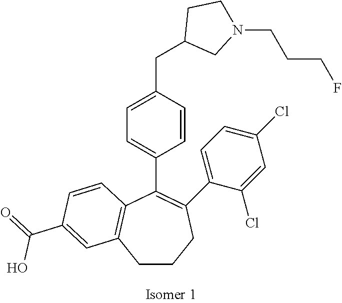 [SMILES: O=C(O)c1ccc2c(c1)CCCC(c1ccc(Cl)cc1Cl)=C2c1ccc(CC2CCN(CCCF)C2)cc1]

To a solution of methyl 8-(2,4-dichlorophenyl)-9-(4-((1-(3-fluoropropyl)pyrrolidin-3-yl)methyl)phenyl)-6,7-dihydro-5H-benzo[7]annulene-3-carboxylate, Isomer 1 (94 mg, 165.8 μmol) in MeOH (0.50 ml) and THF (0.5 ml) was added a solution of NaOH 1N (0.5 ml) and the reaction mixture was heated to reflux for 1 hour. After cooling, water (5 ml) and DCM (5 ml) were added and pH was adjusted to 2 with HCl 1N. After hydrophobic column decantation, the organic phase was concentrated under reduced pressure and the residue was purified by SFC (Flash DCPAK B 5 μm; 250×30 mm; supercritical CO₂ 75%/MeOH 25%/TEA 0.1% at 120 ml/min) to give 65.46 mg (71%) of 8-(2,4-dichlorophenyl)-9-(4-((1-(3-fluoropropyl)pyrrolidin-3-yl)methyl)phenyl)-6,7-dihydro-5H-benzo[7]annulene-3-carboxylic acid, (S) Isomer.

### Method B

### Example 2: 8-(2-Chloro-4-fluorophenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1

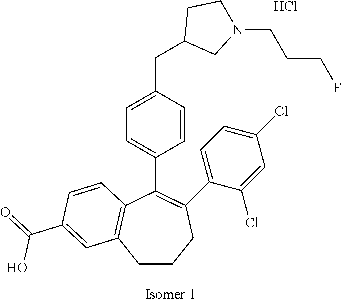 [SMILES: O=C(O)c1ccc2c(c1)CCCC(c1ccc(Cl)cc1Cl)=C2c1ccc(CC2CCN(CCCF)C2)cc1 ; Cl]

### Step 1: Methyl 8-(2-chloro-4-fluorophenyl)-9-(4-((1-(3-fluoropropyl)pyrrolidin-3-yl)methyl)phenyl)-6,7-dihydro-5H-benzo[7]annulene-3-carboxylate, Isomer 1

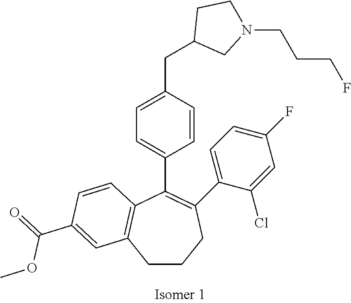 [SMILES: COC(=O)c1ccc2c(c1)CCCC(c1ccc(F)cc1Cl)=C2c1ccc(CC2CCN(CCCF)C2)cc1]

A mixture of methyl 8-bromo-9-(4-((1-(3-fluoropropyl)pyrrolidin-3-yl)methyl)phenyl)-6,7-dihydro-5H-benzo[7]annulene-3-carboxylate, Isomer 1 (Intermediate 7) (98 mg, 195.8 μmol), (2-chloro-4-fluoro-phenyl)boronic acid (35 mg, 203 μmol), Cs₂CO₃ (175 mg, 537 μmol), and Pd(dppf)Cl₂, complex with DCM (16 mg, 19 μmol) in dioxane (1 ml) and water (0.4 ml) was heated at 70° C. for 1 hour. After cooling to room temperature, Water (2 ml) and DCM (5 ml) were added. After hydrophobic column decantation, the organic phase was concentrated under reduced pressure and the residue was treated on SCX column to give 85 mg (79%) of methyl 8-(2-chloro-4-fluorophenyl)-9-(4-((1-(3-fluoropropyl)pyrrolidin-3-yl)methyl)phenyl)-6,7-dihydro-5H-benzo[7]annulene-3-carboxylate, Isomer 1.

LC/MS (m/z, MH+): 550

### Step 2: 8-(2-Chloro-4-fluorophenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1

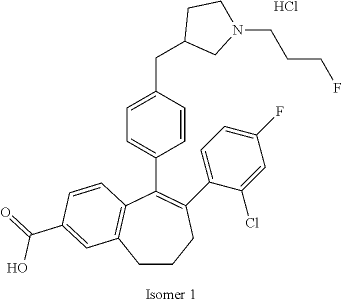 [SMILES: O=C(O)c1ccc2c(c1)CCCC(c1ccc(F)cc1Cl)=C2c1ccc(CC2CCN(CCCF)C2)cc1 ; Cl]

To a solution of methyl 8-(2-chloro-4-fluorophenyl)-9-(4-((1-(3-fluoropropyl)pyrrolidin-3-yl)methyl)phenyl)-6,7-dihydro-5H-benzo[7]annulene-3-carboxylate Isomer 1 (85 mg, 154.5 μmol) in MeOH (1 ml) and THF (1 ml) was added NaOH 1N (15 mg, 465 μmol) and the reaction mixture was heated to reflux for 40 minutes. After cooling, water (5 ml) and DCM (5 ml) were added and pH was adjusted to 2 with HCl 1N. After hydrophobic column decantation, the organic phase was concentrated under reduced pressure and the residue was purified by SFC (Flash DCPAK B 5 μm; 250×30 mm; supercritical CO₂ 70%/MeOH 30%/TEA 0.1% at 120 ml/min) to give 26.47 mg (30%) of 8-(2-chloro-4-fluorophenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1.

### Method C

### Example 36: 6-(2,4-Dichlorophenyl)-5-(4-((1-(3-fluoropropyl)pyrrolidin-3-yl)methyl)phenyl)-7,8-dihydronaphthalene-2-carboxylic acid hydrochloride, Isomer 1

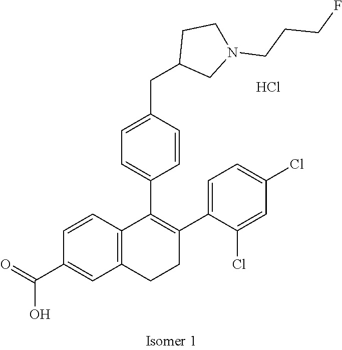 [SMILES: O=C(O)c1ccc2c(c1)CCC(c1ccc(Cl)cc1Cl)=C2c1ccc(CC2CCN(CCCF)C2)cc1 ; Cl]

### Step 1: Methyl 6-(2,4-dichlorophenyl)-5-(4-((1-(3-fluoropropyl)pyrrolidin-3-yl)methyl)phenyl)-7,8-dihydronaphthalene-2-carboxylate, Isomer 1

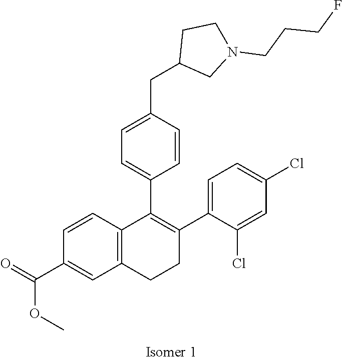 [SMILES: COC(=O)c1ccc2c(c1)CCC(c1ccc(Cl)cc1Cl)=C2c1ccc(CC2CCN(CCCF)C2)cc1]

A mixture of methyl 6-(2,4-dichlorophenyl)-5-(trifluoromethylsulfonyloxy)-7,8-dihydronaphthalene-2-carboxylate (Intermediate 15) (177 mg, 367 μmol), 1-(3-fluoropropyl)-3-[[4-(4,4,5,5-tetramethyl-1,3,2-dioxaborolan-2-yl)phenyl]methyl]pyrrolidine, Isomer 1 (Intermediate 9) (98 mg, 282 mol), Cs₂CO₃ (230 mg, 705 μmol), and Pd(dppf)Cl₂, complex with DCM (14 mg, 17 μmol) in dioxane (2 ml) and water (0.8 ml) was heated to 50° C. for 40 minutes. Water (2 ml) and DCM (4 ml) were added. After separation, the organic phase was concentrated under reduced pressure and the residue was purified by flash chromatography eluting with a solution of MeOH in DCM (95/05, v/v) to give 33 mg (21%) of methyl 6-(2,4-dichlorophenyl)-5-(4-((1-(3-fluoropropyl)pyrrolidin-3-yl)methyl)phenyl)-7,8-dihydronaphthalene-2-carboxylate, Isomer 1.

LC/MS (m/z, MH+): 552

### Step 2: 6-(2,4-Dichlorophenyl)-5-(4-((1-(3-fluoropropyl)pyrrolidin-3-yl)methyl)phenyl)-7,8-dihydronaphthalene-2-carboxylic acid hydrochloride, Isomer 1

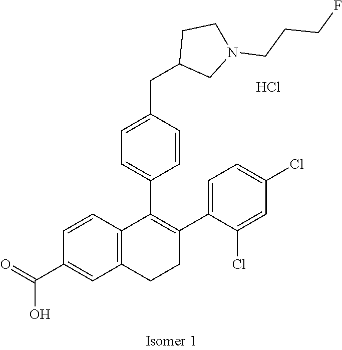 [SMILES: O=C(O)c1ccc2c(c1)CCC(c1ccc(Cl)cc1Cl)=C2c1ccc(CC2CCN(CCCF)C2)cc1 ; Cl]

Step 2 of Example 36 was prepared following a similar procedure to that of step 2 of Example 2 from methyl 6-(2,4-dichlorophenyl)-5-(4-((1-(3-fluoropropyl)pyrrolidin-3-yl)methyl)phenyl)-7,8-dihydronaphthalene-2-carboxylate Isomer 1 to give 34 mg (100%) of 6-(2,4-dichlorophenyl)-5-(4-((1-(3-fluoropropyl)pyrrolidin-3-yl)methyl)phenyl)-7,8-dihydronaphthalene-2-carboxylic acid hydrochloride, Isomer 1.

### Method D

### Example 3: 8-(4,4-Dimethylcyclohexyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1

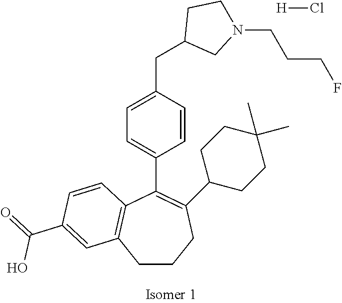

### Step 1: Methyl 8-(4,4-dimethylcyclohex-1-en-1-yl)-9-(4-((1-(3-fluoropropyl)pyrrolidin-3-yl)methyl)phenyl)-6,7-dihydro-5H-benzo[7]annulene-3-carboxylate, Isomer 1

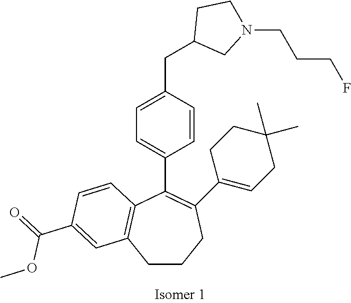 [SMILES: COC(=O)c1ccc2c(c1)CCCC(C1=CCC(C)(C)CC1)=C2c1ccc(CC2CCN(CCCF)C2)cc1]

Step 1 of Example 3 was prepared following a similar procedure to that of step 1 of Example 2 from 2-(4,4-dimethyl-1-cyclohexen-1-yl)-4,4,5,5-tetramethyl-1,3,2-dioxaborolane to give 99 mg (22%) of methyl 8-(4,4-dimethylcyclohex-1-en-1-yl)-9-(4-((1-(3-fluoropropyl)pyrrolidin-3-yl)methyl)phenyl)-6,7-dihydro-5H-benzo[7]annulene-3-carboxylate, Isomer 1.

LC/MS (m/z, MH+): 530.

### Step 2: Methyl 8-(4,4-dimethylcyclohexyl)-9-(4-((1-(3-fluoropropyl)pyrrolidin-3-yl)methyl)phenyl)-6,7-dihydro-5H-benzo[7]annulene-3-carboxylate, Isomer 1

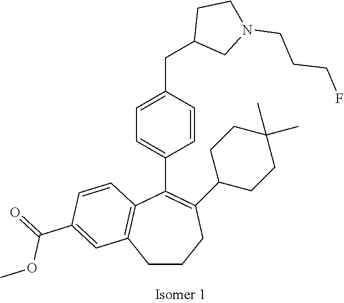 [SMILES: COC(=O)c1ccc2c(c1)CCCC(C1CCC(C)(C)CC1)=C2c1ccc(CC2CCN(CCCF)C2)cc1]

A mixture of methyl 8-(4,4-dimethylcyclohex-1-en-1-yl)-9-(4-((1-(3-fluoropropyl)pyrrolidin-3-yl)methyl)phenyl)-6,7-dihydro-5H-benzo[7]annulene-3-carboxylate, Isomer 1 (99 mg, 187 μmol), Pd/C 10% (21 mg, 200 μmol) in EtOH (6.5 ml) and EtOAc (4 ml) was hydrogenated at 50° C. and 5 bars of H₂ for 20 hours. The reaction mixture was filtered. The filtrate was evaporated under reduced pressure and the residue was purified by column chromatography (Amylose-1 5 μM; 250×30 mm; heptane/IsoPrOH/TEA 95/05/0.1) to give 35 mg (35%) of methyl 8-(4,4-dimethylcyclohexyl)-9-(4-((1-(3-fluoropropyl)pyrrolidin-3-yl)methyl)phenyl)-6,7-dihydro-5H-benzo[7]annulene-3-carboxylate, Isomer 1.

LC/MS (m/z, MH+): 532

### Step 3: 8-(4,4-Dimethylcyclohexyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1

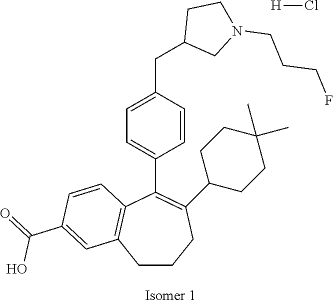

Step 3 of Example 3 was prepared following a similar procedure to that of step 2 of Example 2 from methyl 8-(4,4-dimethylcyclohexyl)-9-(4-((1-(3-fluoropropyl)pyrrolidin-3-yl)methyl)phenyl)-6,7-dihydro-5H-benzo[7]annulene-3-carboxylate, Isomer 1 to give 35 mg (97%) of 8-(4,4-dimethylcyclohexyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1.

### Method E

### Example 43: 4-(2,4-Dichlorophenyl)-5-(4-((1-(3-fluoropropyl)pyrrolidin-3-yl)methyl)phenyl)-2,3-dihydrobenzo[b]thiepin-8-ol, Isomer 1

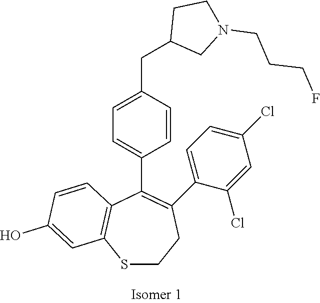 [SMILES: Oc1ccc2c(c1)SCCC(c1ccc(Cl)cc1Cl)=C2c1ccc(CC2CCN(CCCF)C2)cc1]

Example 43 was prepared following a similar procedure to that of Step 1 of Example 2 from 4-bromo-5-(4-((1-(3-fluoropropyl)pyrrolidin-3-yl)methyl)phenyl)-2,3-dihydrobenzo[b]thiepin-8-ol, Isomer 1 (Intermediate 20) and 2,4-dichlorophenylboronic acid to give 120 mg (70%) of 4-(2,4-dichlorophenyl)-5-(4-((1-(3-fluoropropyl)pyrrolidin-3-yl)methyl)phenyl)-2,3-dihydrobenzo[b]thiepin-8-ol, Isomer 1.

The compounds according to table 1 above were subjected to pharmacological tests for determining their degradation effects on estrogen receptors.

### Test: Estrogen Receptor Degradation Activity

Said test involves measuring the in vitro degradation activity of the compounds of the Table 1a.

The measurements of the degradation activities were made using a breast cancer cell ERα in cell western assay as described hereunder.

MCF7 cells (ATCC) were seeded in 384 wells microplate (collagen coated) at a concentration of 10000 cells/30 μL per well in red phenol free MEM alpha medium (invitrogen) containing 5% charcoal dextran striped FBS. The following day, 9 points serial 1:5 dilution of each compound was added to the cells in 2.5p L at final concentrations ranging from 0.3-0.0000018 μM (in table 2), or 0.1 μM for fulvestrant (using as positive control). At 4 hours post compound addition the cells were fixed by adding 25 μL of formalin (final concentration 5% formalin containing 0.1% triton) for 10 minutes at room temperature and then washed twice with PBS. Then, 50 μL of LI-COR blocking buffer containing 0.1% Triton was added to plate for 30 minutes at room temperature. LI-COR blocking buffer was removed and cells were incubated overnight at cold room with 50 μL anti-ER rabbit monoclonal antibody (Thermo scientific MA1-39540) diluted at 1:1000 in LI-COR blocking buffer containing 0.1% tween-20. Wells which were treated with blocking buffer but no antibody were used as background control. Wells were washed twice with PBS (0.1% tween-20) and incubated at 37° C. for 60 minutes in LI-COR (0.1% tween-20) containing goat anti-rabbit antibody Alexa 488 (1:1000) and Syto-64 a DNA dye (2 μM final concentration). Cells were then washed 3 times in PBS and scanned in ACUMEN explorer (TTP-Labtech). Integrated intensities in the green fluorescence and red fluorescence were measured to determine the levels of ERα and DNA respectively.

The degradation activity with respect to estrogen receptors in this test is given by the concentration which degrades 50% of the estrogen receptor (or IC₅₀) in nM.

The % of ERα levels decrease were determined as follows: % inhibition=100*(1−(sample−fulvestrant: DMSO−fulvestrant)).

The Table 2 below indicates the estrogen receptor degradation activity results for the compounds of Table 1a tested at 0.3 μM, and demonstrates that said compounds have a significant degradation activity on estrogen receptors.

TABLE 2

| | | % |
|---|---|---|
| | Degradation | Degradation |
| Compound No. | IC₅₀ (nM) | At 0.3 μM |
| 1 | 0.5 | 85 |
| 2 | 0.5 | 74 |
| 3 | 0.6 | 85 |
| 4 | 1.5 | 83 |
| 5 | 1.3 | 80 |
| 6 | 1.1 | 81 |
| 7 | 5.4 | 80 |
| 8 | 6.2 | 63 |
| 9 | 0.7 | 80 |
| 10 | 1.4 | 84 |
| 11 | 0.3 | 90 |
| 12 | 0.8 | 90 |
| 13 | 0.3 | 90 |
| 14 | 35 | 88 |
| 15 | 0.3 | 88 |
| 16 | 0.6 | 88 |
| 17 | 0.6 | 84 |
| 18 | 0.7 | 86 |
| 19 | 0.9 | 86 |
| 20 | 0.8 | 90 |
| 21 | 0.3 | 87 |
| 22 | 1.4 | 48 |
| 23 | 2.7 | 69 |
| 24 | 0.5 | 46 |
| 25 | 4.8 | 68 |
| 26 | 1.3 | 84 |
| 27 | 0.9 | 73 |
| 28 | 2.1 | 78 |
| 29 | 1.3 | 76 |
| 30 | 0.3 | 85 |
| 31 | 2.9 | 73 |
| 32 | 2.9 | 76 |
| 33 | 0.8 | 76 |
| 34 | 0.9 | 85 |
| 35 | 0.8 | 84 |
| 36 | 36.1 | 92 |
| 37 | 1.9 | 93 |
| 38 | 3 | 92 |
| 39 | 2 | 76 |
| 40 | 1.6 | 73 |
| 41 | 78.8 | 100 |
| 42 | 1.3 | 90 |
| 43 | 2.2 | 80 |
| 44 | 1.2 | 70 |

It is therefore apparent that the tested compounds have degradation activities for estrogen receptors, with IC50 less than 1 μM and with degradation levels greater than 40%, especially greater than 45% or 50%, and equal to or greater than 60% or 80% for most compounds. The compounds of formula (I) can therefore be used for preparing medicaments, especially medicaments which are degraders of estrogen receptors.

### Test: CYP3A4 Induction Potential for ADME Profile Evaluation

Said test evaluates the potential induction effect of the tested compounds on CYP3A4 gene expression in primary cultures of human hepatocytes from one or two donors with established concentration-dependent response to the reference inducer, rifampicin.

### Material and Methods

### Compounds and Chemicals:

Rifampicin and dimethylsulfoxide (DMSO) were purchased from Sigma-Aldrich (St. Louis, MO). All other chemicals and reagents used were obtained from usual commercial sources and were of the highest commercially available grade.

### Cell Media for Cryopreserved Human Hepatocytes:

The thawing (UCRM™) and plating (UPCM™) media were purchased from In Vitro ADMET Laboratories (Bethesda, MD). The culture medium was composed of Williams Medium E with 2 mM Glutamax, 0.1 μM dexamethasone, 1% ITS (10 μg/mL Insulin, 5.5 μg/mL Transferrin, 6.7 ng/mL Sodium Selenite, 50 μg/mL gentamycin (all purchased from Sigma-Aldrich).

### Thawing Procedures for Cryopreserved Human Hepatocytes:

Briefly, the vials containing the cryopreserved cells were removed from liquid nitrogen storage, thawed in a 37° C. water bath (75-90 seconds), and then quickly poured into prewarmed 50 mL UCRM™, centrifuged 10 min at 100 g, and re-diluted in UPCM™ (IVAL), so-called plating medium.

### Human Hepatocyte Cell Culture and Treatment:

Before seeding, cell viability was estimated using the Trypan blue dye exclusion test. Hepatocytes were seeded on collagen I-coated 96-well plates at a density of 70 000 cells/well in 100 μL of plating medium for at least 4 hours under a humidified atmosphere of CO₂/air (5%/95%), at 37° C. After the cell attachment period, the plating medium was removed, and the hepatocytes were treated daily for 2 consecutive days (approximately 48 hours), with 100 μL per well of fresh culture medium containing either the vehicle control (0.1% DMSO) or the investigated compounds at four concentrations 0.3, 1, 3 and 10 μM, or rifampicin, the reference inducer at 10 μM. All incubations were performed in duplicates.

Ten mM stock solutions of the different compounds were diluted to obtain 0.3, 1 and 3 mM daughter solutions. The day of the experiment, stock solutions were diluted 1000-fold in the incubation/culture medium to achieve final concentrations of 0.3, 1, 3 and 10 μM.

The quality of hepatocyte cultures prior to test compounds incubations was assessed by light microscopy. The morphology of the cultured cells, as well as the attachment efficiency and confluence, were observed and recorded daily. Only monolayers with minimum 80% confluency were used for further assessments.

### Assessment of Test Compounds Cytotoxicity:

Microscopical examination of hepatocyte morphology was used to evaluate cytotoxicity during incubation of each test compound. Moreover, to quantify the potential cytotoxic effects of the compounds in each experiment, a cytotoxicity assessment was also performed using the Resazurin Cell Viability Assay Kit (Abcam).

Induction Assay and Measurement of Gene expression:

After the 48-hour incubation period with the test compounds, the cell culture medium was removed, and the cells were lysed to release the RNA for CYP3A4 gene expression assessment. Quantification of CYP3A4 mRNA induction was performed by RT-PCR analysis.

Total RNA was extracted from hepatocytes using the King Fisher™ Flex Purification System with the MagMAX mirVana Total RNA Isolation kit (Thermo Fisher Scientific, USA). RNA was quantified by fluorimetry using the Quantifluor RNA System (Promega, USA) and cDNAs were synthesized from 0.1 g total RNA using the iScript Reverse Transcription Supermix for RT-qPCR from Biorad (France) at 42° C. for 30 min. cDNA samples were then diluted in water (1:5) and 5 μL of each sample was used for real-time PCR amplification by using the SYBR Green kit from Biorad (France). The following program was used: a denaturation step at 95° C. for 30 sec, and 40 cycles of PCR (denaturation, 95° C., 10 sec and annealing and extension, 58° C., 1 min). In all cases, the quality of the PCR product was assessed by monitoring a fusion step at the end of the run. Human forward and reverse primer sequences used were: 5′-CCT-GGC-ACC-CAG-CAC-AAT-3′ and 5′-GGG-CCG GAC-TCG-TCA-TAC-3′ for actin, 5′-CAC-AAA-CG-GAG-GCC-TTT-TG-3′ and 5′-ATC-CAT-GCT-GTA-GGC-CCC-AA-3′ for CYP3A4.

Cq (quantification cycle) values were determined using a multi-variable, non-linear regression model to individual well traces and then using this model an optimal Cq value was computed. Cq values are inversely proportional to the amount of target nucleic acid in the sample (i.e., lowest Cq level corresponds to the greatest amount of target nucleic acid in the sample). Cq of reference gene (in this study human actin) was also used as positive control of quantitative PCR.

### Calculation and Ratings:

The extent of induction or % of Eₘₐₓ, resulting from treatment with test compounds was calculated under the following formula:

$$\% E max = \frac{{E}_{compound} - {E}_{vehicle control}}{{E}_{positive control} - {E}_{vehicle control}} \times 100 \%$$

E_{Compound}: relative fold mRNA expression following treatment with test compound,
E_{Positive control}: relative fold mRNA expression following reference inducer i.e., 10 μM rifampicin,
E_{Vehicle control}: relative fold mRNA expression following vehicle treatment i.e., 0.1% DMSO,
E is the efficiency of the quantitative PCR.

The fold induction of test compounds over the level of vehicle control mRNA expression may also be determined and is calculated under the following formula:
Fold induction (FI) over vehicle control=E_{Compound}/E_{Vehicle control} with E_{Vehicle control} for mRNA expression set to 1.

The ratings to flag test compounds as inducer are based on % of Eₘₐₓ relative to rifampicin and are in the following table 3:

TABLE 3

| | |
|---|---|
| Eₘₐₓ ≤ 20% | Not inducer - Weak inducer (Low risk of |
| | DDI) |
| 20% < Eₘₐₓ < 45% | Intermediary inducer (Limited risk of DDI) |
| Eₘₐₓ ≥ 45% | Potentially high inducer (High risk of DDI) |

### Example A: Comparative Biological Activities of Compounds (C1) as Disclosed in WO2017/140669 and (C2) in the Scope of WO2018/091153

(C1) corresponds to the compound of example 51 of WO2017/140669 and (C2) corresponds to a compound in the scope of WO2018/091153, both compounds (C1) and (C2) being out of the scope of the present invention.

Compound (C1) has been synthetized according to example 51 of WO2017/140669. Compound (C1) displays an oxygen as a linker between north phenyl part and pyrrolidinyl part.

Compound (C2) is not specifically disclosed in WO2018/091153 but generally covered. It has been synthetized according to scheme 3a described in WO2018/091153. Compound (C2) displays a nitrogen as a linker between north phenyl part and pyrrolidinyl part.

### Example A.1: Preparation of Compound (C2)

### Compound (C2): (S)-8-(2,4-Dichlorophenyl)-9-(4-((1-(3-fluoropropyl)pyrrolidin-3-yl)amino)phenyl)-6,7-dihydro-5H-benzo[7]annulene-3-carboxylic acid

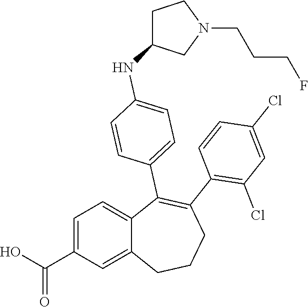 [SMILES: O=C(O)c1ccc2c(c1)CCCC(c1ccc(Cl)cc1Cl)=C2c1ccc(N[C@H]2CCN(CCCF)C2)cc1]

### Step 1: Methyl 9-(4-hydroxyphenyl)-6,7-dihydro-5H-benzo[7]annulene-3-carboxylate

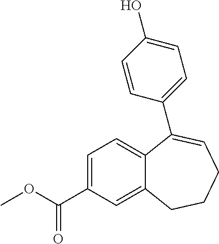 [SMILES: COC(=O)c1ccc2c(c1)CCCC=C2c1ccc(O)cc1]

To a mixture of methyl 9-(((trifluoromethyl)sulfonyl)oxy)-6,7-dihydro-5H-benzo[7]annulene-3-carboxylate (23.8 g, 67.94 mmol) (prepared according to WO2017140669), (4-hydroxyphenyl)boronic acid (10.85 g, 74.73 mmol) and [1,1′-bis(diphenylphosphino)ferrocene]dichloropalladium(II), complex with DCM (4.48 g, 6.11 mmol) in dioxane (200 ml) and water (70 ml) was added Cs₂CO₃ (44.32 g, 135.88 mmol). The reaction mixture was stirred for 30 minutes at RT. Water (500 ml) and EtOAc (500 ml) were added. The organic phase was dried over MgSO₄, filtered, and concentrated under reduced pressure. The residue was purified by flash chromatography eluting with a mixture of EtOAc and n-heptane (20/80; v/v) to give 17.36 g (87%) of methyl 9-(4-hydroxyphenyl)-6,7-dihydro-5H-benzo[7]annulene-3-carboxylate.

LC/MS (m/z, MH+): 295

### Step 2: Methyl 9-(4-(((trifluoromethyl)sulfonyl)oxy)phenyl)-6,7-dihydro-5H-benzo[7]annulene-3-carboxylate

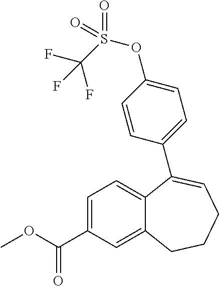 [SMILES: COC(=O)c1ccc2c(c1)CCCC=C2c1ccc(OS(=O)(=O)C(F)(F)F)cc1]

To a mixture of methyl 9-(4-hydroxyphenyl)-6,7-dihydro-5H-benzo[7]annulene-3-carboxylate (5.02 g, 17.1 mmol) in DCM (100 mL) cooled at 0° C. were added pyridine (2.5 mL, 30.91 mmol) and trifluoromethanesulfonic anhydride (8.55 g, 30.32 mmol). The reaction mixture was stirred at 0° C. for 30 minutes and RT for 1 hour. DCM (50 mL) and cold water (100 mL) were added, and decantation was done by hydrophobic column. The organic phase was concentrated under reduced pressure to give 7.2 g (99%) of methyl 9-(4-(((trifluoromethyl)sulfonyl)oxy)phenyl)-6,7-dihydro-5H-benzo[7]annulene-3-carboxylate which was used in the next step without further purification.

LC/MS (m/z, MH+): 427

### Step 3: Tert-butyl (S)-3-((4-(3-(methoxycarbonyl)-6,7-dihydro-5H-benzo[7]annulen-9-yl)phenyl)amino)pyrrolidine-1-carboxylate

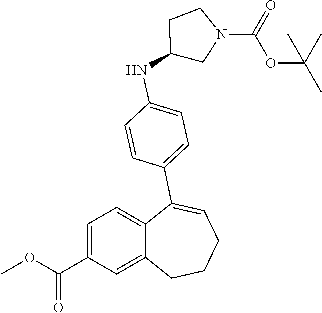 [SMILES: COC(=O)c1ccc2c(c1)CCCC=C2c1ccc(N[C@H]2CCN(C(=O)OC(C)(C)C)C2)cc1]

A mixture of methyl 9-(4-(((trifluoromethyl)sulfonyl)oxy)phenyl)-6,7-dihydro-5H-benzo[7]annulene-3-carboxylate (2.81 g, 6.59 mmol), tert-butyl (S)-3-aminopyrrolidine-1-carboxylate (2.45 g, 13.18 mmol), palladium(II) acetate (148 mg, 0.66 mmol), 4,5-bis(diphenylphosphino)-9,9-dimethylxanthene (0.53 g, 0.92 mmol) and Cs₂CO₃ (6.44 g, 19.77 mmol) in dioxane (27 mL) was heated to 140° C. for 2 hours under microwave irradiation. After cooling to RT, dichloromethane and water were added, and decantation was done by hydrophobic column. The organic phase was concentrated under reduced pressure. The residue was purified by flash chromatography, eluting with a gradient of heptane/EtOAc (from 100/00 to 70/30) to give 0.8 g (26%) of tert-butyl (S)-3-((4-(3-(methoxycarbonyl)-6,7-dihydro-5H-benzo[7]annulen-9-yl)phenyl)amino)pyrrolidine-1-carboxylate.

### Step 4: Methyl (S)-9-(4-(pyrrolidin-3-ylamino)phenyl)-6,7-dihydro-5H-benzo[7]annulene-3-carboxylate

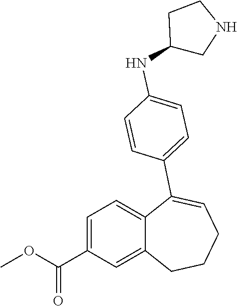 [SMILES: COC(=O)c1ccc2c(c1)CCCC=C2c1ccc(N[C@H]2CCNC2)cc1]

Step 4 of compound (C2) was prepared following a similar procedure to that of step 2 of Intermediate 1 Isomer 1 from tert-butyl (S)-3-((4-(3-(methoxycarbonyl)-6,7-dihydro-5H-benzo[7]annulen-9-yl)phenyl)amino)pyrrolidine-1-carboxylate to give 1.64 g (71%) of methyl (S)-9-(4-(pyrrolidin-3-ylamino)phenyl)-6,7-dihydro-5H-benzo[7]annulene-3-carboxylate.

LC/MS (m/z, MH+): 363

### Step 5: Methyl (S)-9-(4-((1-(3-fluoropropyl)pyrrolidin-3-yl)amino)phenyl)-6,7-dihydro-5H-benzo[7]annulene-3-carboxylate

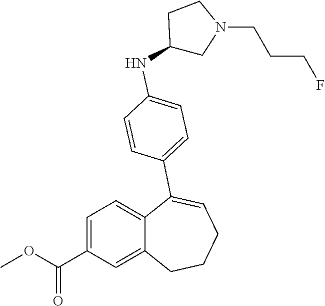 [SMILES: COC(=O)c1ccc2c(c1)CCCC=C2c1ccc(N[C@H]2CCN(CCCF)C2)cc1]

Step 5 of compound (C2) was prepared following a similar procedure to that of step 3 of Intermediate 1 Isomer 1 from methyl (S)-9-(4-(pyrrolidin-3-ylamino)phenyl)-6,7-dihydro-5H-benzo[7]annulene-3-carboxylate to give 2.17 g (100%) of methyl (S)-9-(4-((1-(3-fluoropropyl)pyrrolidin-3-yl)amino)phenyl)-6,7-dihydro-5H-benzo[7]annulene-3-carboxylate.

### Step 6: Methyl (S)-8-bromo-9-(4-((1-(3-fluoropropyl)pyrrolidin-3-yl)amino)phenyl)-6,7-dihydro-5H-benzo[7]annulene-3-carboxylate

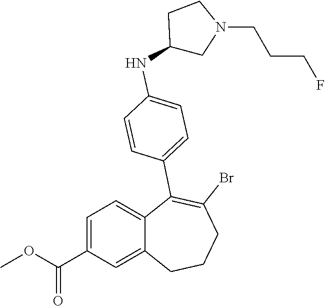 [SMILES: COC(=O)c1ccc2c(c1)CCCC(Br)=C2c1ccc(N[C@H]2CCN(CCCF)C2)cc1]

Step 6 of compound (C2) was prepared following a similar procedure to that Step 2 of Intermediate 7 from methyl (S)-9-(4-((1-(3-fluoropropyl)pyrrolidin-3-yl)amino)phenyl)-6,7-dihydro-5H-benzo[7]annulene-3-carboxylate to give 1.51 g (56%) of methyl (S)-8-bromo-9-(4-((1-(3-fluoropropyl)pyrrolidin-3-yl)amino)phenyl)-6,7-dihydro-5H-benzo[7]annulene-3-carboxylate.

LC/MS (m/z, MH+): 501

### Step 7: Methyl (S)-8-(2,4-dichlorophenyl)-9-(4-((1-(3-fluoropropyl)pyrrolidin-3-yl)amino)phenyl)-6,7-dihydro-5H-benzo[7]annulene-3-carboxylate

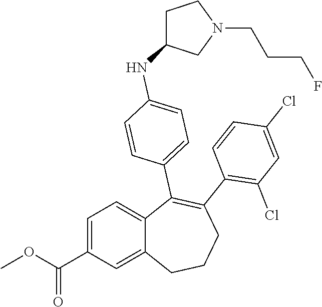 [SMILES: COC(=O)c1ccc2c(c1)CCCC(c1ccc(Cl)cc1Cl)=C2c1ccc(N[C@H]2CCN(CCCF)C2)cc1]

Step 7 was prepared following a similar procedure to that of Step 1 of Example 2 from methyl (S)-8-bromo-9-(4-((1-(3-fluoropropyl)pyrrolidin-3-yl)amino)phenyl)-6,7-dihydro-5H-benzo[7]annulene-3-carboxylate and 2,4-dichlorophenylboronic acid to give 182 mg (100%) of methyl (S)-8-(2,4-dichlorophenyl)-9-(4-((1-(3-fluoropropyl)pyrrolidin-3-yl)amino)phenyl)-6,7-dihydro-5H-benzo[7]annulene-3-carboxylate.

LC/MS (m/z, MH+): 567

### Step 8: (S)-8-(2,4-Dichlorophenyl)-9-(4-((1-(3-fluoropropyl)pyrrolidin-3-yl)amino)phenyl)-6,7-dihydro-5H-benzo[7]annulene-3-carboxylic acid (compound (C2))

Step 8 of compound (C2) was prepared following a similar procedure to that of step 2 of Example 1 from methyl (S)-8-(2,4-dichlorophenyl)-9-(4-((1-(3-fluoropropyl)pyrrolidin-3-yl)amino)phenyl)-6,7-dihydro-5H-benzo[7]annulene-3-carboxylate to give 71 mg (100%) of (S)-8-(2,4-dichlorophenyl)-9-(4-((1-(3-fluoropropyl)pyrrolidin-3-yl)amino)phenyl)-6,7-dihydro-5H-benzo[7]annulene-3-carboxylic acid.

LC/MS (m/z, MH+): 553.

1H NMR (400 MHz, DMSO-d6) δ ppm 1.44-1.58 (m, 1H), 1.70-1.90 (m, 2H), 2.03-2.22 (m, 5H), 2.29 (dd, J=9, 5 Hz, 1H), 2.39-2.62 (m, 5H), 2.76 (dd, J=9, 7 Hz, 1H), 2.84-2.94 (m, 2H), 3.68-3.86 (m, 1H), 4.47 (dt, J=47, 6 Hz, 2H), 5.75 (br d, J=6 Hz, 1H), 6.29 (d, J=9 Hz, 2H), 6.52 (d, J=9 Hz, 2H), 6.92 (d, J=8 Hz, 1H), 7.19 (d, J=8 Hz, 1H), 7.24-7.33 (m, 1H), 7.58 (d, J=2 Hz, 1H), 7.74 (dd, J=8, 1.8 Hz, 1H), 7.88 (d, J=2 Hz, 1H).

### Example A.2: Test: Estrogen Receptor Degradation Activity of Compound (C2)

Preliminary, the estrogen receptor degradation activity of the compound (C2) was tested following the same biological conditions as described above under “Test: Estrogen receptor degradation activity”.

TABLE 4

| | | % |
|---|---|---|
| | Degradation | Degradation |
| Compound No. | IC₅₀ (nM) | At 0.3 μM |
| C2 | 0.4 | 89 |

In Table 4, compound (C2), tested at 0.3 μM, presents a significant degradation activity on estrogen receptors.

In addition, as described in WO2017/140669, compound (C1) also displays a significant degradation activity on estrogen receptors (data not shown).

### Example A.3: Test: CYP 3A4 Induction Potential for ADME Profile Evaluation of the Compounds of Example 1, (C1) and (C2)

The Table 5 below shows comparative data regarding CYP3A4 induction of a compound of the present invention (example 1) with respect to compounds (C1) and (C2) under the test described above.

TABLE 5

| | | CYP3A4 |
|---|---|---|
| | | induction |
| Compound | | Emax |
| No. | Structure | (%) |
| Example 1 (invention) | 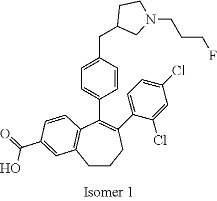 [SMILES: O=C(O)c1ccc2c(c1)CCCC(c1ccc(Cl)cc1Cl)=C2c1ccc(CC2CCN(CCCF)C2)cc1] | 7 |
| (C1) | 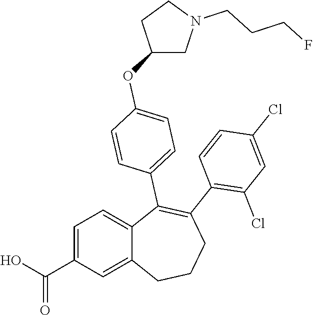 [SMILES: O=C(O)c1ccc2c(c1)CCCC(c1ccc(Cl)cc1Cl)=C2c1ccc(O[C@H]2CCN(CCCF)C2)cc1] | 93 |
| (C2) | 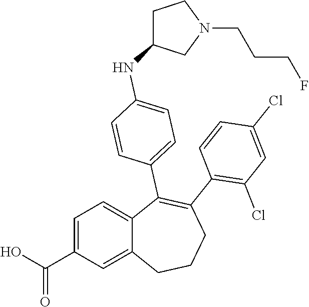 [SMILES: O=C(O)c1ccc2c(c1)CCCC(c1ccc(Cl)cc1Cl)=C2c1ccc(N[C@H]2CCN(CCCF)C2)cc1] | 85 |

Compounds of example 1, (C1) and (C2) are structurally close since they present same south part regarding the annulene ring and substituents, same north part with the fluoropropyl-pyrrolidine lateral chain, and differ only in the nature of the linkers between north phenyl part and pyrrolidinyl part. However, as shown in table 5, compound of example 1 of the invention demonstrates no CYP3A4 induction, in other terms has no CYP3A4 inductive effect, and thus displays an improved ADME profile compared to compounds (C1) and (C2). Hence, example 1 of the invention surprisingly displays an improved pharmacological profile, with a low risk of inducing drug-drug interactions, compared to prior art compounds (C1) and (C2). Such results were not predictable from prior art.

### Example B: CYP3A4 Induction Potential of Other Compounds of the Invention

Other compounds of the invention described above in table 1a were evaluated in the test “CYP3A4 induction potential”; results are presented in Table 6.

TABLE 6

| | CYP3A4 |
|---|---|
| | induction |
| Compound No. | Emax (%) |
| 2 | 23 |
| 3 | 8 |
| 4 | 30 |
| 5 | 13 |
| 6 | 8 |
| 11 | 11 |
| 12 | 34 |
| 13 | 26 |
| 16 | 22 |
| 21 | 42 |
| 30 | 15 |

The CYP3A4 induction potential results in Table 6 indicate that various compounds of the invention demonstrate no CYP3A4 induction, or a weak or intermediary CYP3A4 induction. Various examples of the invention thus display an improved pharmacological profile, with much lower risks of drug-drug interactions compared to compounds (C1) and (C2).

Accordingly, also provided herein are medicaments which comprise a compound of the formula (I), or a pharmaceutically acceptable salt thereof.

Herein are also provided the compounds of formula (I) defined above, or pharmaceutically acceptable salts thereof, for use as medicines.

Herein are also provided the compounds of formula (I) defined above, or pharmaceutically acceptable salt thereof, for use in therapy, especially as inhibitors and degraders of estrogen receptors, in particular displaying an improved pharmacological profile, typically a low or limited risk of drug-drug interactions, especially demonstrating no CYP3A4 induction, or a weak or intermediary CYP3A4 induction.

Herein are also provided the compounds of formula (I) defined above, or a pharmaceutically acceptable salts thereof, for use in the treatment of ovulatory dysfunction, cancer, endometriosis, osteoporosis, benign prostatic hypertrophy or inflammation.

A particular aspect is a compound of formula (I) defined above, or a pharmaceutically acceptable salt thereof, for use in the treatment of cancer.

In an embodiment, the cancer is a hormone dependent cancer.

In another embodiment, the cancer is an estrogen receptor dependent cancer, particularly the cancer is an estrogen receptor α dependent cancer.

In another embodiment, the cancer is selected from breast, ovarian, endometrial, prostate, uterine, cervical and lung cancer, or a metastasis thereof.

In another embodiment, the metastasis is a cerebral metastasis.

In another embodiment, the cancer is breast cancer. Particularly, the breast cancer is an estrogen receptor positive breast cancer (ERα positive breast cancer).

In another embodiment, the cancer is resistant to anti-hormonal treatment.

In a further embodiment, the compound of formula (I) is as used as single agent or in combination with other agents such as CDK4/6, mTOR or PI3K inhibitors.

According to another aspect, herein is provided a method of treating the pathological conditions indicated above, comprising administering to a subject in need thereof a therapeutically effective amount of a compound of formula (I), or a pharmaceutically acceptable salt thereof. In an embodiment of this method of treatment, the subject is a human.

Herein is also provided the use of a compound of the formula (I), or a pharmaceutically acceptable salt thereof, for the manufacture of a medicament useful in treating any of the pathological conditions indicated above, more particularly useful in treating cancer.

Herein are also provided the pharmaceutical compositions comprising as active principle a compound of formula (I). These pharmaceutical compositions comprise an effective dose of at least one compound of formula (I), or a pharmaceutically acceptable salt thereof, and also at least one pharmaceutically acceptable excipient.

The said excipients are selected, in accordance with the pharmaceutical form and method of administration desired, from the customary excipients, which are known to a person skilled in the art.

In the pharmaceutical compositions for oral, sublingual, subcutaneous, intramuscular, intravenous, topical, local, intra-tracheal, intranasal, transdermal or rectal administration, the active principle of formula (I) above, or its base, acid, zwitterion or salt thereof, may be administered in a unit administration form, in a mixture with conventional pharmaceutical excipients, to animals and to human beings for the treatment of the above disorders or diseases.

The unit administration forms appropriate include oral forms such as tablets, soft or hard gel capsules, powders, granules and oral solutions or suspensions, sublingual, buccal, intra-tracheal, intra-ocular and intra-nasal administration forms, forms for inhalative, topical, transdermal, subcutaneous, intra-muscular or intravenous administration, rectal administration forms and implants. For topical application it is possible to use the compounds of formula (I) in creams, gels, ointments or lotions.

As an example, a unit administration form of a compound of formula (I) in tablet form may comprise the following components:

| | | |
|---|---|---|
| Compound of formula (I) | 50.0 | mg |
| Mannitol | 223.75 | mg |
| Sodium croscarmellose | 6.0 | mg |
| Corn starch | 15.0 | mg |
| Hydroxypropylmethylcellulose | 2.25 | mg |
| Magnesium stearate | 3.0 | mg |

There may be particular cases in which higher or lower dosages are appropriate. According to usual practice, the dosage that is appropriate for each patient is determined by the doctor according to the mode of administration and the weight and response of the said patient.

1. A compound of the formula (I) or a pharmaceutically acceptable salt thereof: 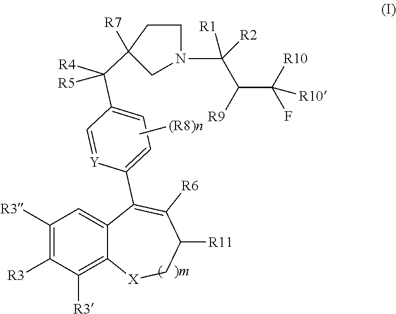
wherein
R1 and R2 independently represent a hydrogen atom or a deuterium atom;
R3 represents a hydrogen atom, a —COOH group or a —OH group;
R3′ and R3″ independently represent a hydrogen atom, a methyl group, a methoxy group, a chlorine atom, a fluorine atom, or a cyano group;
R4 and R5 independently represent a hydrogen atom, a fluorine atom, a —NH₂ group, a (C₁-C₃)alkyl group, a (C₁-C₃)alkoxy group, or a —OH group; or R4 and R5 together form an oxo group or R4 and R5 together form a ═NOCH₃ group or a (C₃-C₅)cycloalkyl group with the carbon atom to which they are attached;
R7 represents a hydrogen atom, a methyl group, a —OH group or a fluorine atom;
or alternatively R4 and R7 together form a cyclopropyl group together with the bond to which they are attached, that gives with the adjacent pyrrolidine group an azaspiro[2.4]heptane;
R6 represents a group selected from:
a phenyl group, said phenyl group being optionally substituted by 1 to 3 substituents independently selected from a halogen atom; a (C₁-C₆)alkyl group optionally substituted with a cyano group or a —OH group; a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group;
a fused phenyl group, selected from phenyl groups fused with a (C₃-C₆)cycloalkyl, which (C₃-C₆)cycloalkyl ring optionally comprises an unsaturation and wherein the fused phenyl is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
a bicyclic group comprising 5 to 12 carbon atoms, optionally comprising 1 to 2 unsaturations; optionally substituted with 1 to 4 substituents independently selected from: a fluorine atom, a —OH group, a (C₁-C₃)alkyl group, a (C₁-C₃)fluoroalkyl group, a (C₁-C₃)alkoxy group, a (C₁-C₃)fluoroalkoxy group and an oxo group;
a heteroaryl group comprising 2 to 9 carbon atoms and comprising from 1 to 3 heteroatoms independently selected from oxygen, nitrogen and sulfur, and at least 5 atoms including carbon atoms and heteroatoms, said heteroaryl group being optionally substituted with 1 to 3 substituents independently selected from a halogen atom, a (C₁-C₆)alkyl group, a (C₁-C₆)fluoroalkyl group, a (C₁-C₆)alkoxy group, a (C₁-C₆)fluoroalkoxy group, a cyano group, a carbamoyl group and a —OH group;
a cycloalkyl group comprising 3 to 7 carbon atoms, said cycloalkyl group being saturated or partially saturated and being optionally substituted with 1 to 4 substituents independently selected from:
a fluorine atom, a —OH group, a (C₁-C₃)alkyl group, a (C₁-C₃)fluoroalkyl group, a (C₁-C₃)alkoxy group, a (C₁-C₃)fluoroalkoxy group, an oxo group,
a (C₃-C₆)cycloalkyl group, and a phenyl group, said (C₃-C₆)cycloalkyl or phenyl groups being optionally substituted with one or two halogen atom(s) or (C₁-C₃)alkyl group(s);
a (C₃-C₆)cycloalkyl(C₁-C₃)alkyl group, optionally substituted on the cycloalkyl with 1 to 4 substituents independently selected from: a fluorine atom, a —OH group, a (C₁-C₄)alkyl group, a (C₁-C₃)fluoroalkyl group, a (C₁-C₃)fluoroalkoxy group and an oxo group;
a 4 to 7 membered-heterocycloalkyl group comprising 1 or 2 heteroatoms independently selected from oxygen, nitrogen and sulfur, said heterocycloalkyl group being saturated or partially saturated and being optionally substituted with 1 to 3 substituents independently selected from: a fluorine atom, a (C₁-C₃)alkyl group, a (C₁-C₃)fluoroalkyl group, a (C₁-C₃)fluoroalkoxy group, an oxo group, a (C₁-C₃)alkoxy group and a —OH group;
a (C₁-C₆)alkyl group, said alkyl group being optionally substituted with 1 to 4 substituents independently selected from: a fluorine atom, a (C₁-C₃)alkoxy group, a (C₁-C₃)fluoroalkoxy group and a —OH group; and
a phenyl(C₁-C₂)alkyl group, said phenyl group being optionally substituted with 1 to 3 substituents independently selected from a halogen atom; a (C₁-C₃)alkyl group; a (C₁-C₃)fluoroalkyl group; a (C₁-C₃)alkoxy group; a (C₁-C₃)fluoroalkoxy group; a cyano group; and a —OH group;
X represents —CH₂—, —O—, or —S—;
Y represents —CH═, —N═ or —CR″═, wherein R″ represents a (C₁-C₃)alkyl group or a halogen atom, a cyano group, or a (C₁-C₃)fluoroalkyl group;
R8 independently represents a (C₁-C₃)alkyl group a halogen atom, a cyano group, or a (C₁-C₃)fluoroalkyl group;
R9 represents a hydrogen atom or a fluorine atom;
R10 and R10′ independently represent a hydrogen atom or a fluorine atom;
R11 represents a hydrogen atom, or a (C₁-C₃)alkyl group or a cyclopropyl;
n is 0, 1 or 2, and
m is 0 or 1.

2. The compound of formula (I) according to claim 1, or a pharmaceutically acceptable salt thereof, characterized in that R1 and R2 are a hydrogen atom.

3. The compound of formula (I) according to claim 1, or a pharmaceutically acceptable salt thereof, characterized in that R3 is a —COOH group or a —OH group.

4. The compound of formula (I) according to claim 1, or a pharmaceutically acceptable salt thereof, characterized in that when m is 1 then X represents —CH₂— or —O—.

5. The compound of formula (I) according to claim 1, or a pharmaceutically acceptable salt thereof, characterized in that X represents —CH₂— or —O—.

6. The compound of formula (I) according to claim 1, or a pharmaceutically acceptable salt thereof, characterized in that R4 and R5 independently represent a hydrogen atom or a fluorine atom.

7. The compound of formula (I) according to claim 1, or a pharmaceutically acceptable salt thereof, characterized in that R7 represents a hydrogen atom or a fluorine atom.

8. The compound of formula (I) according to claim 1, or a pharmaceutically acceptable salt thereof, wherein:
R4 and R5 independently represent a hydrogen atom, a —NH₂ group, a (C₁-C₃)alkyl group, a (C₁-C₃)alkoxy group, or a —OH group; or R4 and R5 together form an oxo group or R4 and R5 together form a ═NOCH₃ group or a (C₃-C₅)cycloalkyl group with the carbon atom to which they are attached, and
R7 represents a hydrogen atom, a methyl group or a —OH group;
or alternatively R4 and R7 together form a cyclopropyl group together with the bond to which they are attached, that gives with the adjacent pyrrolidine group an azaspiro[2.4]heptane.

9. The compound of formula (I) according to claim 1, or a pharmaceutically acceptable salt thereof, characterized in that R6 represents a phenyl group, said phenyl group being optionally substituted with 1 to 3 substituents independently selected from a halogen atom; a (C₁-C₆)alkyl group and a (C₁-C₆)fluoroalkyl group.

10. The compound of formula (I) according to claim 1, or a pharmaceutically acceptable salt thereof, characterized in that R6 represents a pyridyl group, said pyridyl group being optionally substituted with 1 to 3 substituents independently selected from a (C₁-C₆)alkoxy group.

11. The compound of formula (I) according to claim 1, or a pharmaceutically acceptable salt thereof, characterized in that R6 represents a saturated or partially saturated cyclohexyl group, said saturated or partially saturated cyclohexyl group being optionally substituted with 1 to 4 substituents independently selected from a (C₁-C₃)alkyl group.

12. The compound of formula (I) according to claim 1, or a pharmaceutically acceptable salt thereof, characterized in that R3′ and R3″ both represent a hydrogen atom.

13. The compound of formula (I) according to claim 1, or a pharmaceutically acceptable salt thereof, characterized in that n is 0.

14. The compound of formula (I) according to claim 1, or a pharmaceutically acceptable salt thereof, wherein Y represents —CH═.

15. The compound of formula (I) according to claim 1, or a pharmaceutically acceptable salt thereof, wherein R9 represents a hydrogen atom.

16. The compound of formula (I) according to claim 1, or a pharmaceutically acceptable salt thereof, wherein R10 and R10′ both represent a hydrogen atom; or one of R10 and R10′ represents a hydrogen atom and the other a fluorine atom.

17. The compound of formula (I) according to claim 1, or a pharmaceutically acceptable salt thereof, wherein R11 represents a hydrogen atom or a methyl group.

18. The compound of formula (I) according to claim 1, or a pharmaceutically acceptable salt thereof, wherein m is 1.

19. The compound of formula (I) according to claim 1, or a pharmaceutically acceptable salt thereof, characterized in that said compound of formula (I) is selected from the following compounds:
8-(2,4-dichlorophenyl)-9-(4-((1-(3-fluoropropyl)pyrrolidin-3-yl)methyl)phenyl)-6,7-dihydro-5H-benzo[7]annulene-3-carboxylic acid, (S) Isomer (1),
8-(2-chloro-4-fluorophenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (2),
8-(4,4-dimethylcyclohexyl)-9-(4-((1-(3-fluoropropyl)pyrrolidin-3-yl)methyl)phenyl)-6,7-dihydro-5H-benzo[7]annulene-3-carboxylic acid, Isomer 1 (3),
9-(4-((1-(3,3-difluoropropyl)pyrrolidin-3-yl)methyl)phenyl)-8-(4-fluoro-2-methylphenyl)-6,7-dihydro-5H-benzo[7]annulene-3-carboxylic acid hydrochloride, Isomer 1 (4),
9-(4-((1-(3,3-difluoropropyl)pyrrolidin-3-yl)methyl)phenyl)-8-(4-fluoro-2-(trifluoromethyl)phenyl)-6,7-dihydro-5H-benzo[7]annulene-3-carboxylic acid hydrochloride, Isomer 1 (5),
8-(3-chloro-2-methylphenyl)-9-(4-((1-(3,3-difluoropropyl)pyrrolidin-3-yl)methyl)phenyl)-6,7-dihydro-5H-benzo[7]annulene-3-carboxylic acid hydrochloride, Isomer 1 (6),
4-(2,4-dichlorophenyl)-5-(4-((1-(3-fluoropropyl)pyrrolidin-3-yl)methyl)phenyl)-2,3-dihydrobenzo[b]oxepine-8-carboxylic acid hydrochloride, Isomer 1 (7),
4-(2,4-dichlorophenyl)-5-(4-((1-(3-fluoropropyl)pyrrolidin-3-yl)methyl)phenyl)-2,3-dihydrobenzo[b]oxepine-8-carboxylic acid hydrochloride, Isomer 2 (8),
8-(2,4-dichlorophenyl)-9-(4-((1-(3,3-difluoropropyl)pyrrolidin-3-yl)methyl)phenyl)-6,7-dihydro-5H-benzo[7]annulene-3-carboxylic acid, Isomer 1 (9),
8-(2,4-difluorophenyl)-9-(4-((1-(3,3-difluoropropyl)pyrrolidin-3-yl)methyl)phenyl)-6,7-dihydro-5H-benzo[7]annulene-3-carboxylic acid, Isomer 1 (10),
8-(4,4-dimethylcyclohex-1-en-1-yl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (11),
9-(4-((1-(3-fluoropropyl)pyrrolidin-3-yl)methyl)phenyl)-8-(2-methyl-3-(trifluoromethyl)phenyl)-6,7-dihydro-5H-benzo[7]annulene-3-carboxylic acid hydrochloride, Isomer 1 (12),
8-(3-chloro-2-(trifluoromethyl)phenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1(13),
9-(4-((1-(3-fluoropropyl)pyrrolidin-3-yl)methyl)phenyl)-8-(6-methoxypyridin-3-yl)-6,7-dihydro-5H-benzo[7]annulene-3-carboxylic acid, Isomer 1 (14),
8-(3-chloro-2-methylphenyl)-9-(4-((1-(3-fluoropropyl)pyrrolidin-3-yl)methyl)phenyl)-6,7-dihydro-5H-benzo[7]annulene-3-carboxylic acid, Isomer 1 (15),
8-(3-chloro-2-fluorophenyl)-9-(4-((1-(3-fluoropropyl)pyrrolidin-3-yl)methyl)phenyl)-6,7-dihydro-5H-benzo[7]annulene-3-carboxylic acid, Isomer 1 (16),
8-(4-fluoro-2-methylphenyl)-9-(4-((1-(3-fluoropropyl)pyrrolidin-3-yl)methyl)phenyl)-6,7-dihydro-5H-benzo[7]annulene-3-carboxylic acid, Isomer 1 (17),
8-(4-fluoro-2-(trifluoromethyl)phenyl)-9-(4-((1-(3-fluoropropyl)pyrrolidin-3-yl)methyl)phenyl)-6,7-dihydro-5H-benzo[7]annulene-3-carboxylic acid, Isomer 1 (18),
8-(2,4-difluorophenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (19),
8-(3-fluoro-2-methylphenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (20),
8-(2-chloro-3-fluorophenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (21),
8-(2,4-dichlorophenyl)-9-(4-((3-fluoro-1-(3-fluoropropyl)pyrrolidin-3-yl)methyl)phenyl)-6,7-dihydro-5H-benzo[7]annulene-3-carboxylic acid, 2,2,2-trifluoroacetic acid Isomer 2 (22),
8-(2,4-dichlorophenyl)-9-(4-((3-fluoro-1-(3-fluoropropyl)pyrrolidin-3-yl)methyl)phenyl)-6,7-dihydro-5H-benzo[7]annulene-3-carboxylic acid, 2,2,2-trifluoroacetic acid, Isomer 1 (23),
8-(2,4-dichlorophenyl)-9-(4-((1-(3-fluoropropyl)pyrrolidin-3-yl)methyl)phenyl)-6,7-dihydro-5H-benzo[7]annulene-3-carboxylic acid, (R) Isomer (24),
3-(2,4-dichlorophenyl)-4-(4-((1-(3-fluoropropyl)pyrrolidin-3-yl)methyl)phenyl)-2H-thiochromene-7-carboxylic acid hydrochloride, Isomer 1 (25),
8-(2,4-dichlorophenyl)-9-(4-(difluoro(1-(3-fluoropropyl)pyrrolidin-3-yl)methyl)phenyl)-6,7-dihydro-5H-benzo[7]annulene-3-carboxylic acid, Isomer 1 (26),
8-(2-chloro-4-fluorophenyl)-9-(4-((1-(3,3-difluoropropyl)pyrrolidin-3-yl)methyl)phenyl)-6,7-dihydro-5H-benzo[7]annulene-3-carboxylic acid hydrochloride, Isomer 1 (27),
9-(4-((1-(3,3-difluoropropyl)pyrrolidin-3-yl)methyl)phenyl)-8-(2-methyl-3-(trifluoromethyl)phenyl)-6,7-dihydro-5H-benzo[7]annulene-3-carboxylic acid hydrochloride, Isomer 1 (28),
8-(2,4-dichlorophenyl)-9-(4-((1-(3-fluoropropyl)pyrrolidin-3-yl)methyl)phenyl)-6,7-dihydro-5H-benzo[7]annulene-3-ol hydrochloride, Isomer 1 (29),
9-(4-((1-(3-fluoropropyl)pyrrolidin-3-yl)methyl)phenyl)-8-(3-methyl-2-(trifluoromethyl)phenyl)-6,7-dihydro-5H-benzo[7]annulene-3-carboxylic acid, Isomer 1 (30),
8-(2,4-dichlorophenyl)-9-(4-((1-(3,3-difluoropropyl)pyrrolidin-3-yl)methyl)phenyl)-6,7-dihydro-5H-benzo[7]annulen-3-ol hydrochloride, Isomer 1 (31),
9-(4-((1-(3,3-difluoropropyl)pyrrolidin-3-yl)methyl)phenyl)-8-(4-fluoro-2,6-dimethylphenyl)-6,7-dihydro-5H-benzo[7]annulene-3-carboxylic acid hydrochloride, Isomer 1 (32),
9-(4-((1-(3,3-difluoropropyl)pyrrolidin-3-yl)methyl)phenyl)-8-(2-(trifluoromethyl)phenyl)-6,7-dihydro-5H-benzo[7]annulene-3-carboxylic acid (33),
9-(4-((1-(3,3-difluoropropyl)pyrrolidin-3-yl)methyl)phenyl)-8-(3-methyl-2-(trifluoromethyl)phenyl)-6,7-dihydro-5H-benzo[7]annulene-3-carboxylic acid hydrochloride, Isomer 1 (34),
8-(4-fluoro-2,6-dimethylphenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (35),
6-(2,4-dichlorophenyl)-5-(4-((1-(3-fluoropropyl)pyrrolidin-3-yl)methyl)phenyl)-7,8-dihydronaphthalene-2-carboxylic acid hydrochloride, Isomer 1 (36),
8-(4-chlorophenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (37),
9-(4-((1-(3-fluoropropyl)pyrrolidin-3-yl)methyl)phenyl)-8-(4-(trifluoromethyl)phenyl)-6,7-dihydro-5H-benzo[7]annulene-3-carboxylic acid hydrochloride, Isomer 1 (38),
9-(4-((1-(3-fluoropropyl)pyrrolidin-3-yl)methyl)phenyl)-8-(3-(trifluoromethyl)phenyl)-6,7-dihydro-5H-benzo[7]annulene-3-carboxylic acid hydrochloride, Isomer 1 (39),
8-(3-chlorophenyl)-9-(4-((1-(3-fluoropropyl)pyrrolidin-3-yl)methyl)phenyl)-6,7-dihydro-5H-benzo[7]annulene-3-carboxylic acid hydrochloride, Isomer 1 (40),
9-(4-((1-(3-fluoropropyl)pyrrolidin-3-yl)methyl)phenyl)-7-methyl-8-(4-(trifluoromethyl)phenyl)-6,7-dihydro-5H-benzo[7]annulene-3-carboxylic acid, Isomer 1 (41),
9-(4-((1-(3-fluoropropyl)pyrrolidin-3-yl)methyl)phenyl)-7-methyl-8-(4-(trifluoromethyl)phenyl)-6,7-dihydro-5H-benzo[7]annulene-3-carboxylic acid, Isomer 2 (42),
4-(2,4-dichlorophenyl)-5-(4-((1-(3-fluoropropyl)pyrrolidin-3-yl)methyl)phenyl)-2,3-dihydrobenzo[b]thiepin-8-ol, Isomer 1 (43),
4-(2-chlorophenyl)-5-(4-((1-(3-fluoropropyl)pyrrolidin-3-yl)methyl)phenyl)-2,3-dihydrobenzo[b]thiepin-8-ol, Isomer 1 (44).

20. A process for preparing a compound of formula (I) as defined in claim 1, or a pharmaceutically acceptable salt thereof, wherein a compound of formula 1G 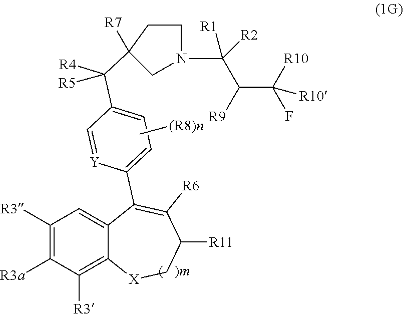
wherein R1, R2, R3′, R3″, R4, R5, R6, R7, R8, R9, R10, R10′, R11, n, m, X and Y are as defined in claim 1 and R3a is a hydrogen atom, a carboxylic ester, or protected OH, is converted to compound of formula (I), in presence of a source of hydroxide ions, said step being optionally preceded by a step for obtaining compound 1G, wherein a compound of formula 1F 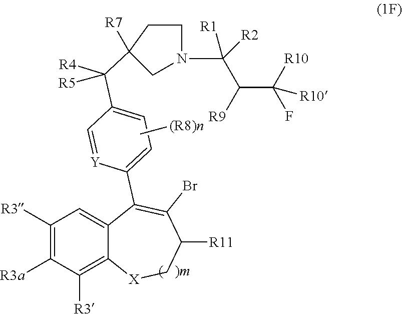
wherein R1, R2, R3′, R3″, R4, R5, R7, R8, R9, R10, R10′, R11, n, m, X and Y are as defined in claim 1 and R3a is as defined above,
is subjected to a Suzuki coupling with a boronic reagent R6-B(OR′)₂, wherein —B(OR′)₂ is a boronic acid or a pinacolate ester and R6 is as defined in claim 1.

21. A process for preparing a compound of formula (I) as defined in claim 1, or a pharmaceutically acceptable salt thereof, wherein a compound of formula 1Fa 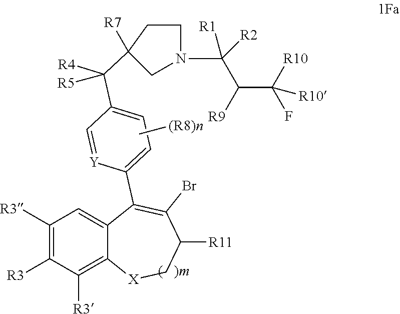
wherein R1, R2, R3, R3′, R3″, R4, R5, R7, R8, R9, R10, R10′, R11, n, m, X and Y are as defined in claim 1, is submitted to a Suzuki coupling with a boronic reagent R6-B(OR′)₂, wherein —B(OR′)₂ is a boronic acid or a pinacolate ester and R6 is defined as in claim 1, said step being optionally preceded by a step for obtaining compound 1Fa, wherein a compound of formula 1F 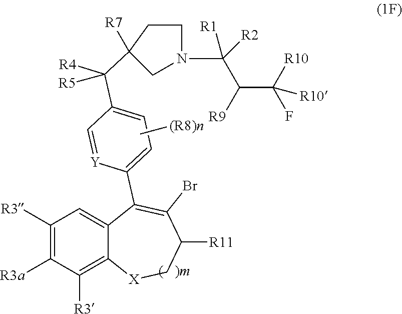
wherein R1, R2, R3′, R3″, R4, R5, R7, R8, R9, R10, R10′, R11, n, m, X and Y are as defined in claim 1 and R3a is a hydrogen atom, a carboxylic ester, or protected OH,
is converted to a compound 1Fa in the presence of a source of hydroxide ions.

22. A compound selected from compounds of formula 1E, 1F, 1G and 1Fa, or a pharmaceutically acceptable salt thereof: 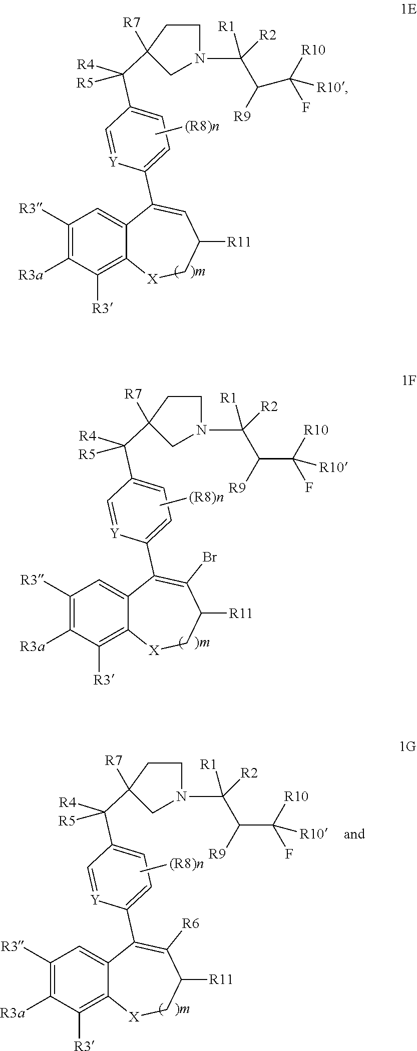 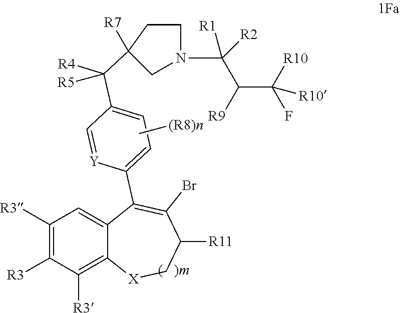
wherein
R1 and R2 independently represent a hydrogen atom or a deuterium atom;
R3 represents a hydrogen atom, a —COOH group or a —OH group;
R3′ and R3″ independently represent a hydrogen atom, a methyl group, a methoxy group, a chlorine atom, a fluorine atom, or a cyano group;
R4 and R5 independently represent a hydrogen atom, a fluorine atom, a —NH₂ group, a (C₁-C₃)alkyl group, a (C₁-C₃)alkoxy group, or a —OH group; or R4 and R5 together form an oxo group or R4 and R5 together form a ═NOCH₃ group or a (C₃-C₅)cycloalkyl group with the carbon atom to which they are attached;
R7 represents a hydrogen atom, a methyl group, a —OH group or a fluorine atom;
or alternatively R4 and R7 together form a cyclopropyl group together with the bond to which they are attached, that gives with the adjacent pyrrolidine group an azaspiro[2.4]heptane;
R6 represents a group selected from:
a phenyl group, said phenyl group being optionally substituted by 1 to 3 substituents independently selected from a halogen atom; a (C₁-C₆)alkyl group optionally substituted with a cyano group or a —OH group; a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group;
a fused phenyl group, selected from phenyl groups fused with a (C₃-C₆)cycloalkyl, which (C₃-C₆)cycloalkyl ring optionally comprises an unsaturation and wherein the fused phenyl is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
a bicyclic group comprising 5 to 12 carbon atoms, optionally comprising 1 to 2 unsaturations; optionally substituted with 1 to 4 substituents independently selected from: a fluorine atom, a —OH group, a (C₁-C₃)alkyl group, a (C₁-C₃)fluoroalkyl group, a (C₁-C₃)alkoxy group, a (C₁-C₃)fluoroalkoxy group and an oxo group;
a heteroaryl group comprising 2 to 9 carbon atoms and comprising from 1 to 3 heteroatoms independently selected from oxygen, nitrogen and sulfur, and at least 5 atoms including carbon atoms and heteroatoms, said heteroaryl group being optionally substituted with 1 to 3 substituents independently selected from a halogen atom, a (C₁-C₆)alkyl group, a (C₁-C₆)fluoroalkyl group, a (C₁-C₆)alkoxy group, a (C₁-C₆)fluoroalkoxy group, a cyano group, a carbamoyl group and a —OH group;
a cycloalkyl group comprising 3 to 7 carbon atoms, said cycloalkyl group being saturated or partially saturated and being optionally substituted with 1 to 4 substituents independently selected from:
a fluorine atom, a —OH group, a (C₁-C₃)alkyl group, a (C₁-C₃)fluoroalkyl group, a (C₁-C₃)alkoxy group, a (C₁-C₃)fluoroalkoxy group, an oxo group,
a (C₃-C₆)cycloalkyl group, and a phenyl group, said (C₃-C₆)cycloalkyl or phenyl groups being optionally substituted with one or two halogen atom(s) or (C₁-C₃)alkyl group(s);
a (C₃-C₆)cycloalkyl(C₁-C₃)alkyl group, optionally substituted on the cycloalkyl with 1 to 4 substituents independently selected from: a fluorine atom, a —OH group, a (C₁-C₄)alkyl group, a (C₁-C₃)fluoroalkyl group, a (C₁-C₃)fluoroalkoxy group and an oxo group;
a 4 to 7 membered-heterocycloalkyl group comprising 1 or 2 heteroatoms independently selected from oxygen, nitrogen and sulfur, said heterocycloalkyl group being saturated or partially saturated and being optionally substituted with 1 to 3 substituents independently selected from: a fluorine atom, a (C₁-C₃)alkyl group, a (C₁-C₃)fluoroalkyl group, a (C₁-C₃)fluoroalkoxy group, an oxo group, a (C₁-C₃)alkoxy group and a —OH group;
a (C₁-C₆)alkyl group, said alkyl group being optionally substituted with 1 to 4 substituents independently selected from: a fluorine atom, a (C₁-C₃)alkoxy group, a (C₁-C₃)fluoroalkoxy group and a —OH group; and
a phenyl(C₁-C₂)alkyl group, said phenyl group being optionally substituted with 1 to 3 substituents independently selected from a halogen atom; a (C₁-C₃)alkyl group; a (C₁-C₃)fluoroalkyl group; a (C₁-C₃)alkoxy group; a (C₁-C₃)fluoroalkoxy group; a cyano group; and a —OH group;
X represents —CH₂—, —O—, or —S—;
Y represents —CH═, —N═ or —CR″═, wherein R″ represents a (C₁-C₃)alkyl group or a halogen atom, a cyano group, or a (C₁-C₃)fluoroalkyl group;
R8 independently represents a (C₁-C₃)alkyl group, a halogen atom, a cyano group, or a (C₁-C₃)fluoroalkyl group;
R9 represents a hydrogen atom or a fluorine atom;
R10 and R10′ independently represent a hydrogen atom or a fluorine atom;
R11 represents a hydrogen atom, or a (C₁-C₃)alkyl group or a cyclopropyl;
n is 0, 1 or 2,
m is 0 or 1,
and R3a is a hydrogen atom, a carboxylic ester, or protected OH.

23. A compound of formula 1D or 1D′, or a pharmaceutically acceptable salt thereof: 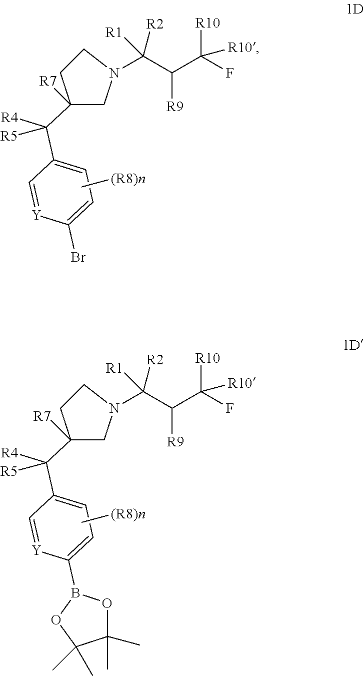
wherein
R1 and R2 independently represent a hydrogen atom or a deuterium atom;
R4 and R5 independently represent a hydrogen atom, a fluorine atom, a —NH₂ group, a (C₁-C₃)alkyl group, a (C₁-C₃)alkoxy group, or a —OH group; or R4 and R5 together form an oxo group or R4 and R5 together form a ═NOCH₃ group or a (C₃-C₅)cycloalkyl group with the carbon atom to which they are attached;
R7 represents a hydrogen atom, a methyl group, a —OH group or a fluorine atom;
or alternatively R4 and R7 together form a cyclopropyl group together with the bond to which they are attached, that gives with the adjacent pyrrolidine group an azaspiro[2.4]heptane;
Y represents —CH═, —N═ or —CR″═, wherein R″ represents a (C₁-C₃)alkyl group or a halogen atom, a cyano group, or a (C₁-C₃)fluoroalkyl group;
R8 independently represents a (C₁-C₃)alkyl group, a halogen atom, a cyano group, or a (C₁-C₃)fluoroalkyl group;
R9 represents a hydrogen atom or a fluorine atom;
R10 and R10′ independently represent a hydrogen atom or a fluorine atom; and
n is 0, 1 or 2.

24. (canceled)

25. A pharmaceutical composition, characterized in that it comprises a compound of formula (I) according to claim 1, or a pharmaceutically acceptable salt thereof, and at least one pharmaceutically acceptable excipient.

26. A method for inhibiting and degrading estrogen receptors, which comprises administering to a subject in need thereof a therapeutically effective amount of a compound of claim 1.

27. A method of treating ovulatory dysfunction, cancer, endometriosis, osteoporosis, benign prostatic hypertrophy or inflammation, which comprises administering to a subject in need thereof a therapeutically effective amount of a compound of claim 1.

28. The method according to claim 27, which comprises the treatment of cancer.
